(19) United States
(12) Patent Application Publication
(10) Pub. No.: US 2004/0091908 A1
(43) Pub. Date: May 13, 2004

(54) Crystal structures of Kv channel proteins and uses thereof

(76) Inventors: Weixin Xu, Acton, MA (US); Zhangbao Xu, Tewksbury, MA (US); Laura Long Lin, Weston, MA (US); Stephane Hubert Olland, Arlington, MA (US); Lidia Mosyak, Newton, MA (US); William Stuart Somers, Lexington, MA (US)

Correspondence Address: Craig J. Arnold, Amster, Rothstein & Ebenstein, 90 Park Avenue, New York, NY, 10016, US

(21) Appl. No.: 10/611,718

(22) Filed: Jul. 1, 2003

Related U.S. Application Data
(60) Provisional application No. 60/394,370, filed on Jul. 8, 2002.

(51) Int. Cl. C12Q 1/68; G01N 33/53; G06F 19/00; G01N 33/48; G01N 33/50; C07K 14/705

(52) U.S. Cl. 435/6; 435/7.1; 530/350; 702/19

(57) ABSTRACT

This invention is directed to the crystal structures of Kv channel proteins, and to the use of these structures in rational drug design methods to identify agents that may interact with active sites of Kv channel proteins. Such agents may be useful as therapeutic agents.

### CROSS REFERENCE TO RELATED APPLICATIONS

This application claims the benefit of U.S. Provisional Application No. 60/394,370, filed Jul. 8, 2002.

### FIELD OF THE INVENTION

The present invention relates to the identification of the crystal structures of Kv Channel Proteins, KCHIP and Kv4.3 T1, and the use of the structures for designing new drugs.

### BACKGROUND OF THE INVENTION

Mammalian cell membranes are important to the structural integrity and activity of many cells and tissues. Of particular interest in membrane physiology is the study of transmembrane ion channels which act to directly control a variety of pharmacological, physiological, and cellular processes. Numerous ion channels have been identified including calcium, sodium, and potassium channels, each of which has been investigated to determine their roles in vertebrate and insect cells.

Because of their involvement in maintaining normal cellular homeostasis, much attention has been given to potassium channels. A number of these potassium channels open in response to change in the cell membrane potential. Many voltage-gated potassium channels have been identified and characterized by their electrophysiological and pharmacological properties. Potassium currents are more diverse than sodium or calcium currents and are further involved in determining the response of a cell to external stimuli.

The diversity of potassium channels and their important physiological role highlights their potential as targets for developing therapeutic agents for various diseases. One of the best characterized classes of potassium channels are the voltage-gated potassium channels. The prototypical member of this class is the protein encoded by the Shaker gene in Drosophila melanogaster. Proteins of the Shal or Kv4 family are a type of voltage-gated potassium channel that underlies many of the native inactivating currents (A-type currents in neurons and Iₜₒ in cardiac cells (Dixon et al. 1996) that have been recorded from different primary cells. In neurons, Kv4 channels and the A-type currents may play an important role in modulation of firing rate, action potential initiation and in controlling dendritic responses to synaptic inputs. Kv4 channels also have a major role in the repolarization of cardiac action potentials.

The fundamental function of a neuron is to receive, conduct, and transmit signals. Despite the varied purposes of the signals carried by different classes of neurons, the general form of the signal is similar, consisting of changes in the electrical potential across the plasma membrane of the neuron. The plasma membrane of a neuron contains voltage-gated cation channels, which are responsible for propagating this electrical potential (also referred to as an action potential or nerve impulse) across and along the plasma membrane. Signaling in cardiac muscle cells is similar, although the ultimate output of the cell differs, comprising cellular contraction rather than neurotransmitter release.

The Kv family of channels includes, among others: (1) the delayed-rectifier potassium channels, which repolarize the membrane after each action potential to prepare the cell to fire again; and (2) the rapid inactivating (A-type) potassium channels, which are active predominantly at subthreshold voltages and act to control the rate at which excitable cells reach firing threshold. In addition to being critical for action potential conduction, Kv channels also control the response to depolarizing, e.g., synaptic, inputs and play a role in neurotransmitter release. As a result of these activities, voltage-gated potassium channels are key regulators of neuronal and cardiac excitability (Hille, 1992). Also see refs (Sheng et al., 1992, Serodio & Rudy, 1998, Serodio et al., 1996).

There is a tremendous structural and functional diversity within the Kv potassium channel superfamily. This diversity is generated both by the existence of multiple genes and by alternative splicing of RNA transcripts produced from the same gene. Nonetheless, the amino acid sequences of the known Kv potassium channels show high similarity. All appear to be comprised of four, pore forming α-subunits and some are known to have four tightly associated cytoplasmic (β-subunit) polypeptides (Jan et al., 1990, Pongs et al., 1999, Gulbis et al., 2000). The known Kv channels fall into multiple sub-families, of which Kv4.2 and Kv4.3 are examples of Kv channels α-subunits) related to the Shal channels in D. melanogaster.

Kv4 channels are highly expressed in brain and in cardiac tissues. In brain, Kv4.2 and Kv4.3 are expressed in the somatodendritic membranes of these cells, where they are thought to contribute to the rapidly inactivating (A-type) K⁺ conductance (Sheng et al., 1992). These somatodendritic A-type Kv channels may contribute to integration of sub-threshold synaptic responses and the conductance of back-propagating action potentials (Hoffman et al., 1997). Kv4 channels likely are involved in various processes in the brain, including learning and memory and the release of various neurotransmitters. Although Kv4 channels give rise to A-type currents in heterologous cells, these currents differ significantly from native A-type currents. Four Kv Channel Interacting Proteins (KCHIPs) that bind to the cytoplasmic amino termini of Kv4 channels have previously been identified (An et al 2000). There are experimental evidences showing that potential binding site for KCHIPs is located in the tetramerization domain (T1) of Kv4 (Bähring et al., 2001). The T1 domain has been shown to supervise the proper assembly of specific tetrameric channels (Li et al., 1992, Shen et al., 1993).

Expression of KCHIP and Kv4 together dramatically enhances the channel activity by modulating the density, inactivation kinetics, and rate of recovery from inactivation of Kv4 currents in heterologous cells. These KCHIPs have four Ca²⁺-binding EF-hand-like domains which bind calcium ions, and are novel members of the recoverin family of calcium binding proteins. All KCHIPs co-localize and co-immunoprecipitate with Kv4 α-subunits in rat brain, and are thus integral components of native Kv4 channel complexes. As the activity and density of neuronal A-type currents tightly control responses to excitatory synaptic inputs, these KCHIPs may regulate A-type currents, and hence neuronal excitability, in response to changes in intracellular calcium.

Thus, KCHIP proteins which interact with and modulate the activity of Kv4 potassium channel proteins provide novel molecular drug targets to modulate cellular excitability, e.g., action potential conduction, somatodendritic excitability, neurotransmitter release, and cellular contraction, in cells expressing these channels. In addition, detection of genetic lesions in the genes encoding these proteins could be used to diagnose central nervous system and cardiac disorders such as epilepsy, anxiety, depression, age-related memory loss, migraine, obesity, Parkinsons disease, Alzheimer's disease and arrhythmias.

### SUMMARY OF THE INVENTION

The present invention provides a crystallized Kv channel-interacting protein 1 (KCHIP-1) having one molecule of KCHIP-1 in the asymmetric unit.

The present invention also provides a crystallized potassium channel Kv4.3 T1 domain (Kv4.3 T1) having two monomers of Kv4.3 T1 in the asymmetric unit.

Additionally, the present invention provides a three dimensional model of KCHIP-1 as derived by x-ray diffraction data of the KCHIP-1 crystal. Specifically, the three dimensional model of KCHIP-1 is defined by the relative structural coordinates for KCHIP-1 according to FIG. 4, ± a root mean square deviation from the backbone atoms of said amino acids of not more than 1.5 Å. The three dimensional model of KCHIP-1 is useful for a number of applications, including, but not limited to, the visualization, identification and characterization of various active sites of KCHIP-1. The active site structures may then be used to design various agents which interact with KCHIP-1.

The present invention also provides a three dimensional model of Kv4.3 T1 as derived by x-ray diffraction data of the Kv4.3 T1 crystal. Specifically, the three dimensional model of Kv4.3 T1 is defined by the relative structural coordinates for Kv4.3 T1 according to FIG. 5, ± a root mean square deviation from the backbone atoms of said amino acids of not more than 1.5 Å. The three dimensional model of Kv4.3 T1 is useful for a number of applications, including, but not limited to, the visualization, identification and characterization of various active sites of Kv4.3 T1. The active site structures may then be used to design various agents which interact with Kv4.3 T1.

In addition, the present invention provides a method for identifying an agent that interacts with KCHIP-1, comprising the steps of: (a) generating a three dimensional model of KCHIP-1 using the relative structural coordinates of KCHIP-1 according to FIG. 4, ± a root mean square deviation from the backbone atoms of said amino acids of not more than 1.5 Å; and (b) employing said three-dimensional model to design or select an agent that interacts with KCHIP-1.

Still further, the present invention provides a method for identifying an agent that interacts with Kv4.3 T1, comprising the steps of: (a) generating a three dimensional model of Kv4.3 T1 using the relative structural coordinates of Kv4.3 T1 according to FIG. 5, ± a root mean square deviation from the backbone atoms of said amino acids of not more than 1.5 Å; and (b) employing said three-dimensional model to design or select an agent that interacts with Kv4.3 T1.

Finally, the present invention provides agents identified using the foregoing methods. Small molecules or other agents which inhibit or otherwise interfere with binding to the KCHIP-1 or Kv4.3 T1 proteins may be useful as therapeutic agents.

Additional objects of the present invention will be apparent from the description which follows.

### BRIEF DESCRIPTION OF THE FIGURES

FIG. 1 is a ribbon representation of KCHIP-1.

FIG. 2 is a ribbon representation of Kv4.3 T1 dimer. Layers 1, 2, 3 and 4 are labeled. There is one Zn²⁺ per monomer. On Zn coordination is shown in the dimer interface.

FIG. 3 is a representation of the tetrameric interface of Shal T1 domain. Only polar interactions are shown.

FIG. 4 provides the atomic structural coordinates for KCHIP-1 as derived by X-ray diffraction of a KCHIP-1 crystal. “Atom type” refers to the atom whose coordinates are being measured. “Residue” refers to the type of residue of which each measured atom is a part—i.e., amino acid, cofactor, ligand or solvent. The “x, y and z” coordinates indicate the Cartesian coordinates of each measured atom's location in the unit cell (Å). “Occ” indicates the occupancy factor. “B” indicates the “B-value”, which is a measure of how mobile the atom is in the atomic structure (Å²).

FIG. 5 provides the atomic structural coordinates for Kv4.3 T1 as derived by X-ray diffraction of a Kv4.3 T1 crystal. “Atom type” refers to the atom whose coordinates are being measured. “Residue” refers to the type of residue of which each measured atom is a part—i.e., amino acid, cofactor, ligand or solvent. The “x, y and z” coordinates indicate the Cartesian coordinates of each measured atom's location in the unit cell (Å). “Occ” indicates the occupancy factor. “B” indicates the “B-value”, which is a measure of how mobile the atom is in the atomic structure (Å²).

FIG. 6 is the amino acid sequence of KCHIP-1 that was expressed in E. coli and used for crystallization. The sequence shown includes KCHIP-1, with two additional amino acids (i.e., Val and Glu) and six His-tag at the C-terminal.

FIG. 7 is the amino acid sequence of the T1 domain of Kv4.3 that was expressed in E. coli and used for crystallization. The sequence shown includes Kv4.3 T1, with one additional amino acid (i.e., Met) at the N-terminal, and two additional amino acids (i.e., Leu and Glu) and six His-tag at the C-terminal.

### DETAILED DESCRIPTION OF THE INVENTION

As used herein, the following terms and phrases shall have the meanings set forth below:

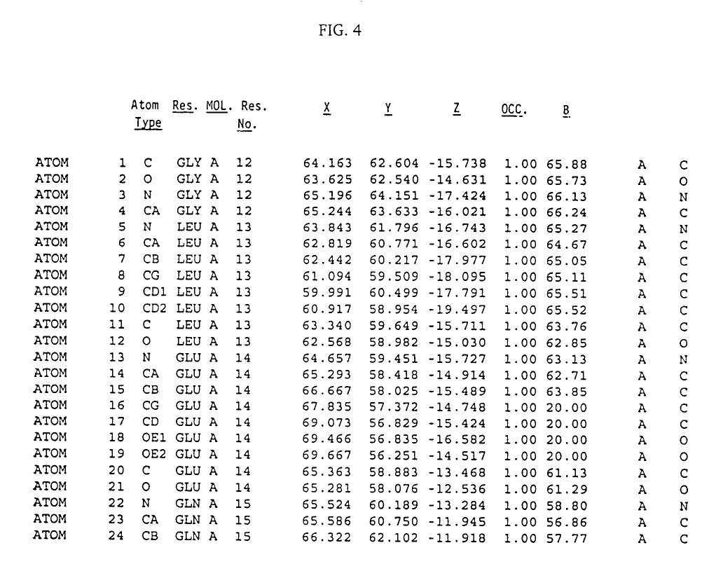

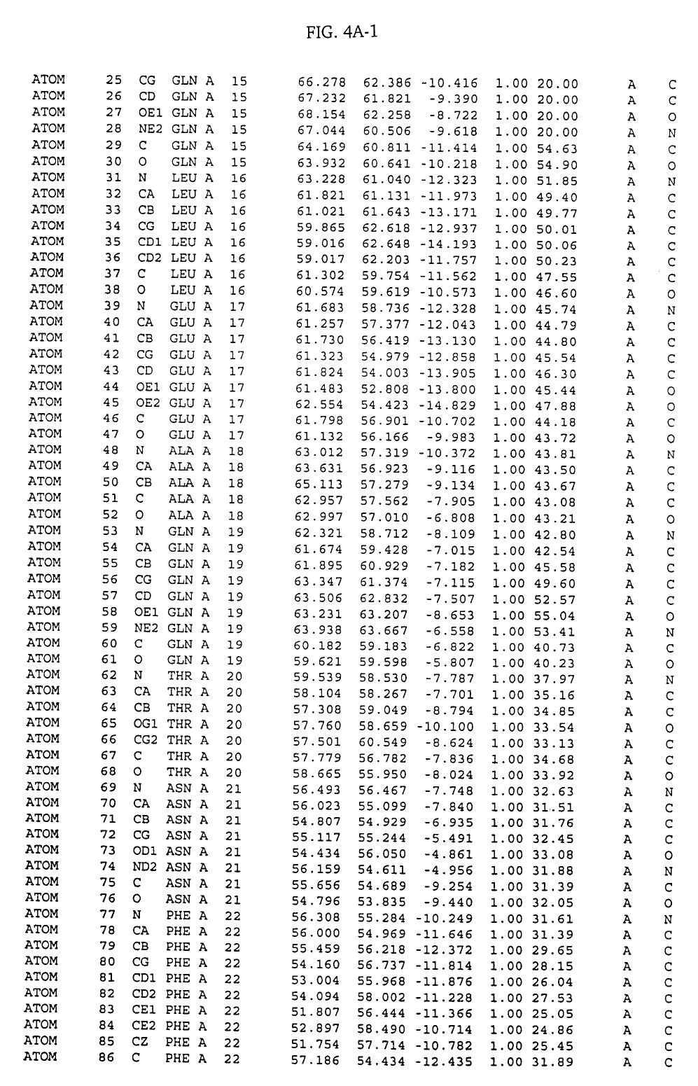

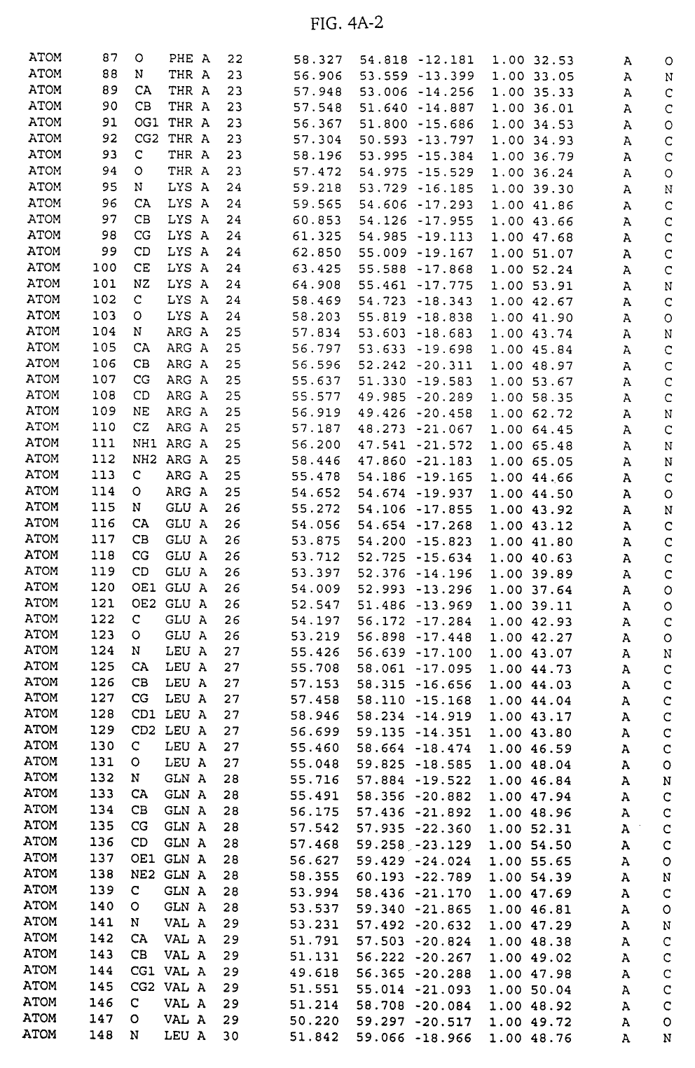

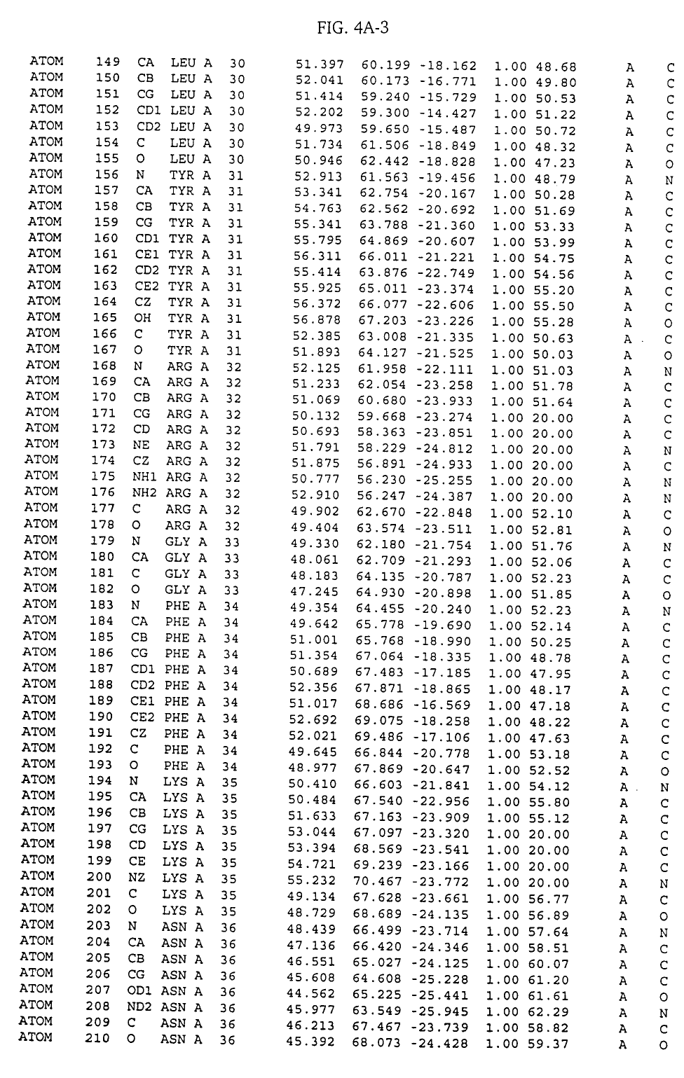

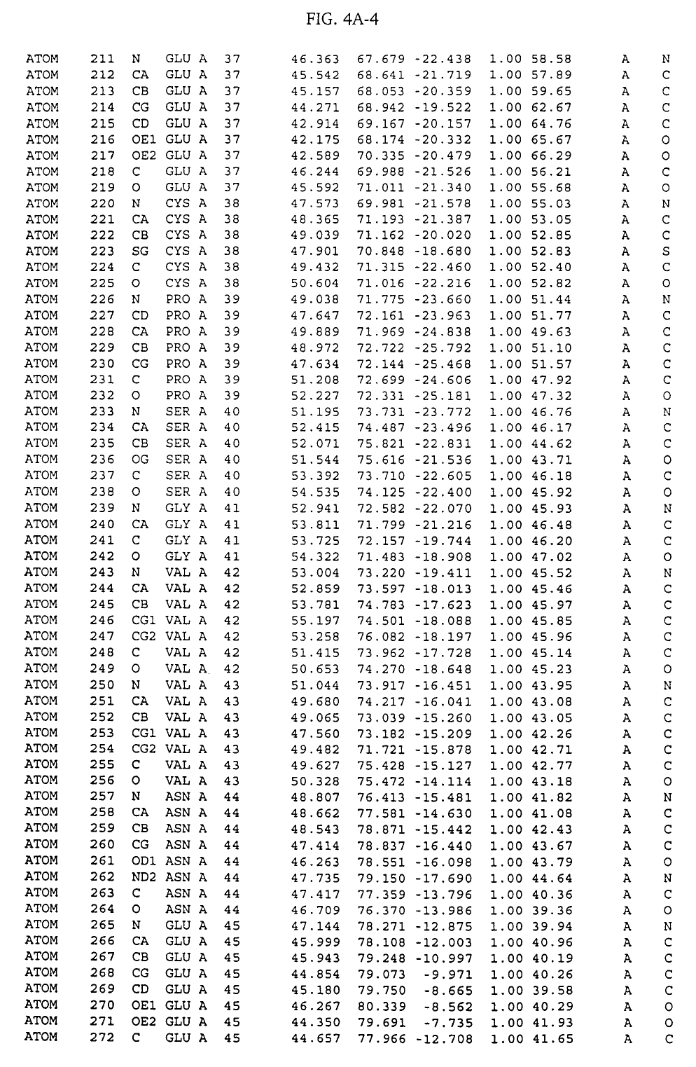

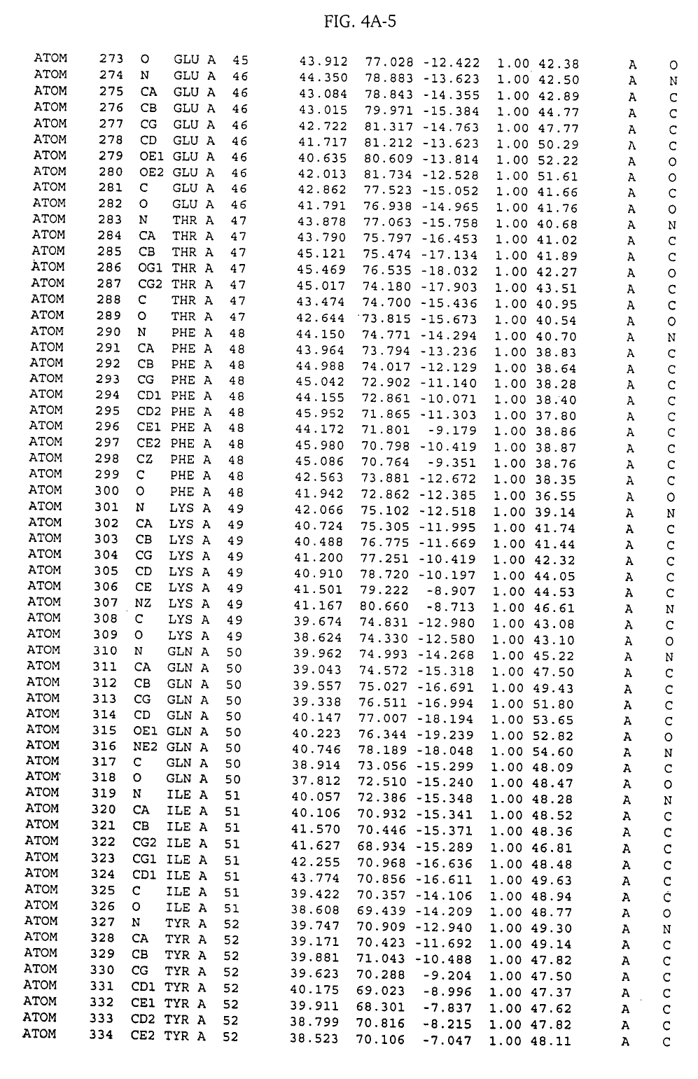

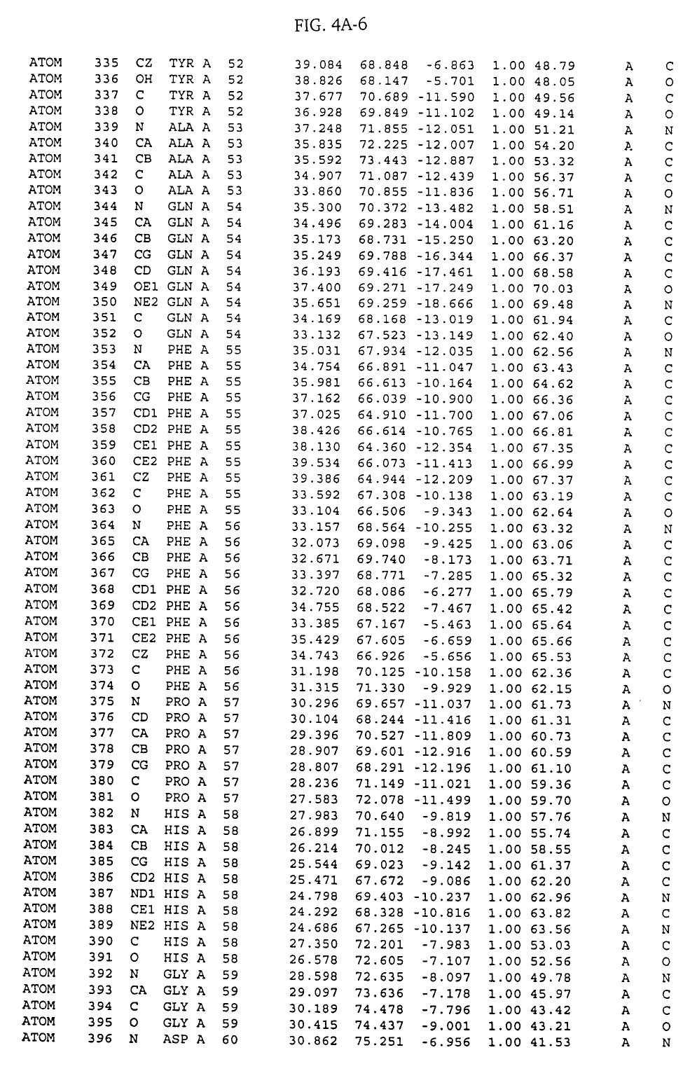

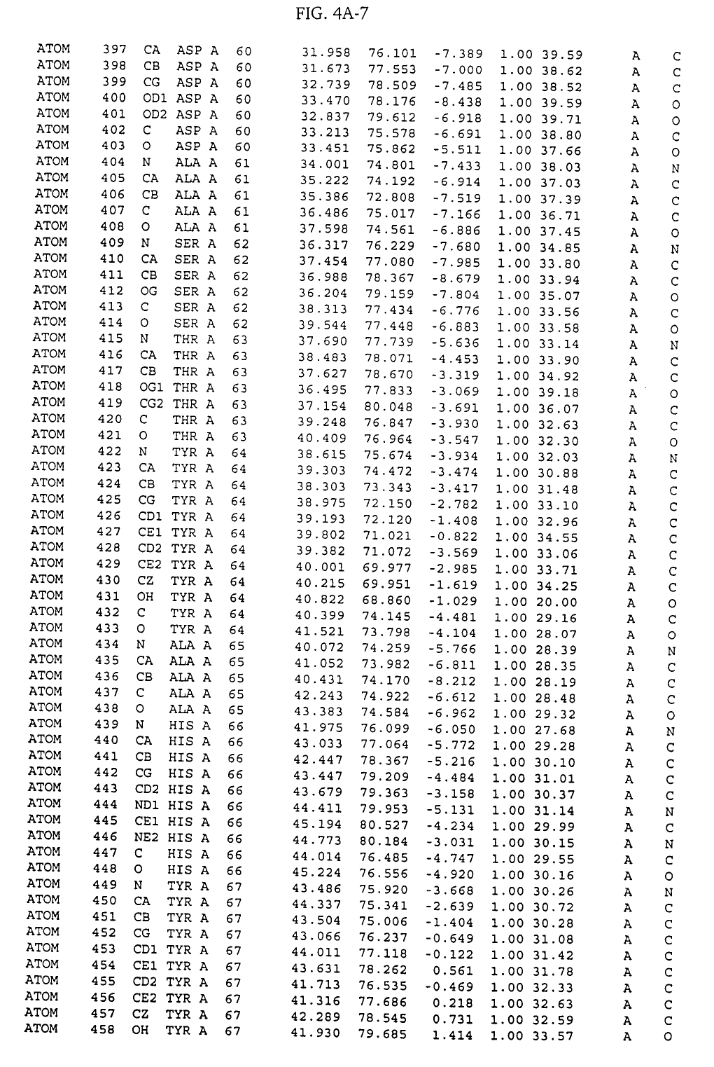

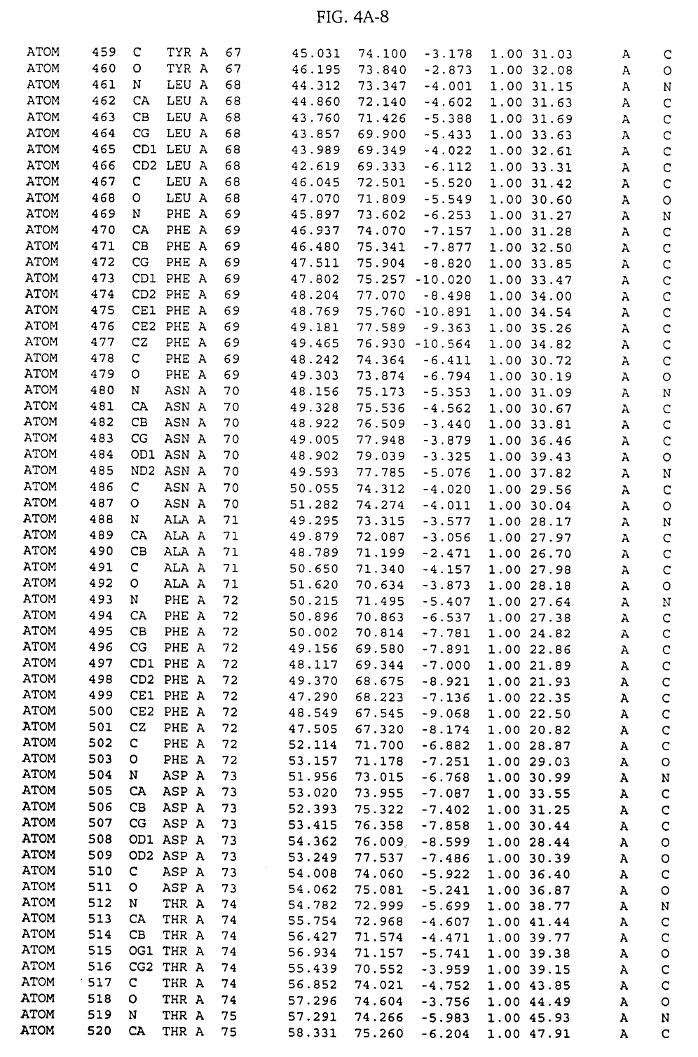

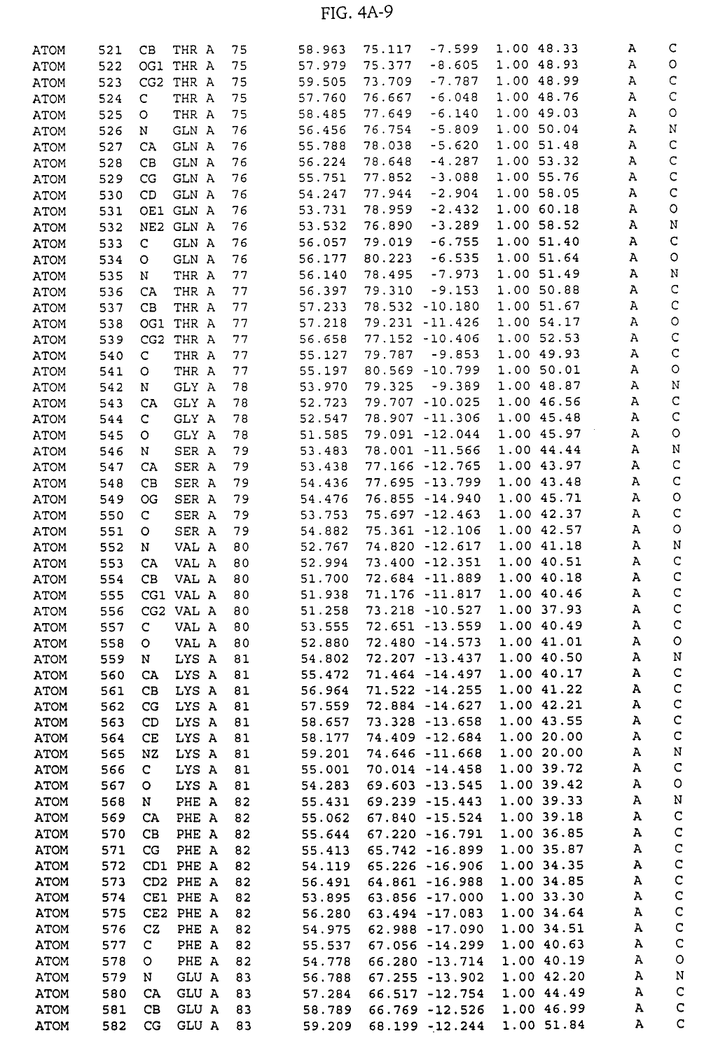

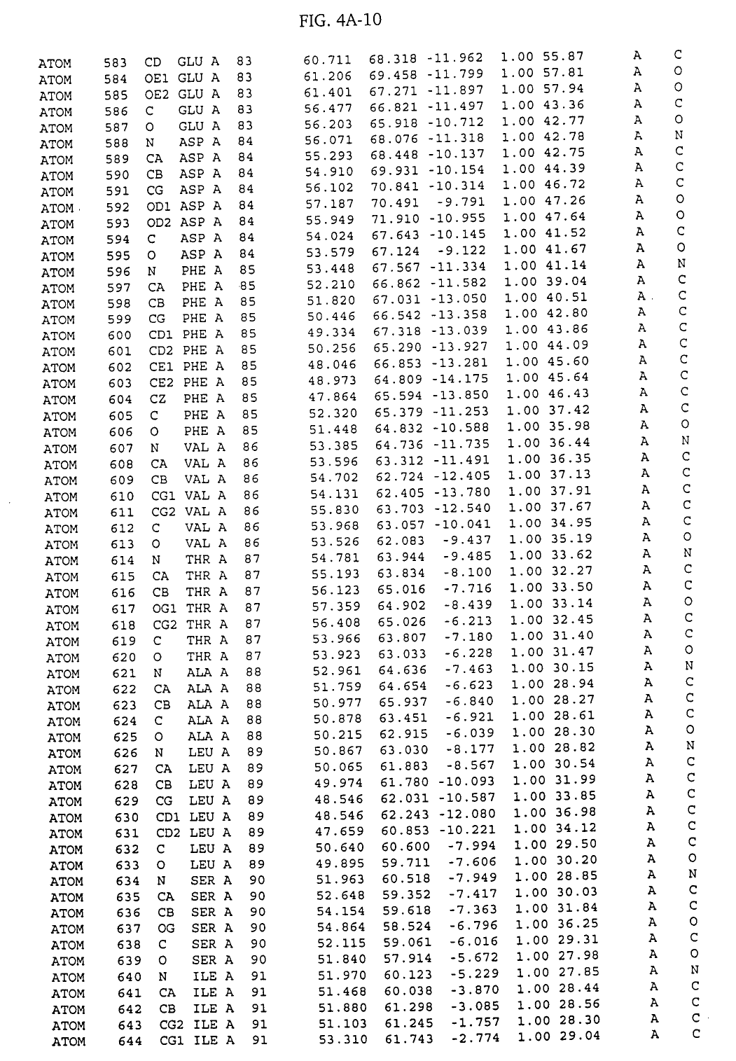

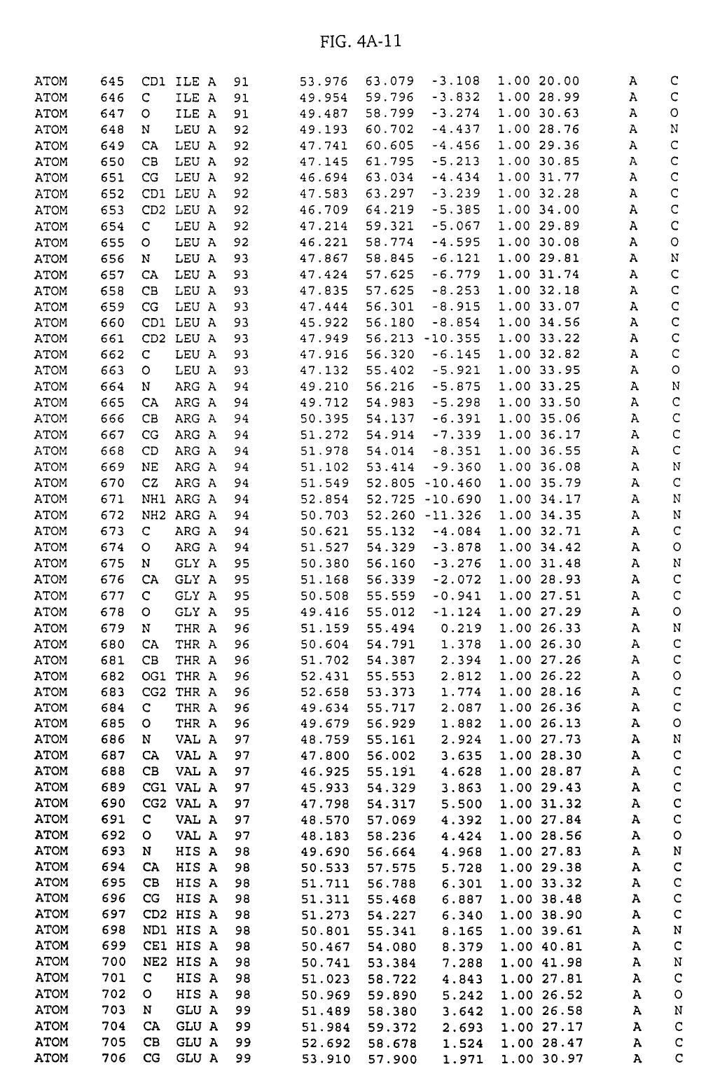

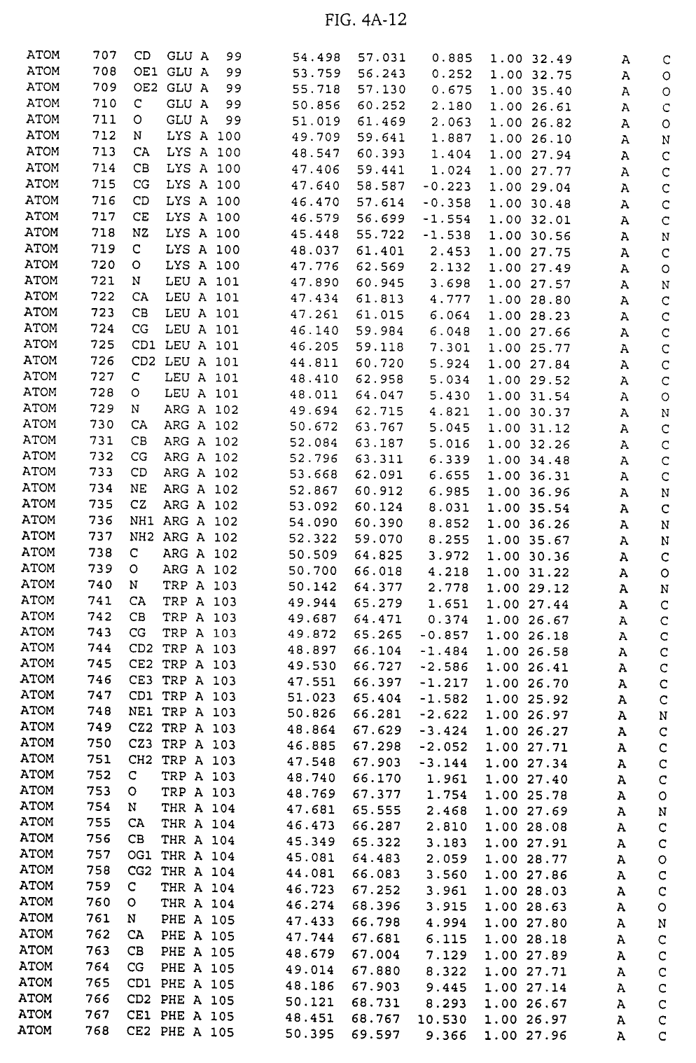

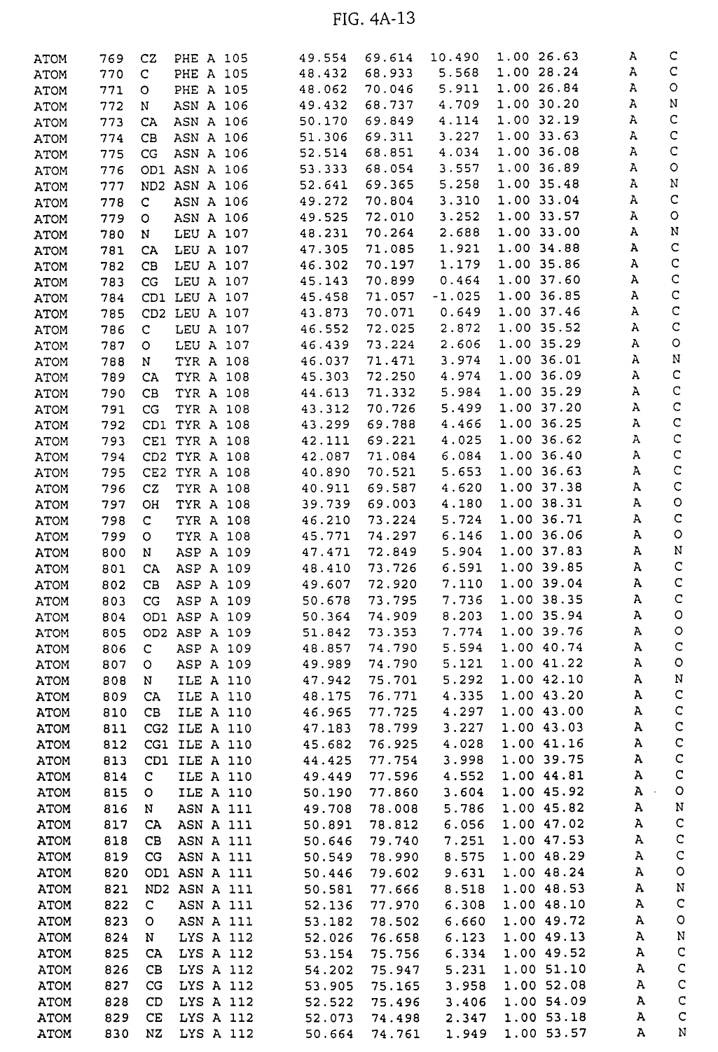

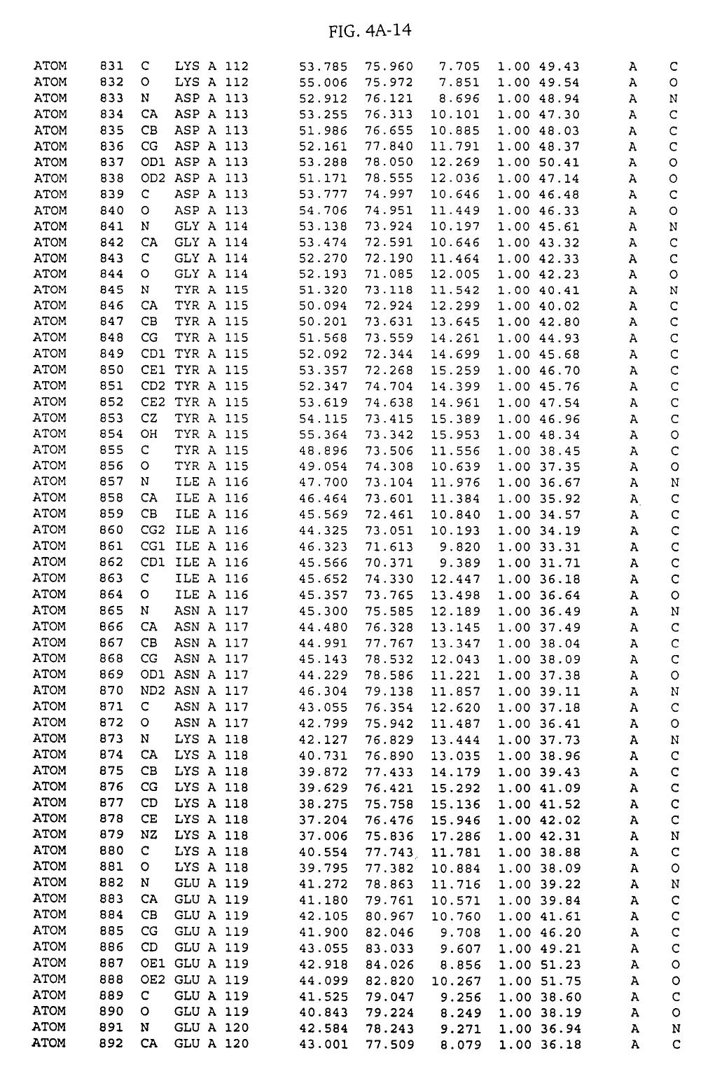

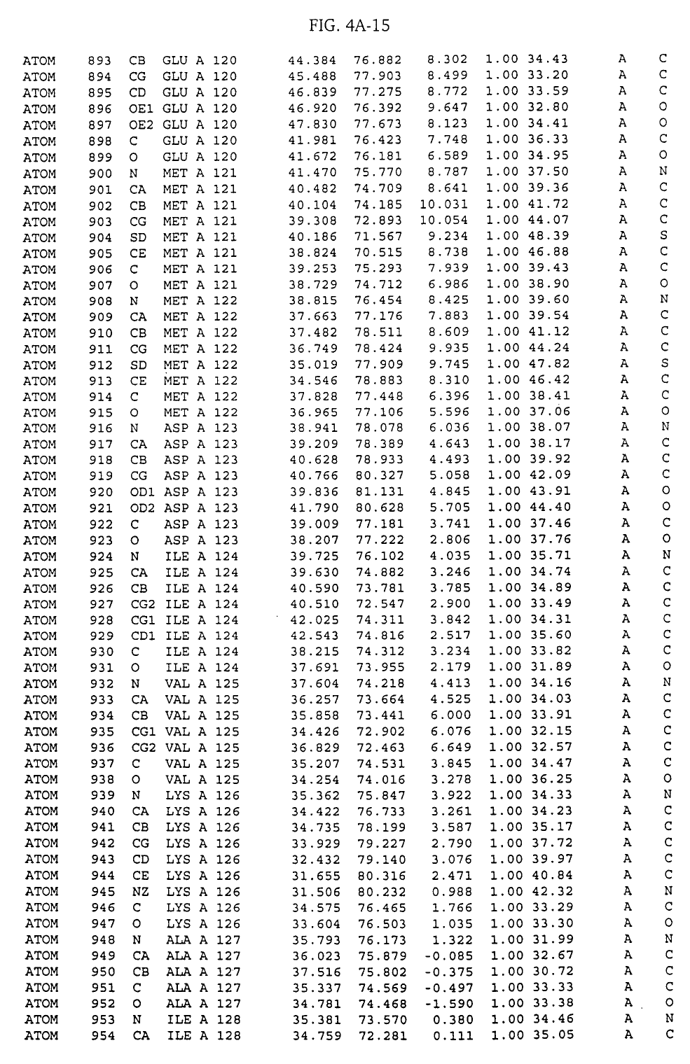

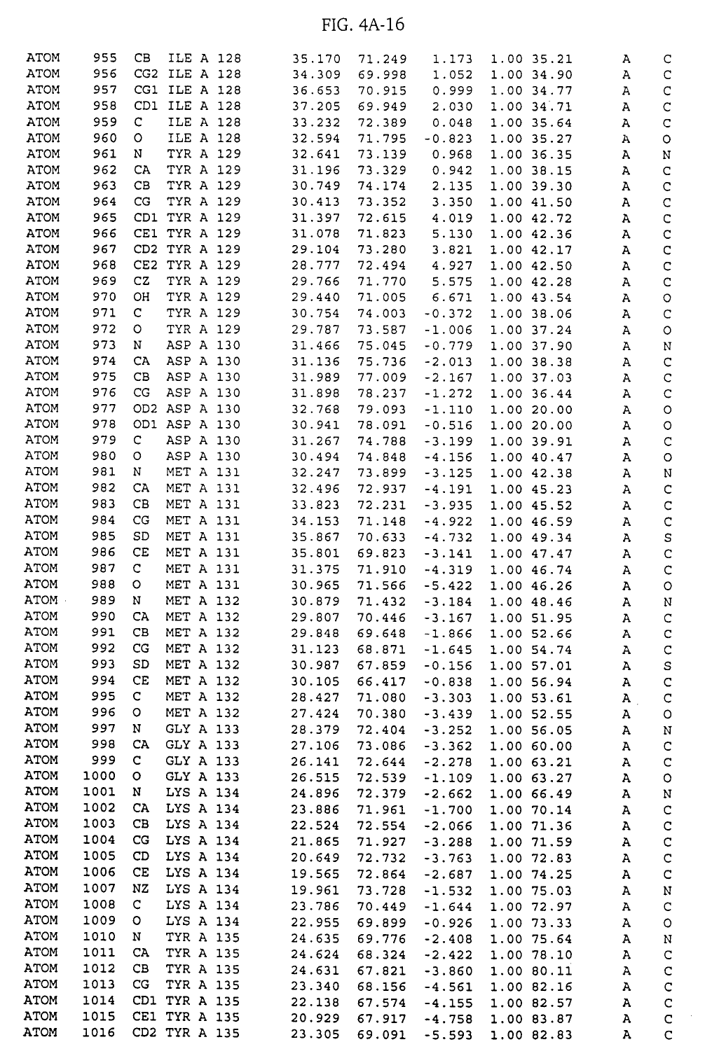

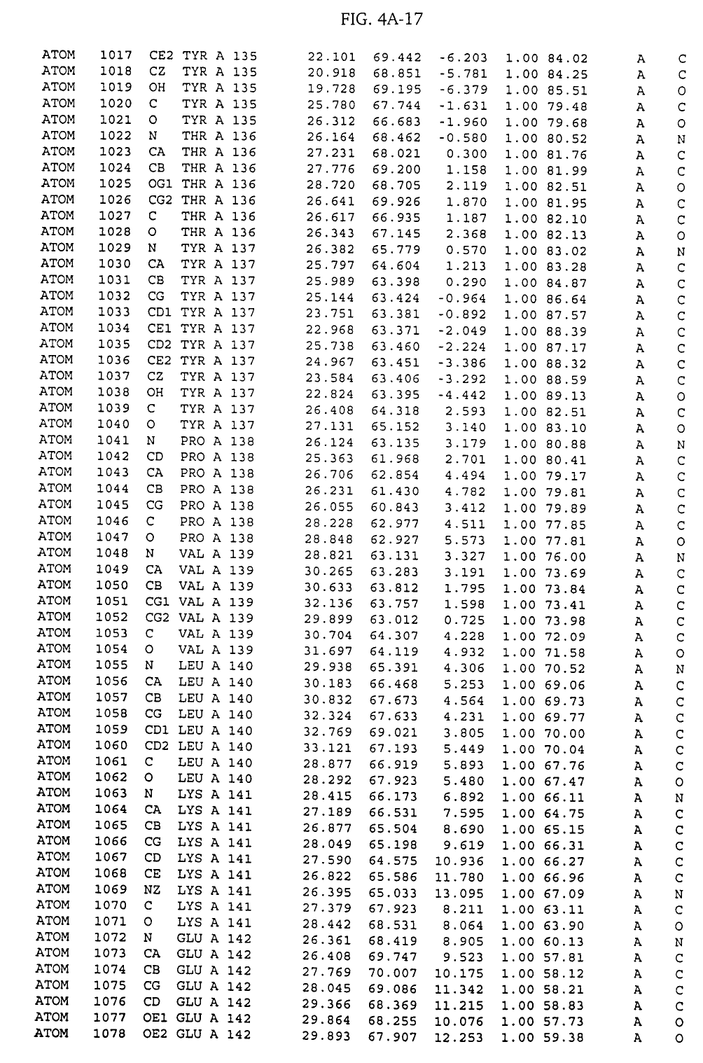

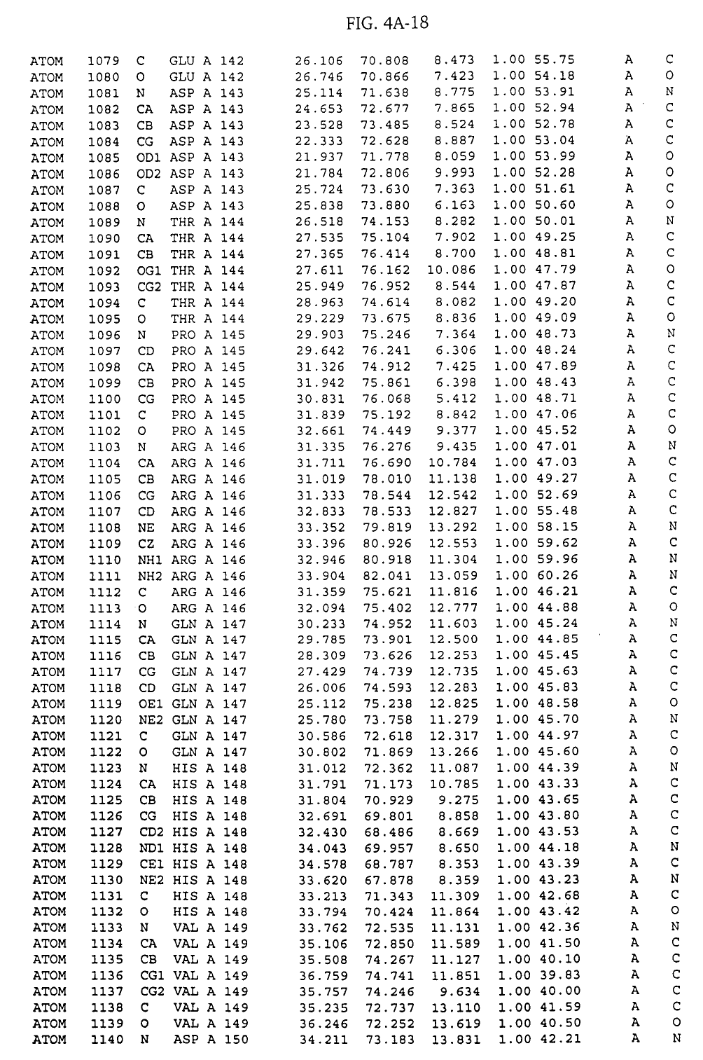

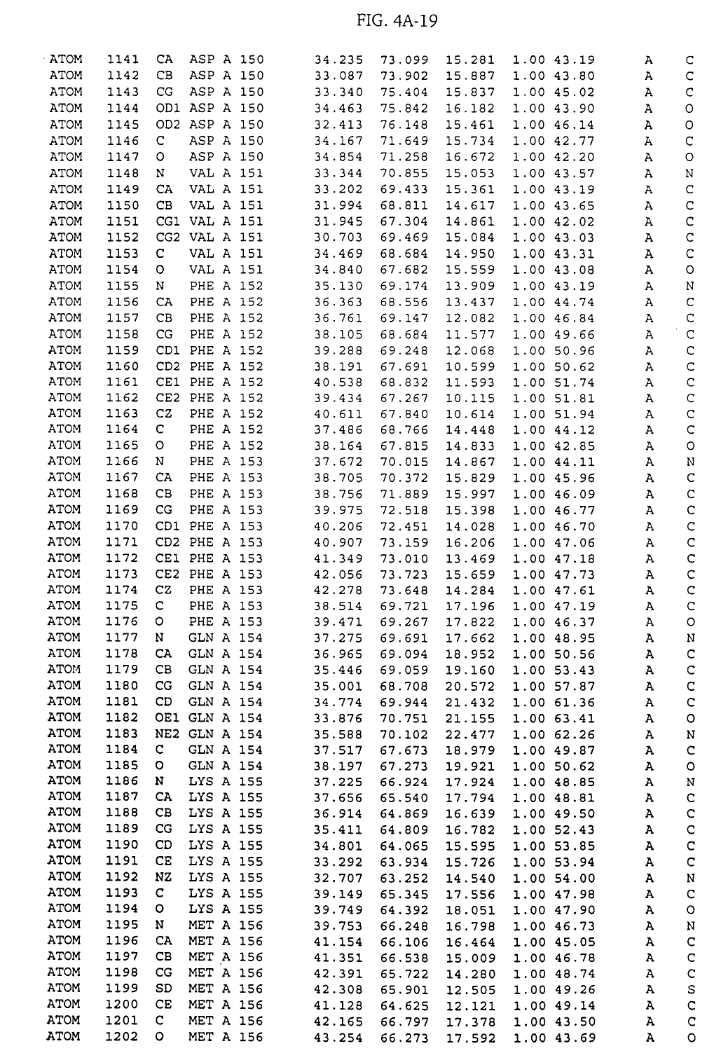

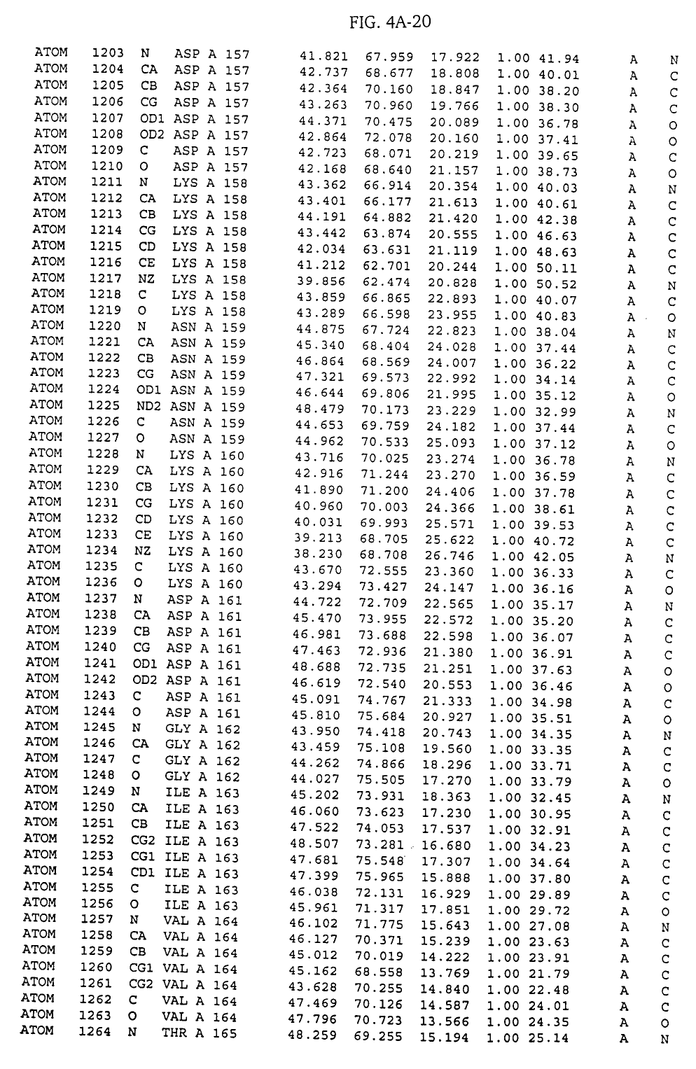

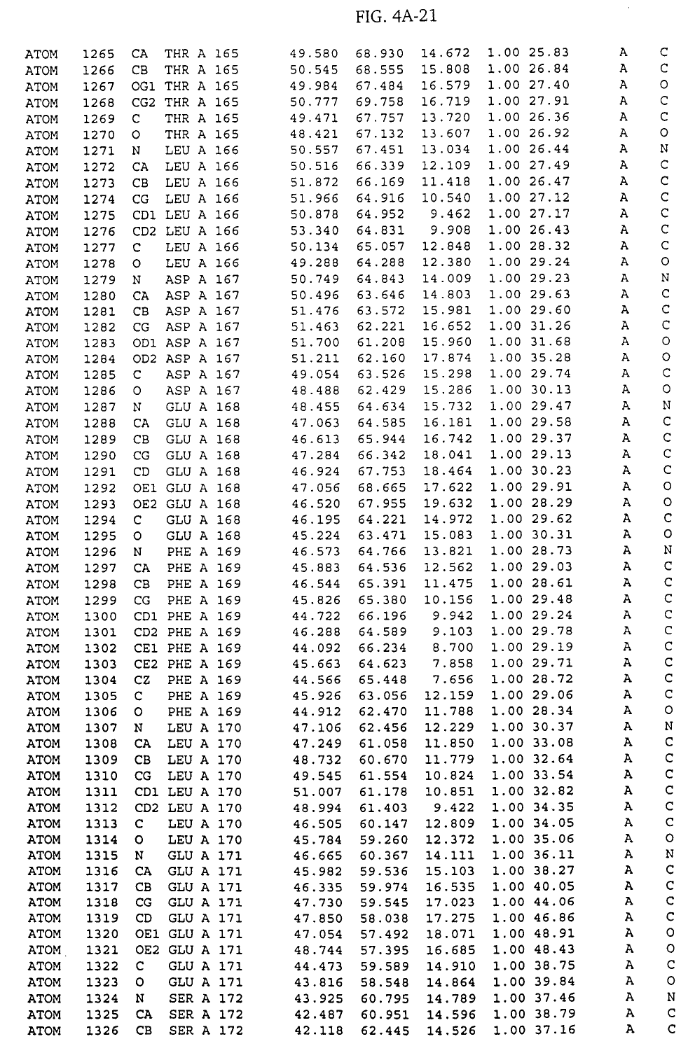

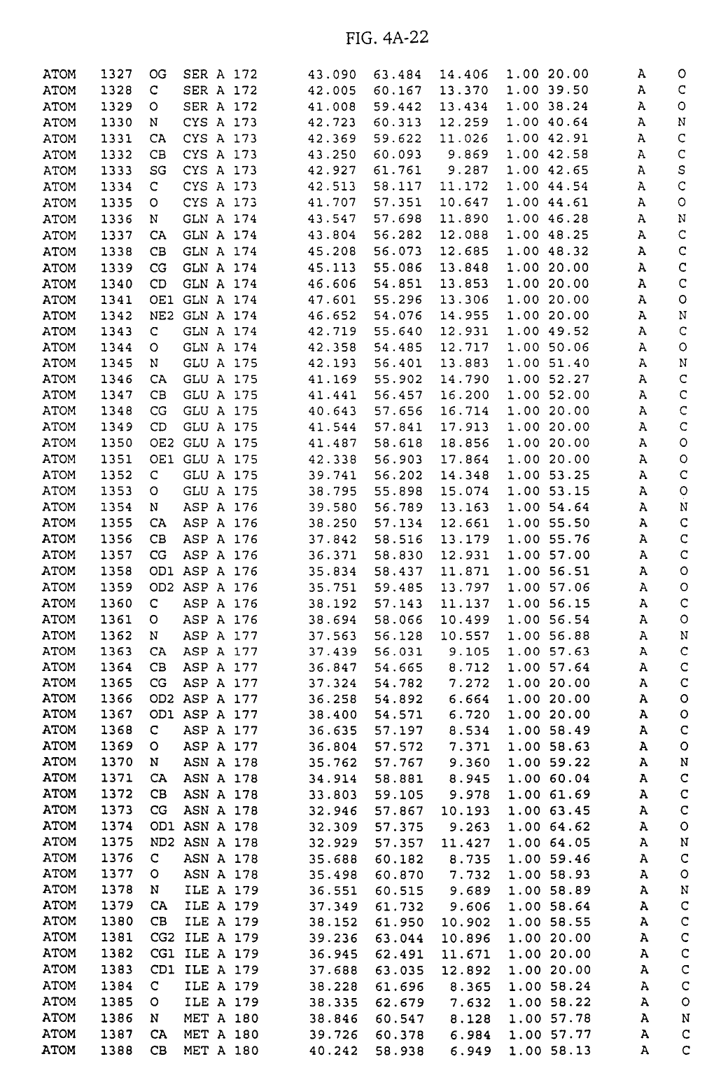

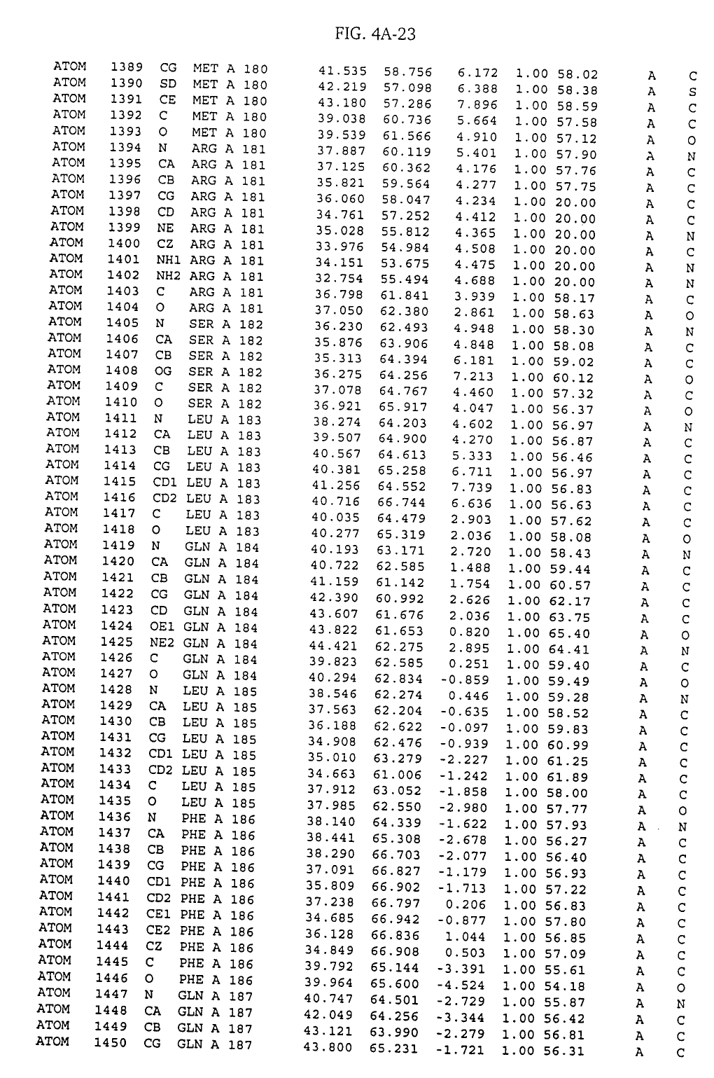

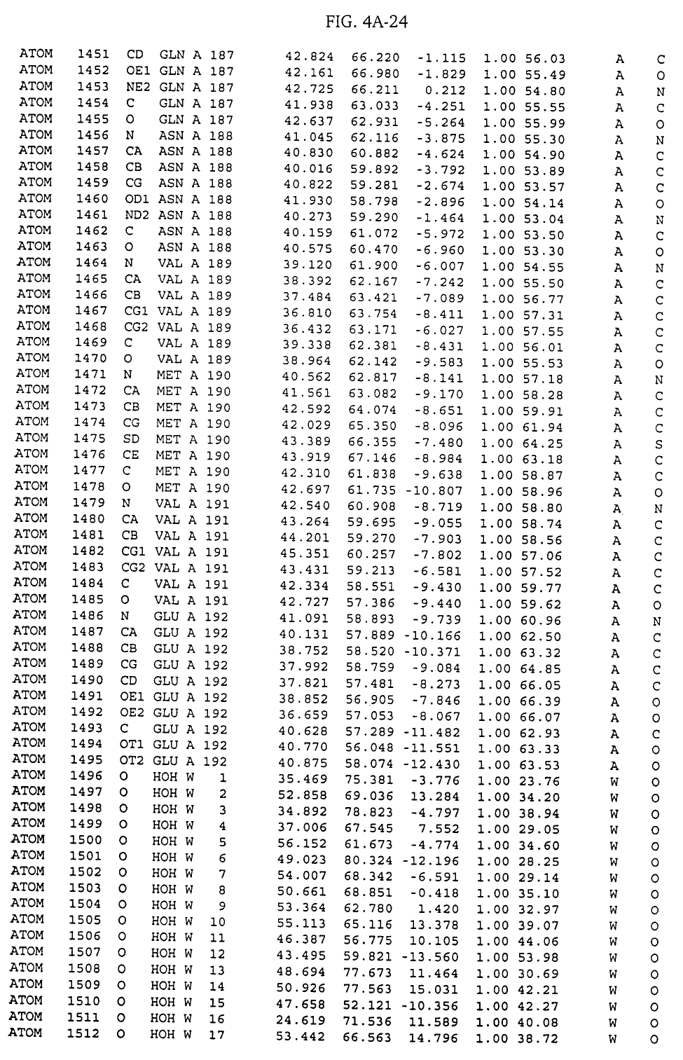

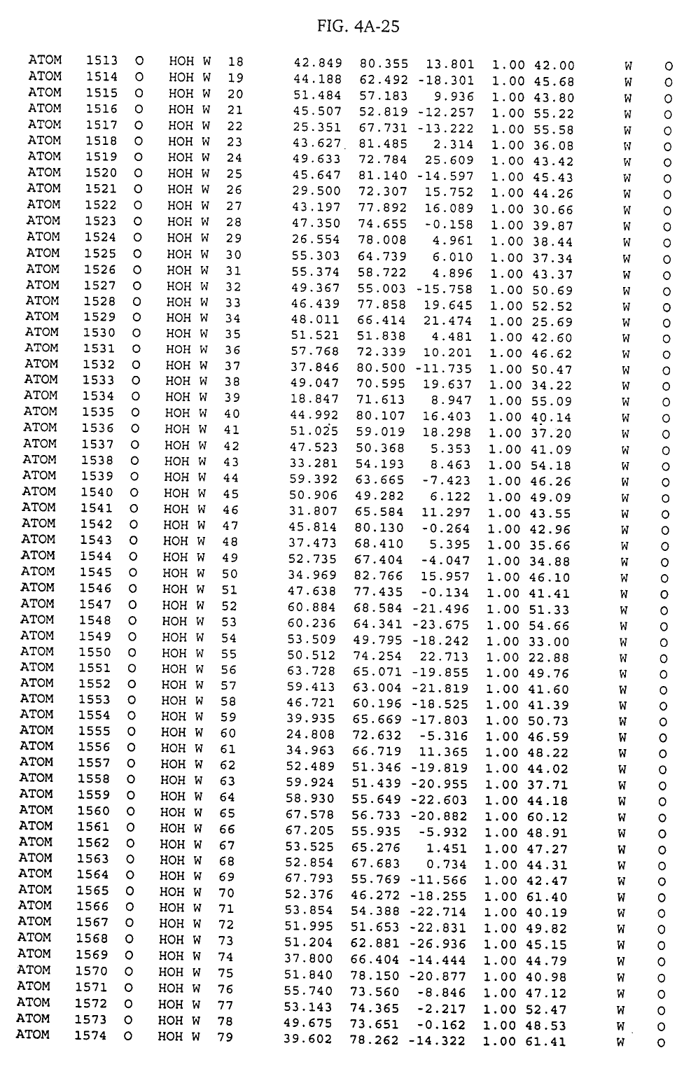

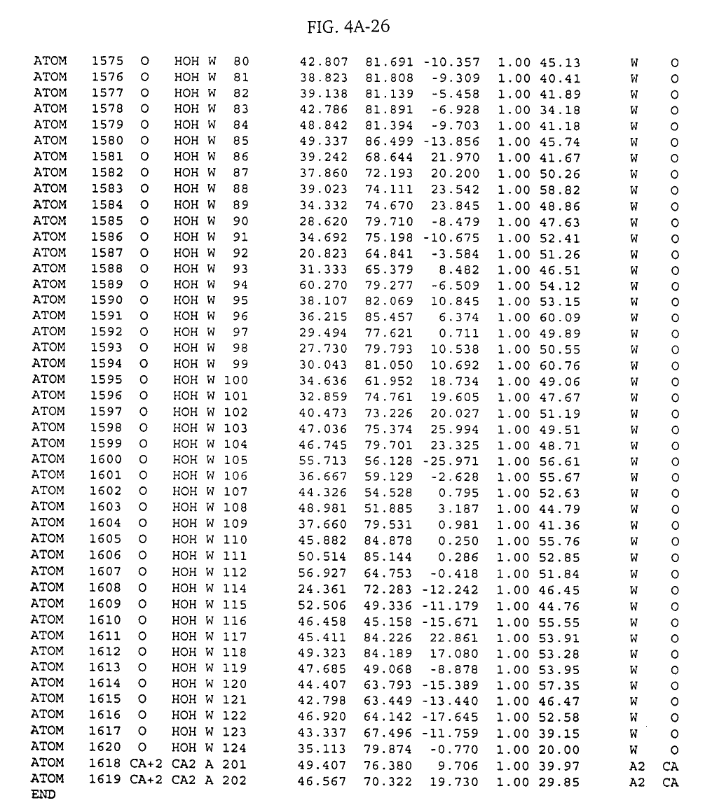

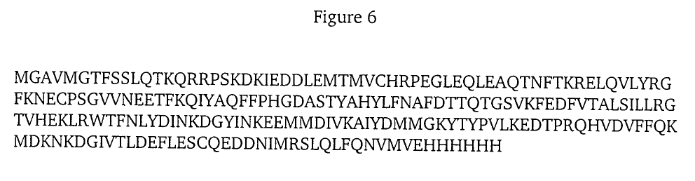

Unless otherwise noted, “KCHIP-1” is (i) Kv channel-interacting protein 1 having the amino acid sequence (residues 1-216) set forth in FIG. 6, including conservative substitutions thereof, (ii) modeled KCHIP-1 as defined by residues 12-192 of FIG. 4, including conservative substitutions thereof, or (iii) a KCHIP-1 analogue having a portion of amino acid residues 12-192 of FIG. 4 that define at least one active site or putative active site of KCHIP-1, including conservative substitutions thereof.

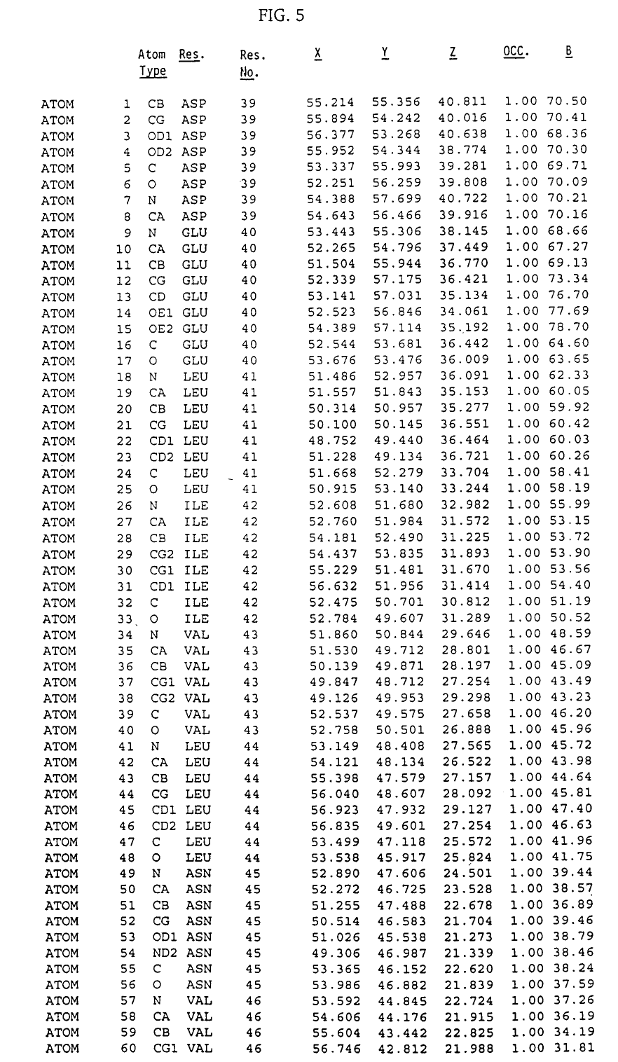

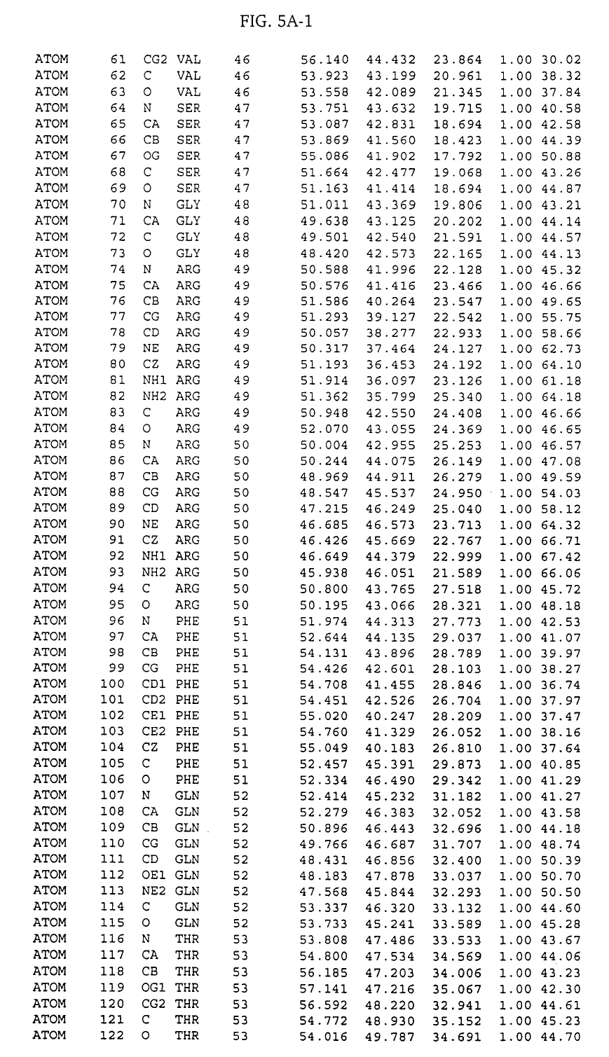

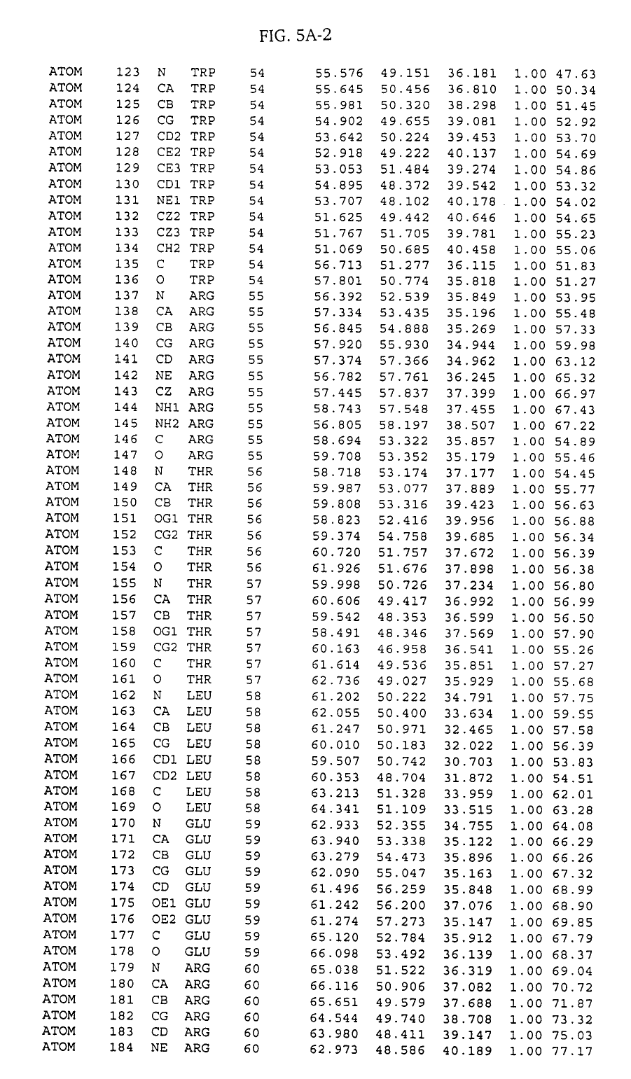

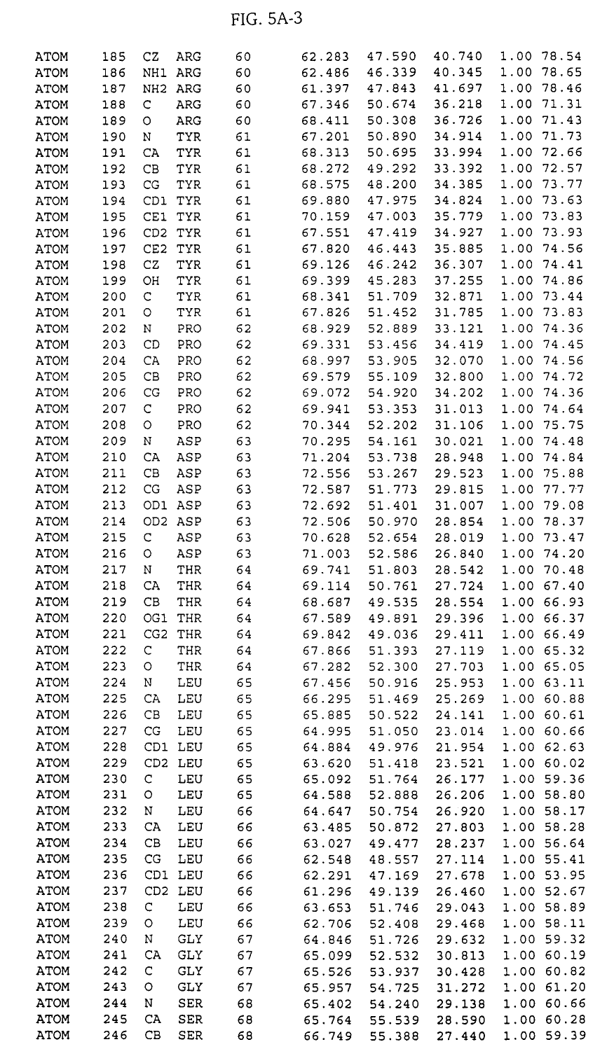

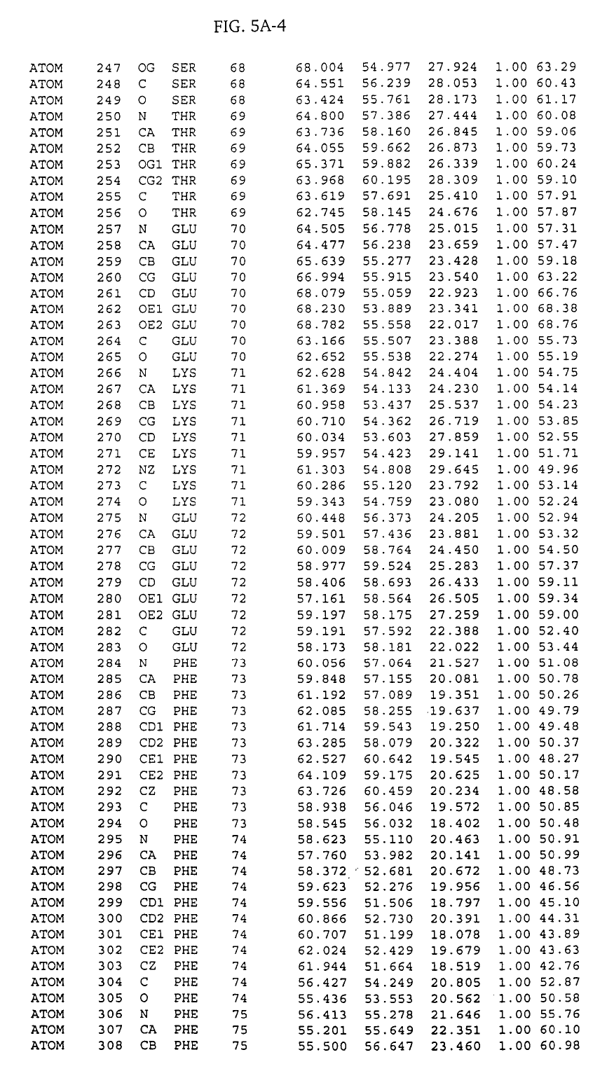

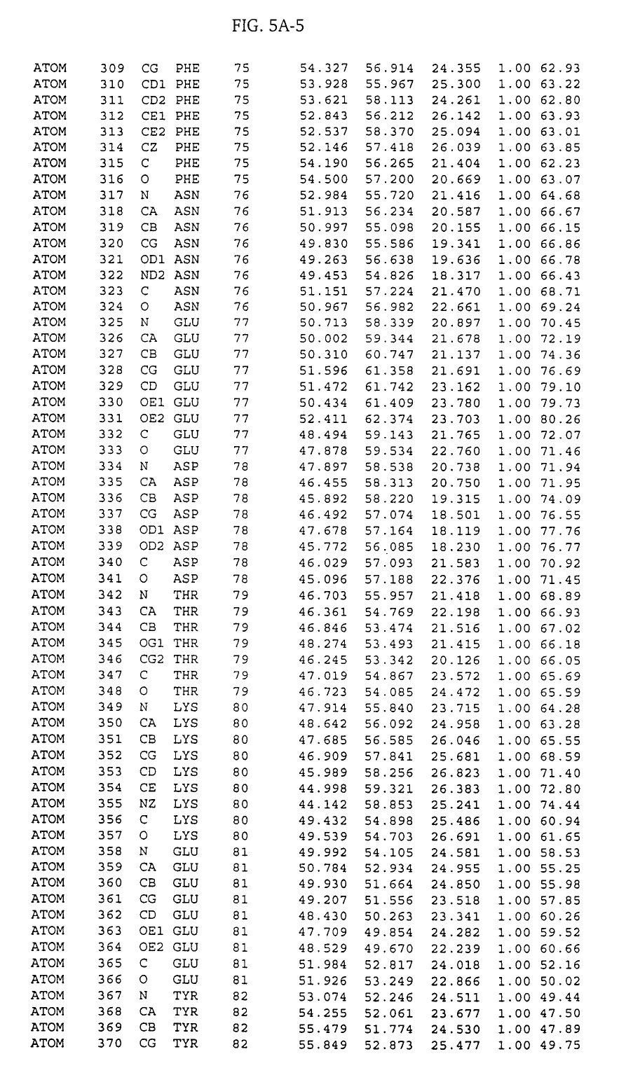

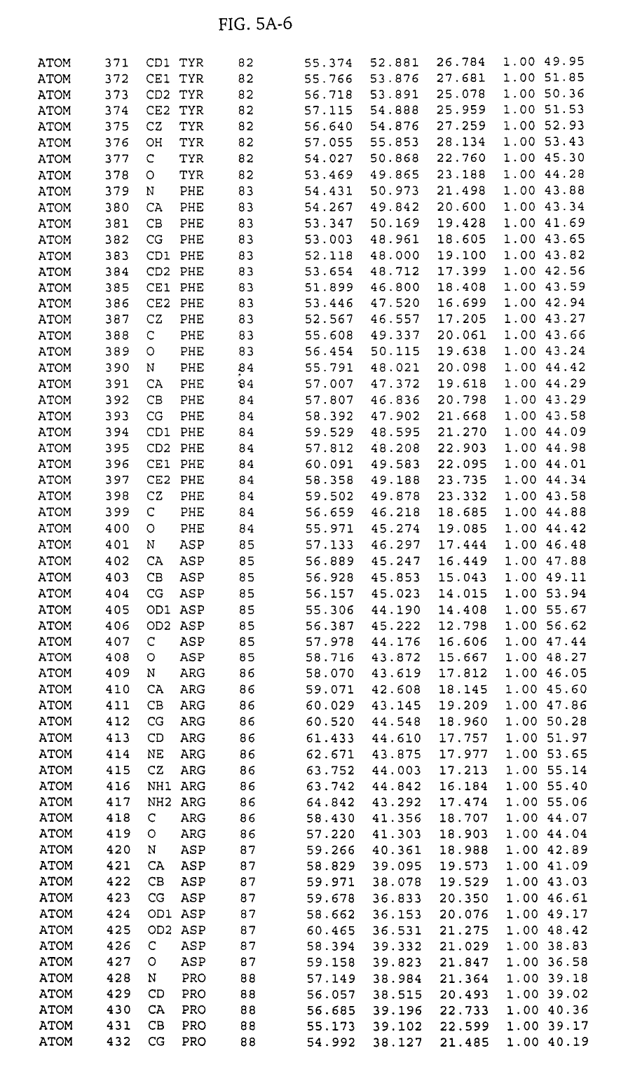

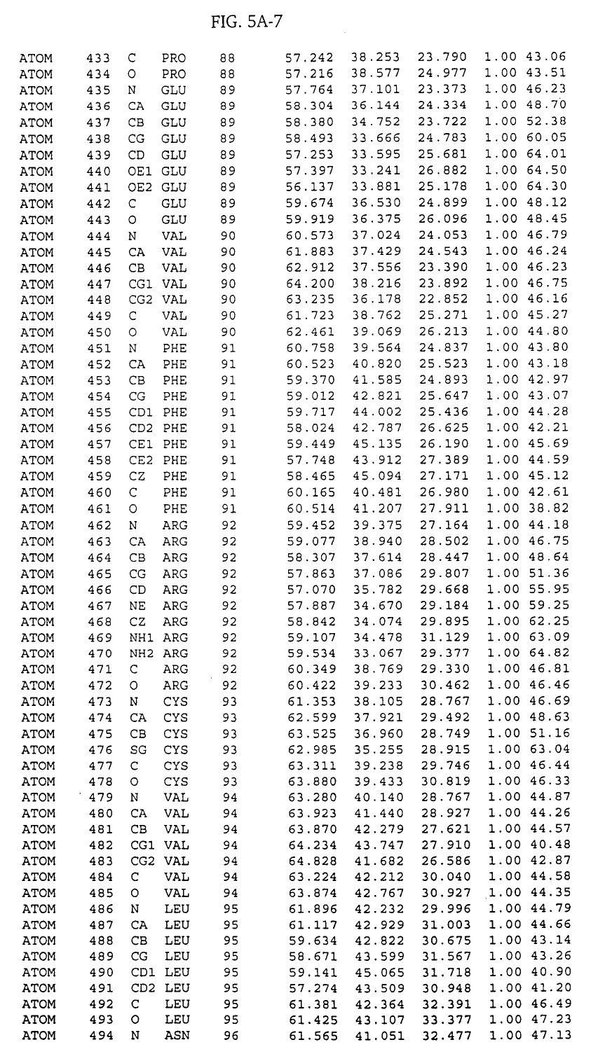

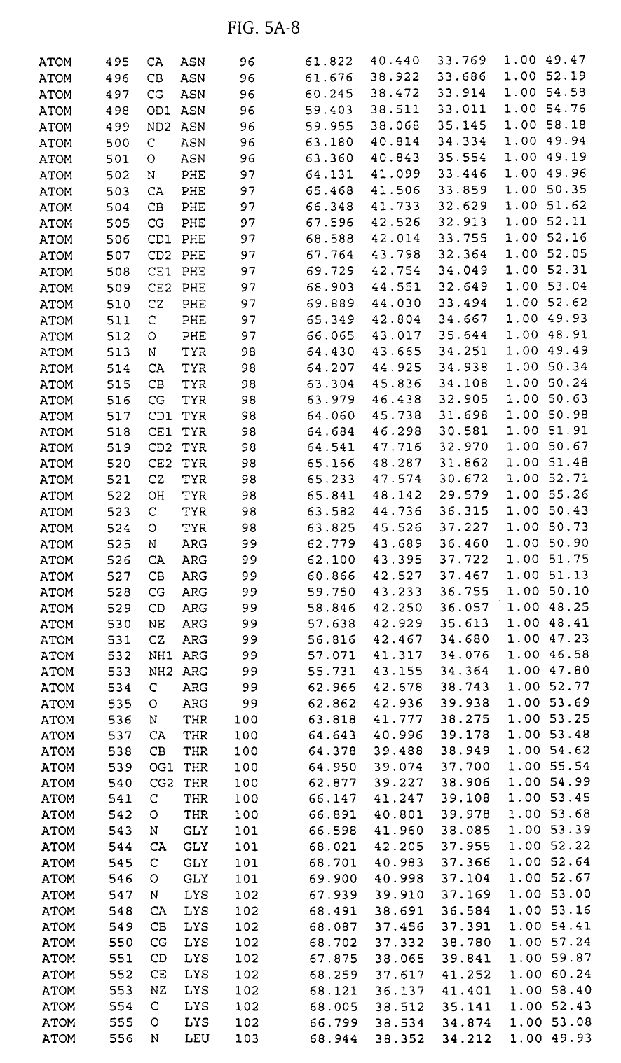

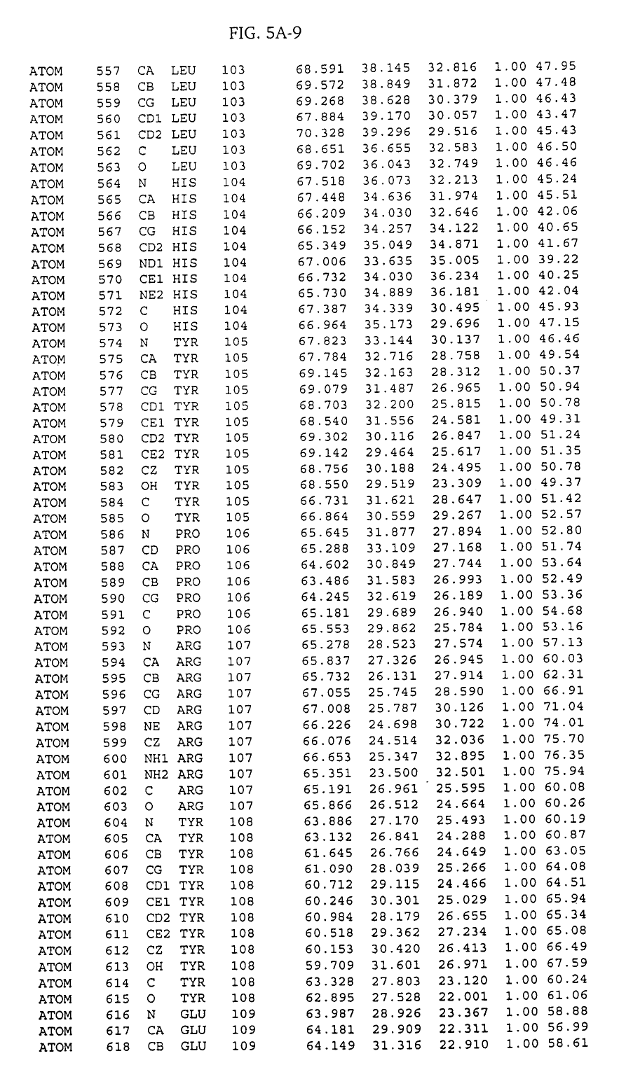

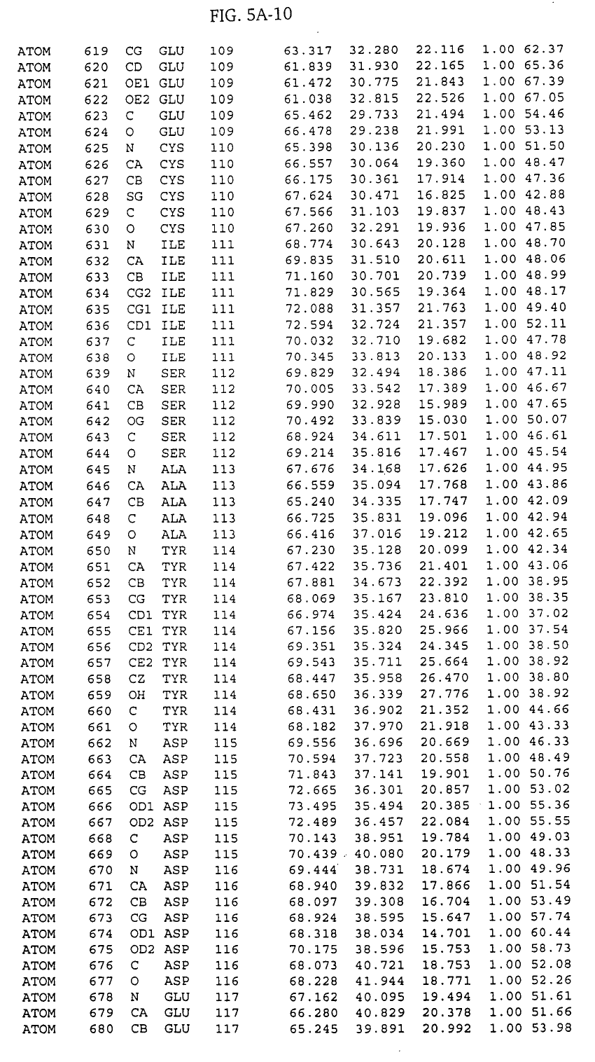

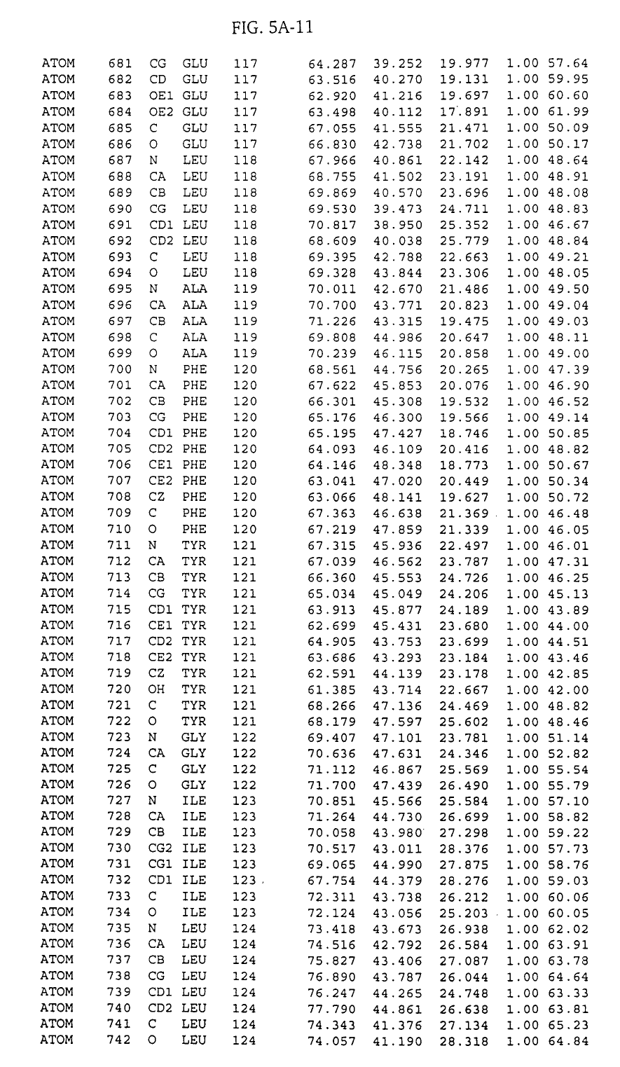

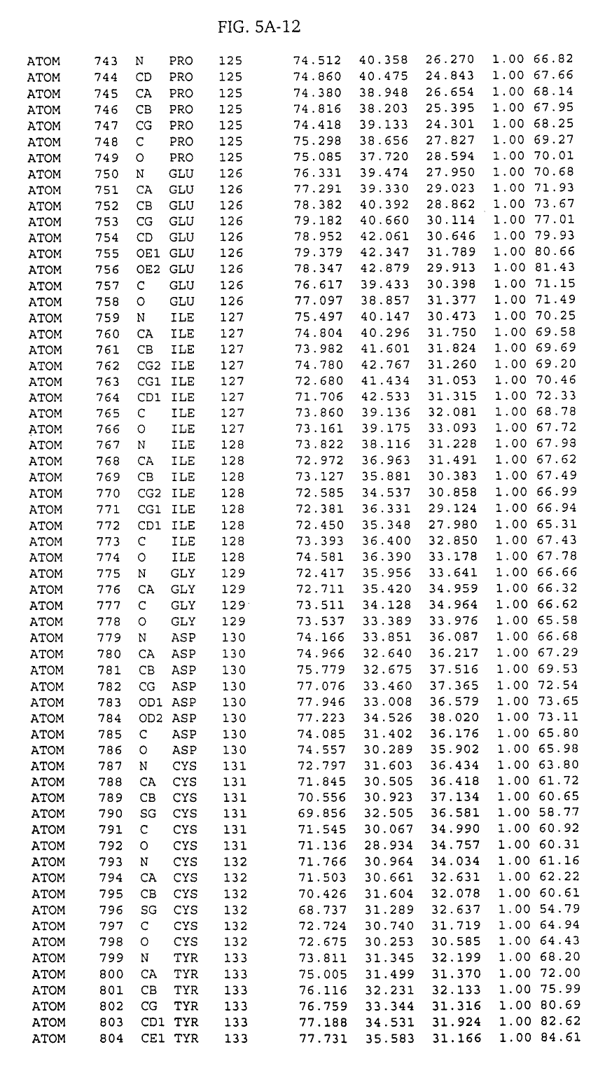

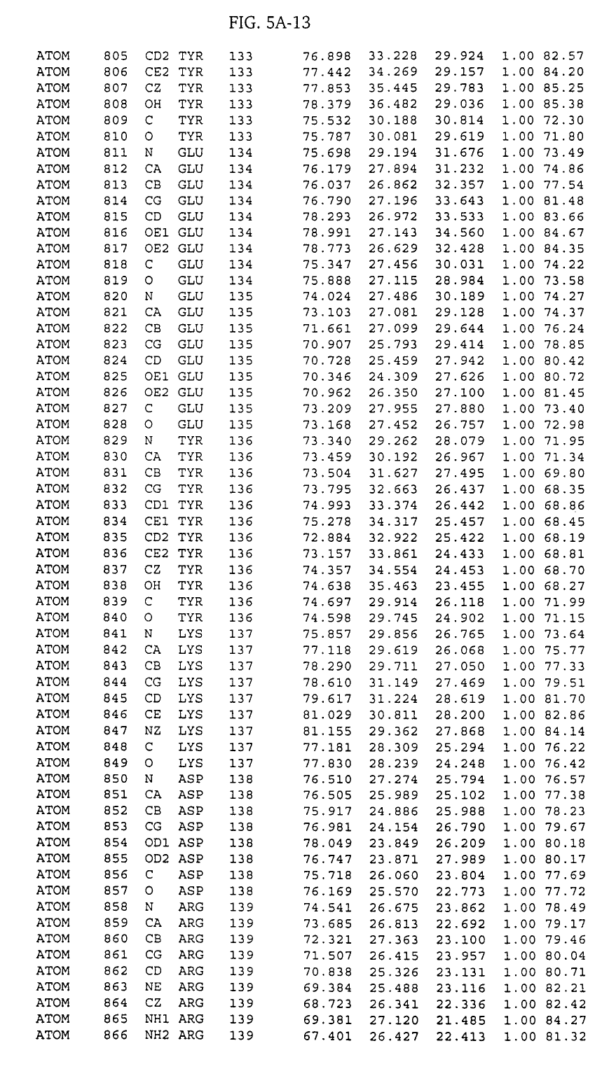

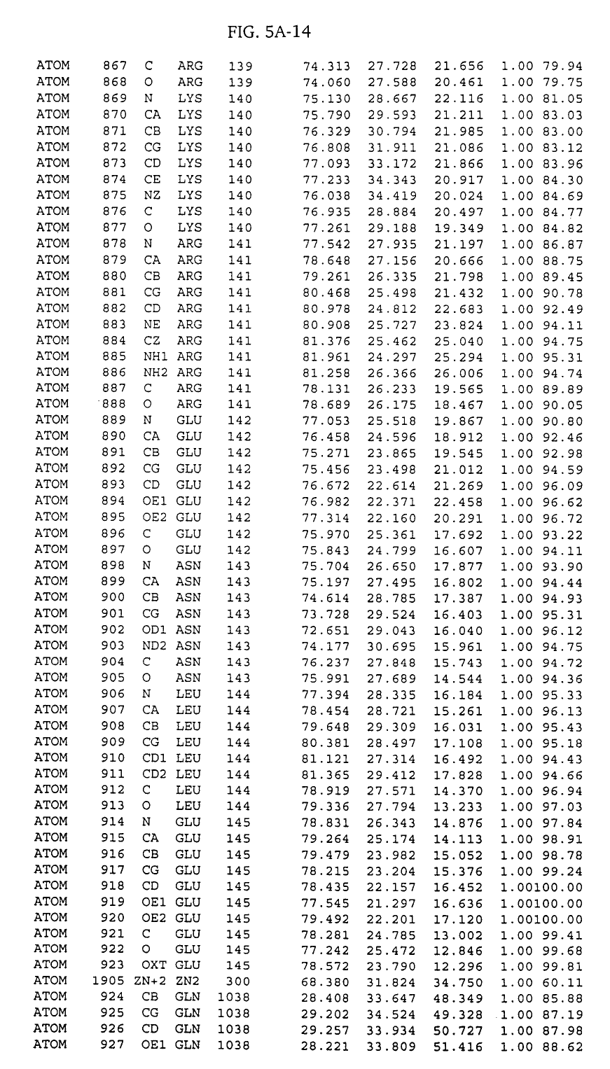

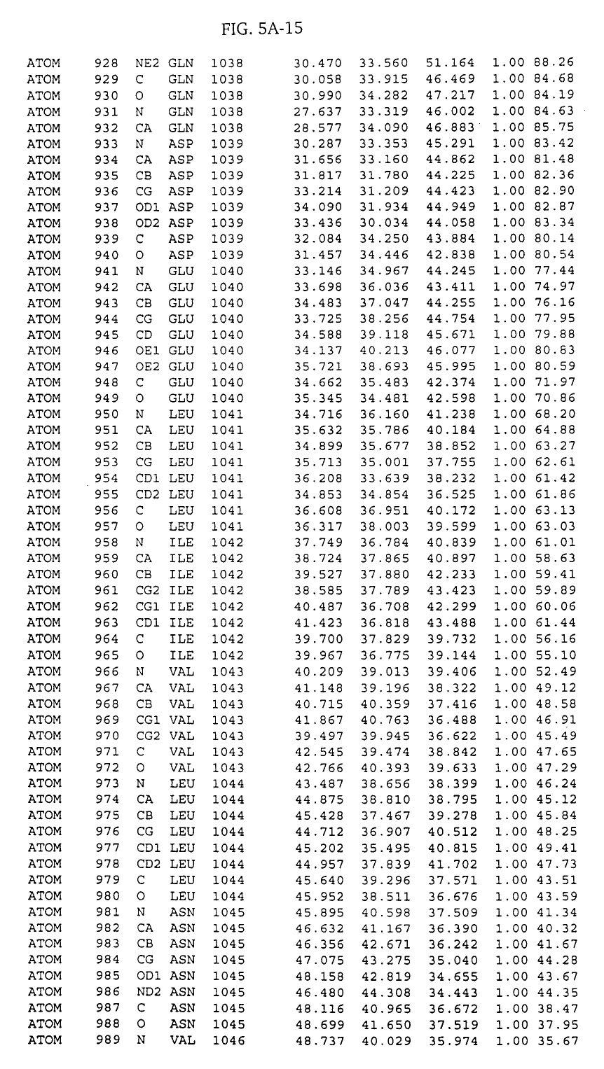

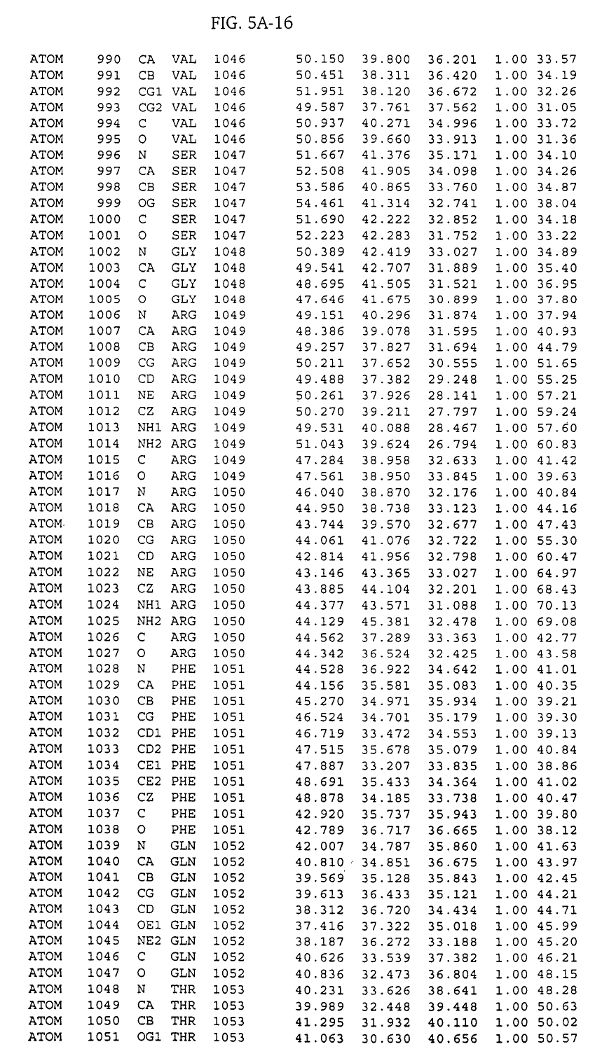

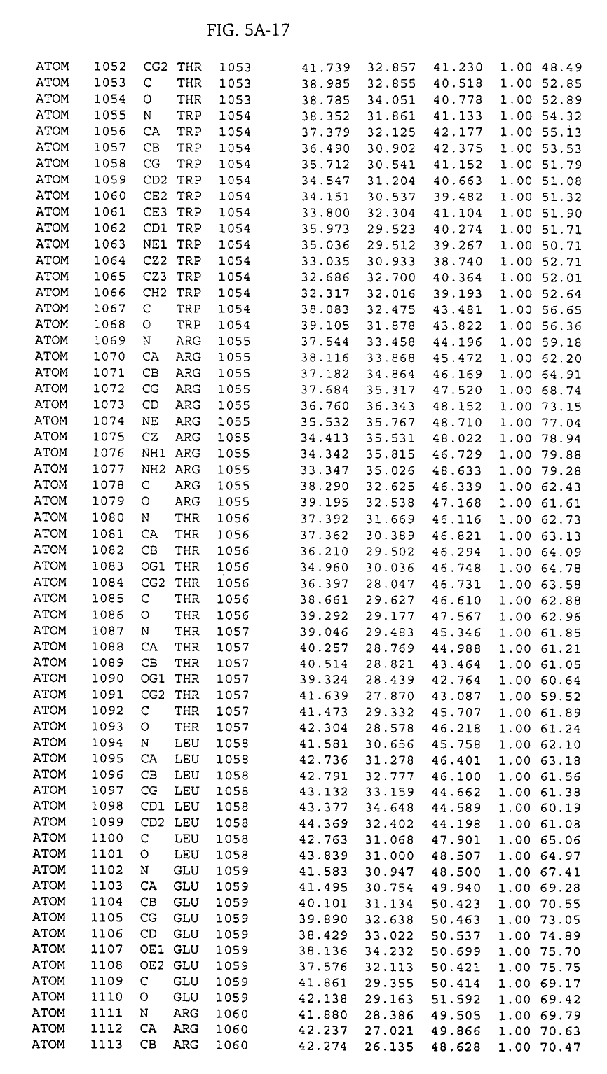

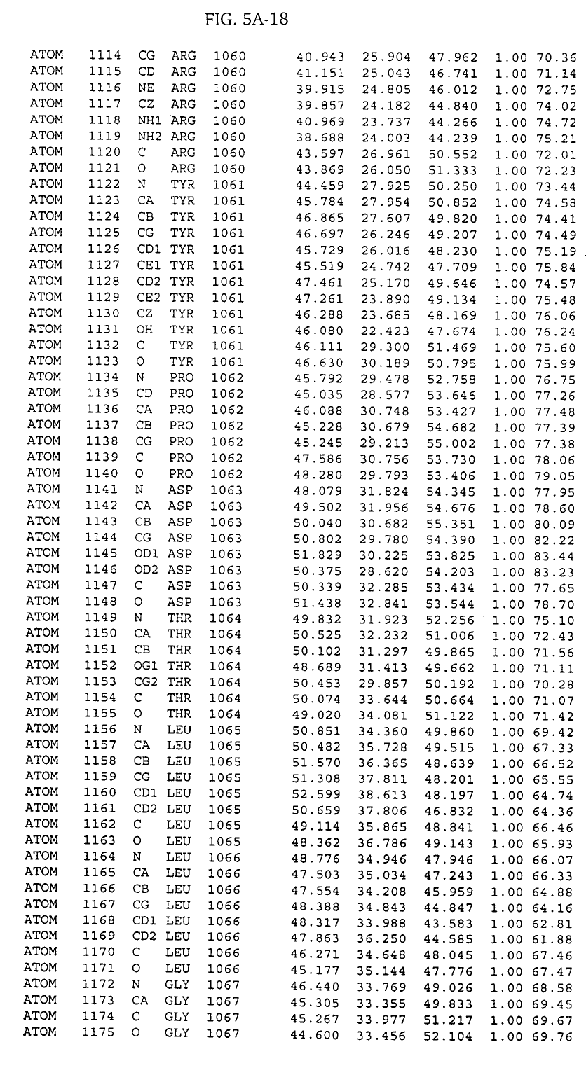

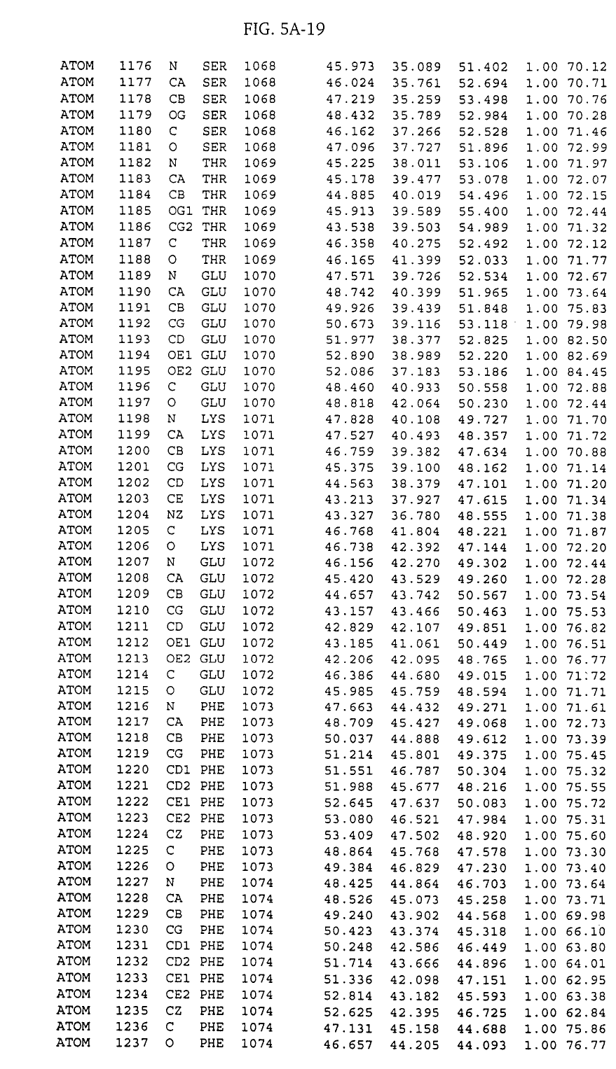

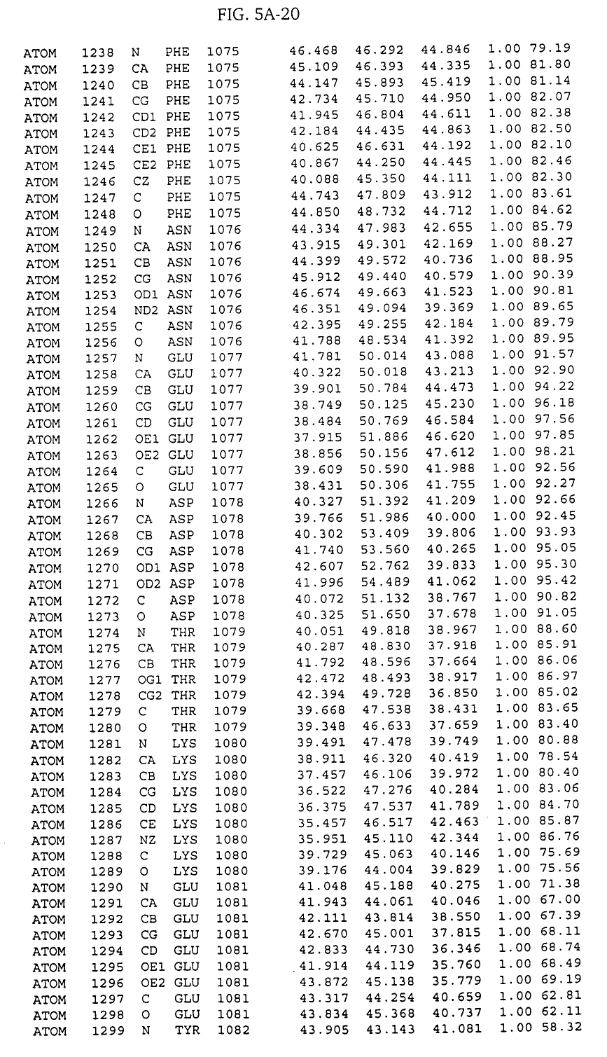

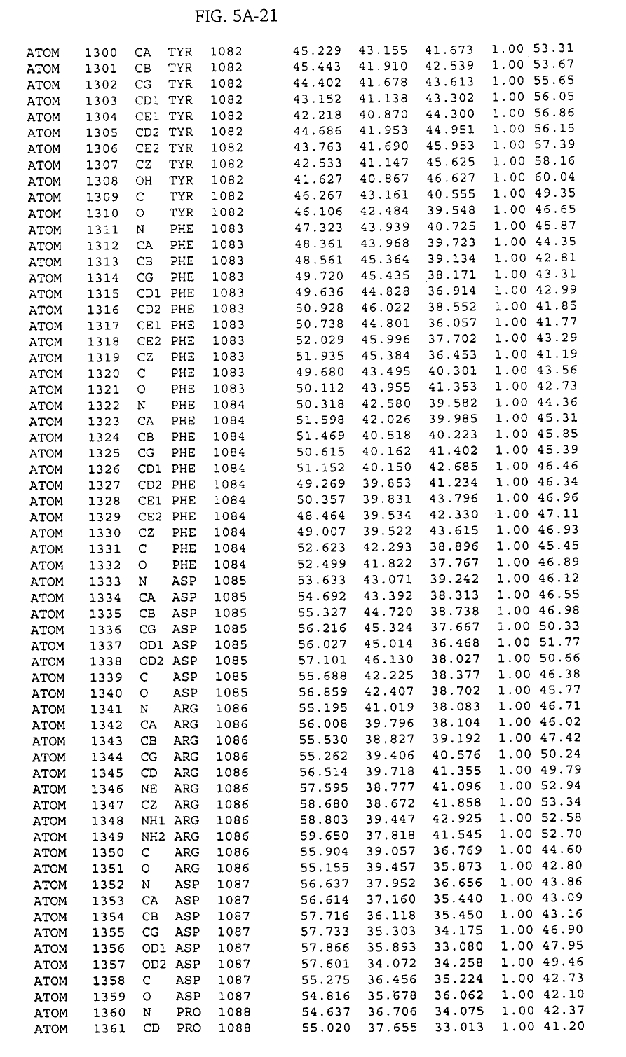

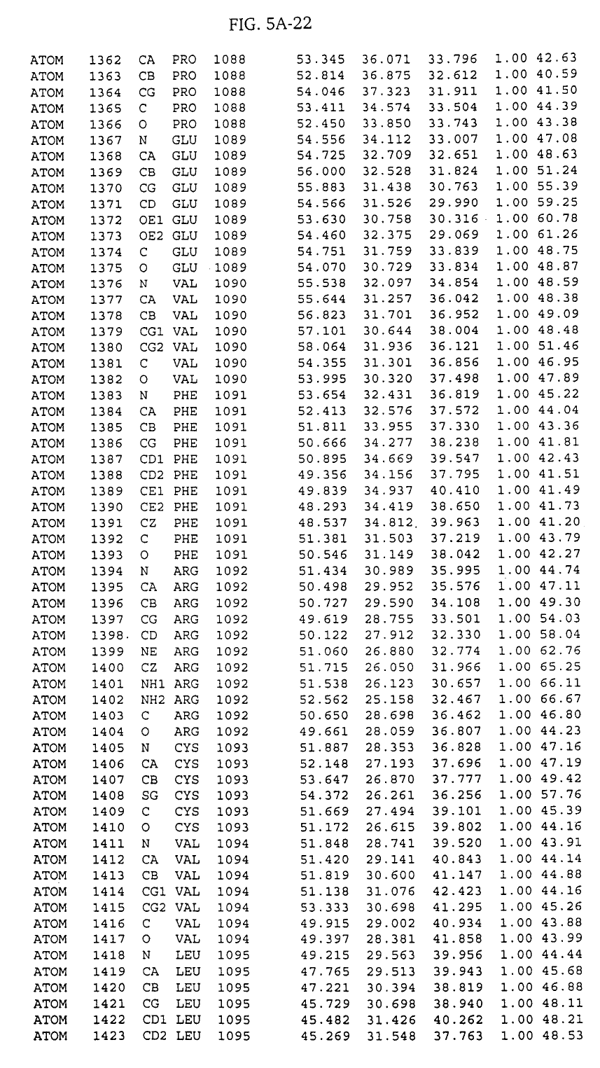

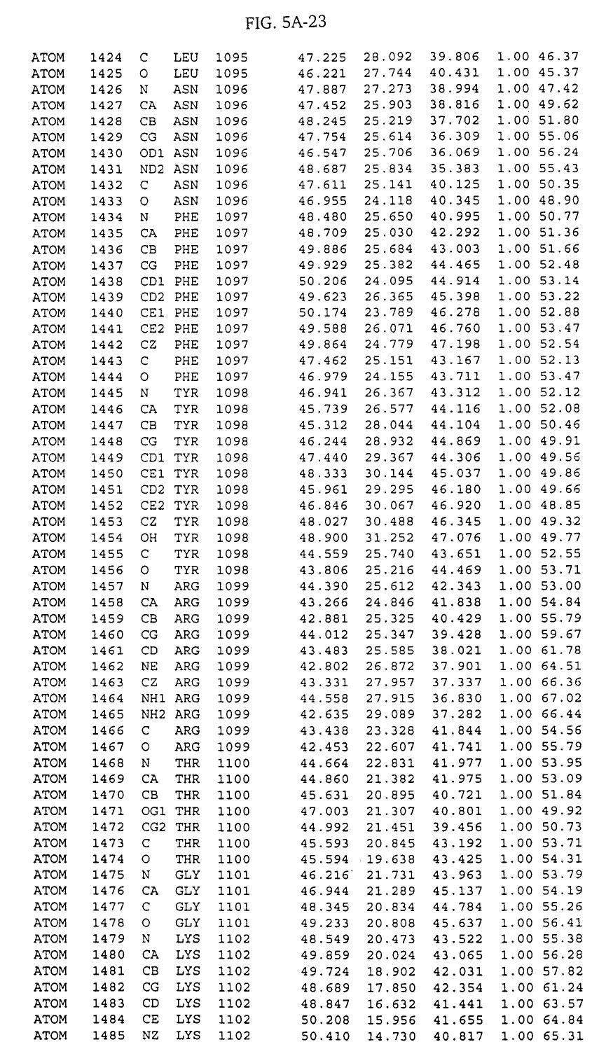

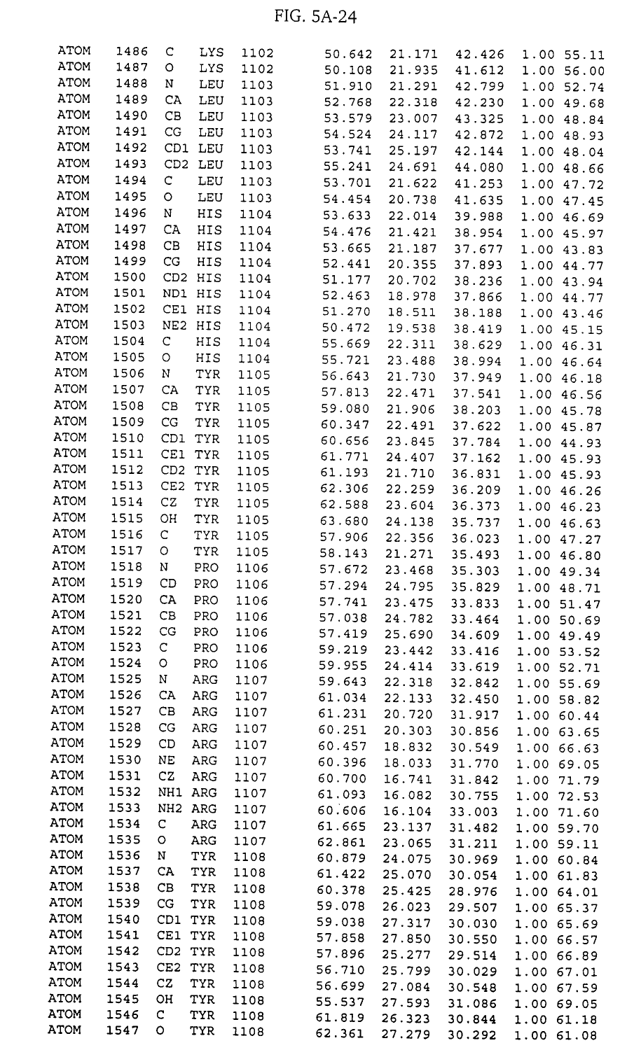

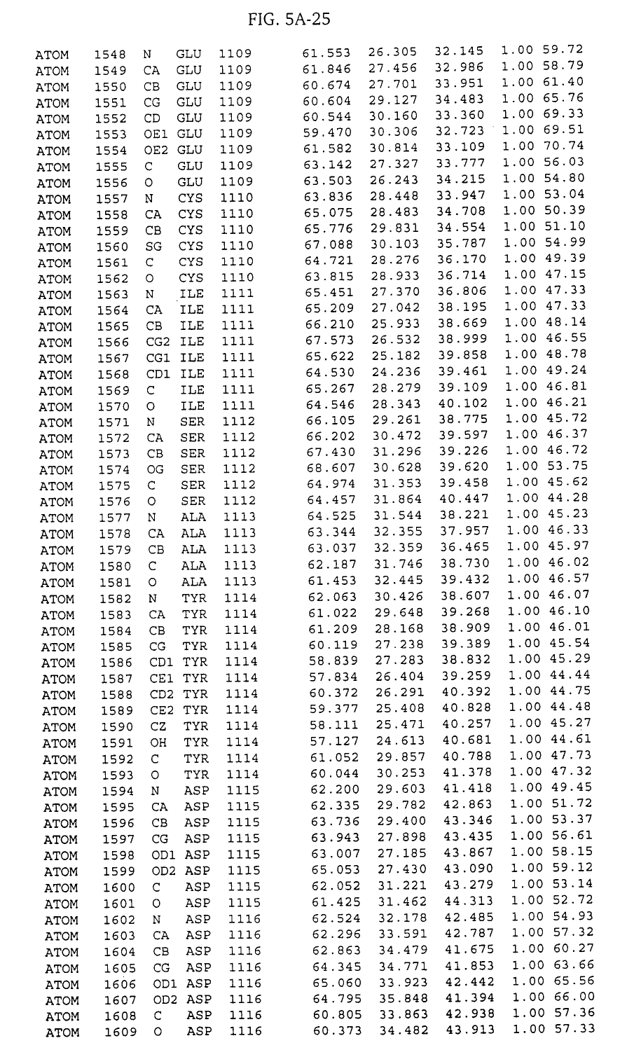

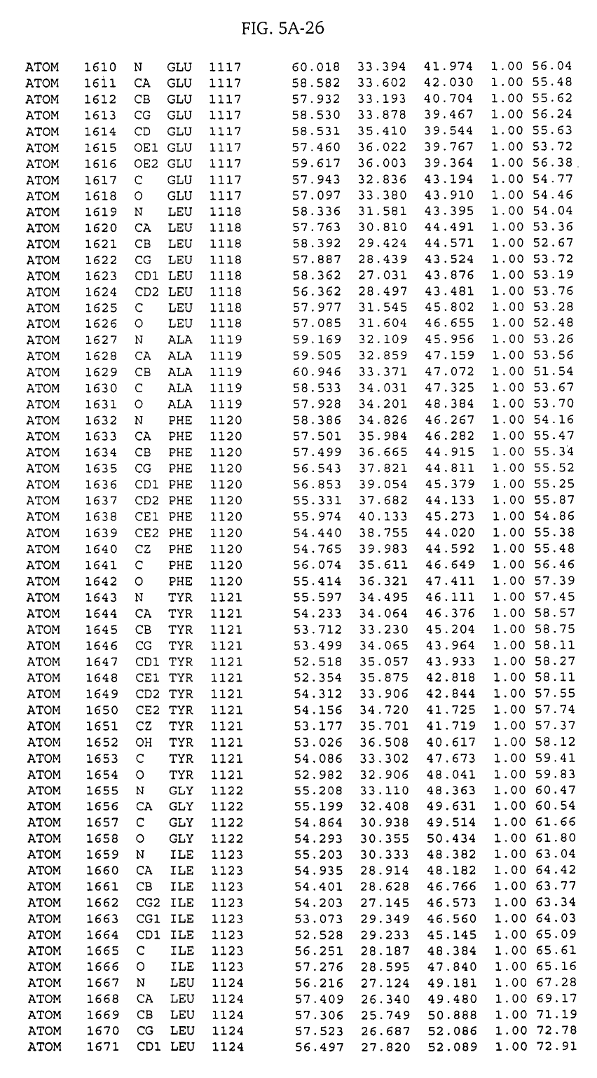

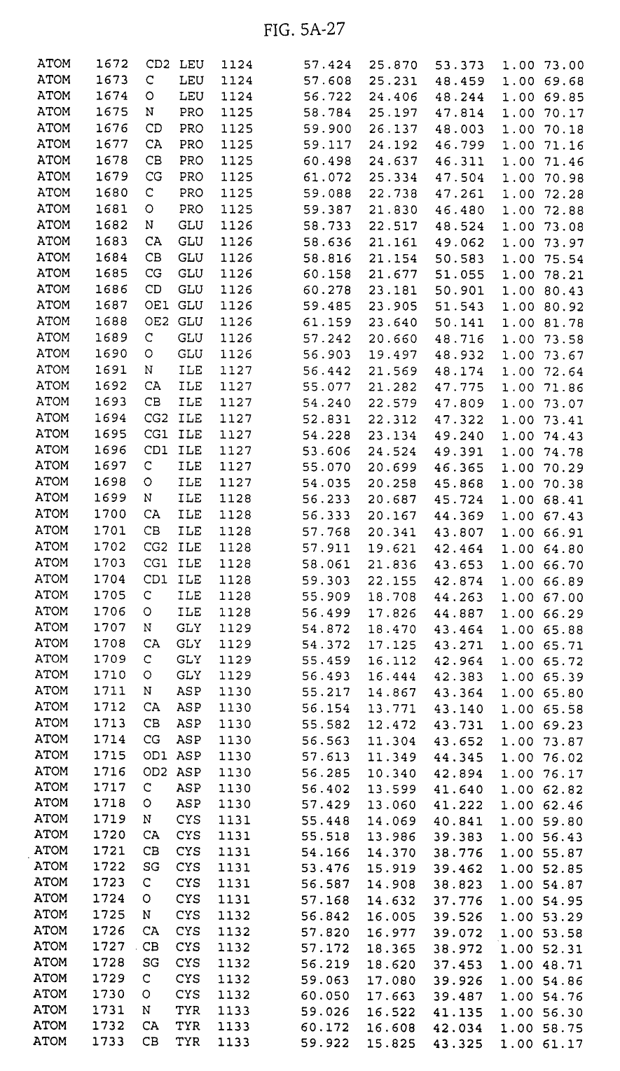

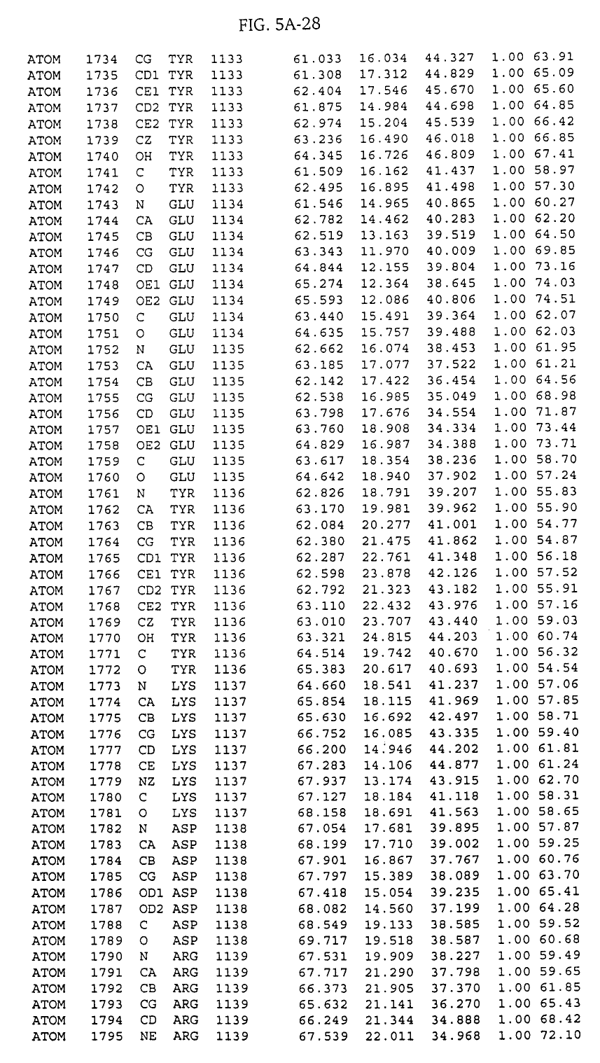

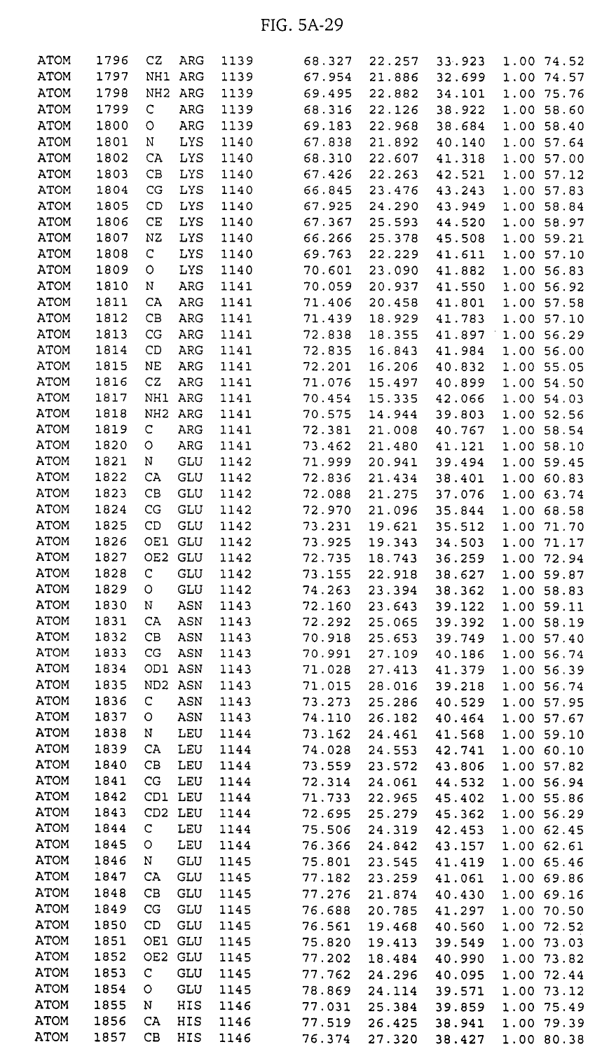

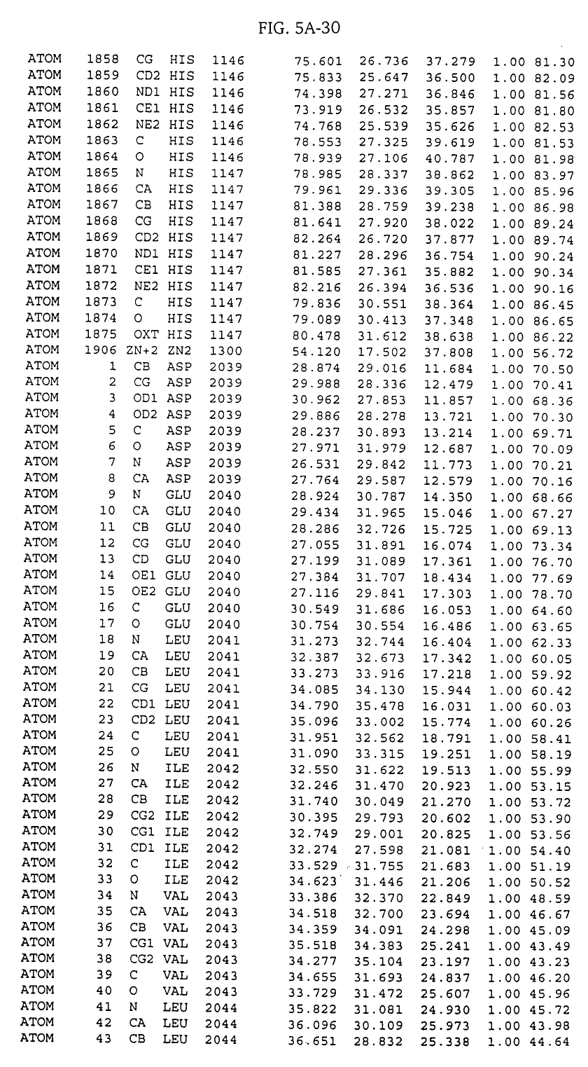

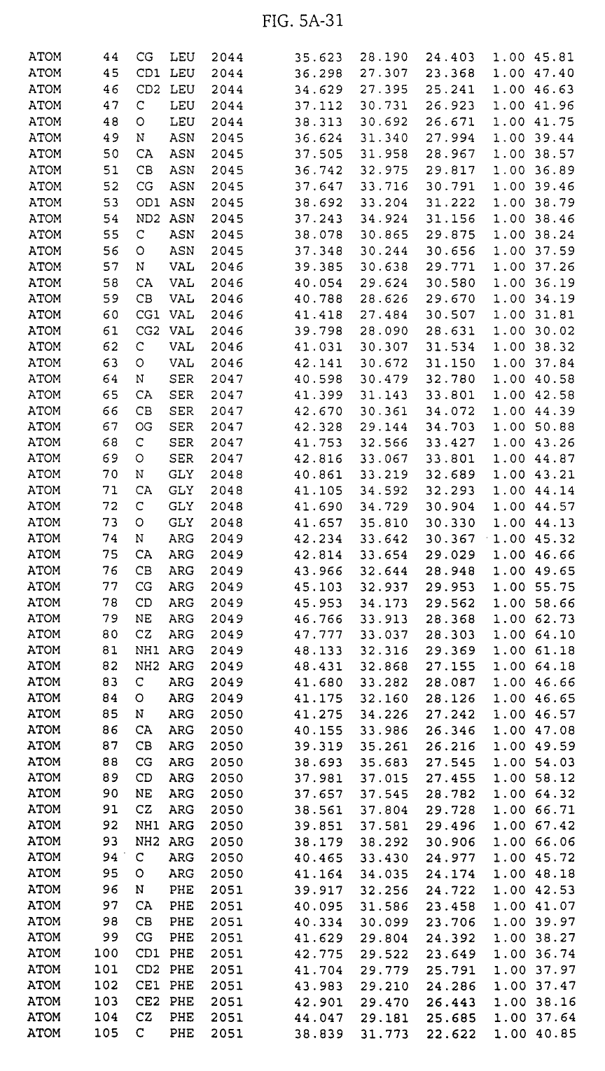

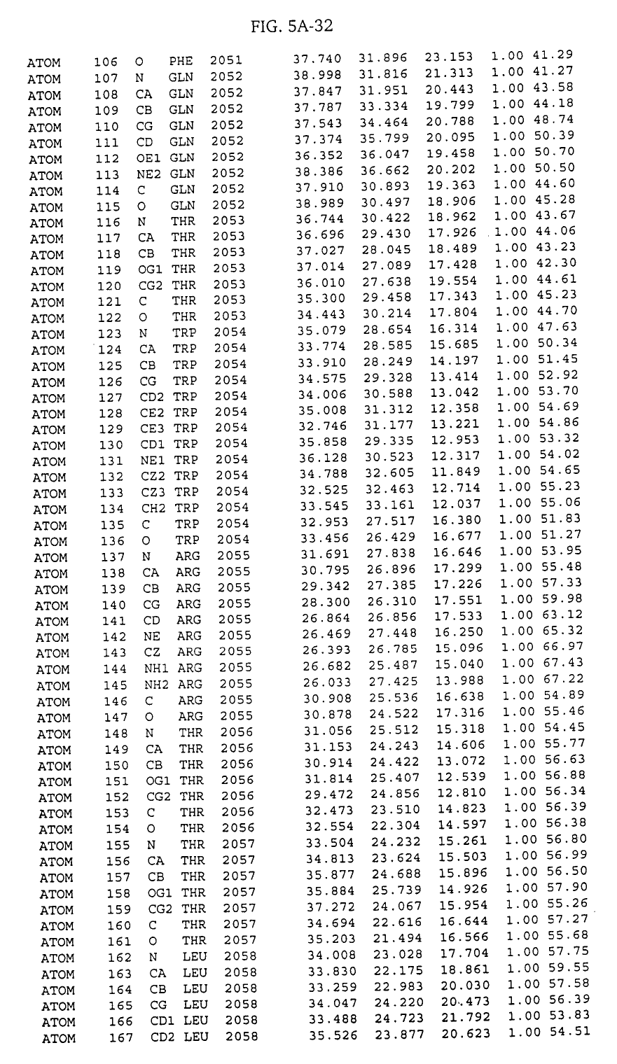

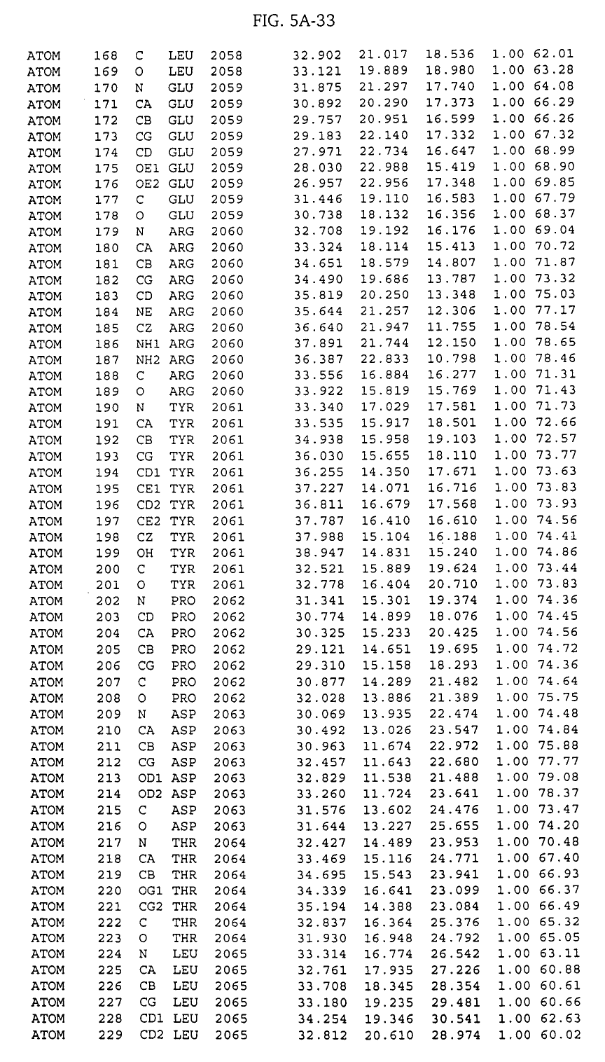

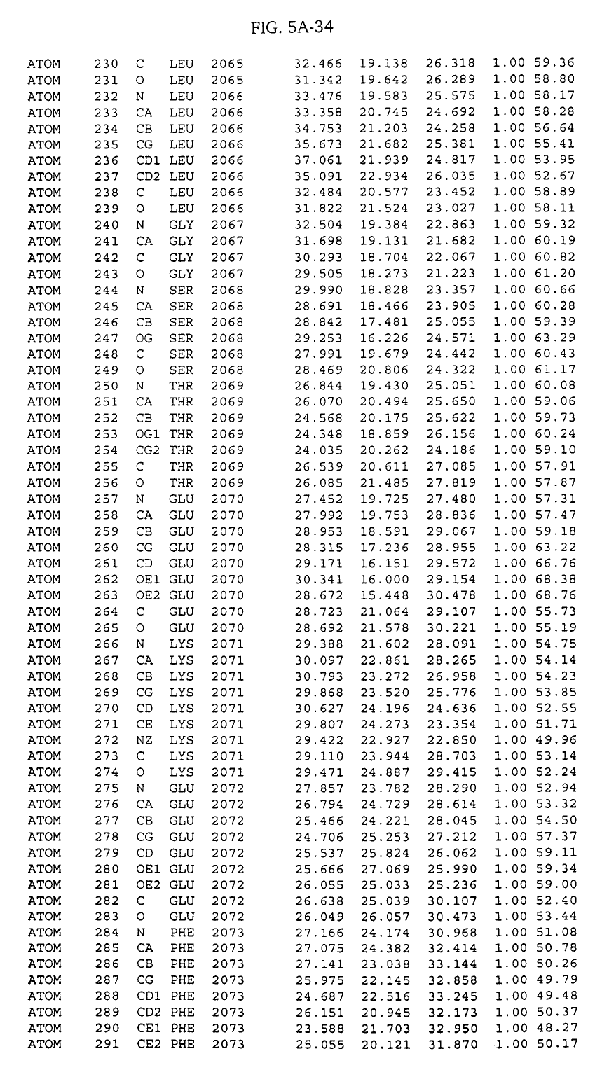

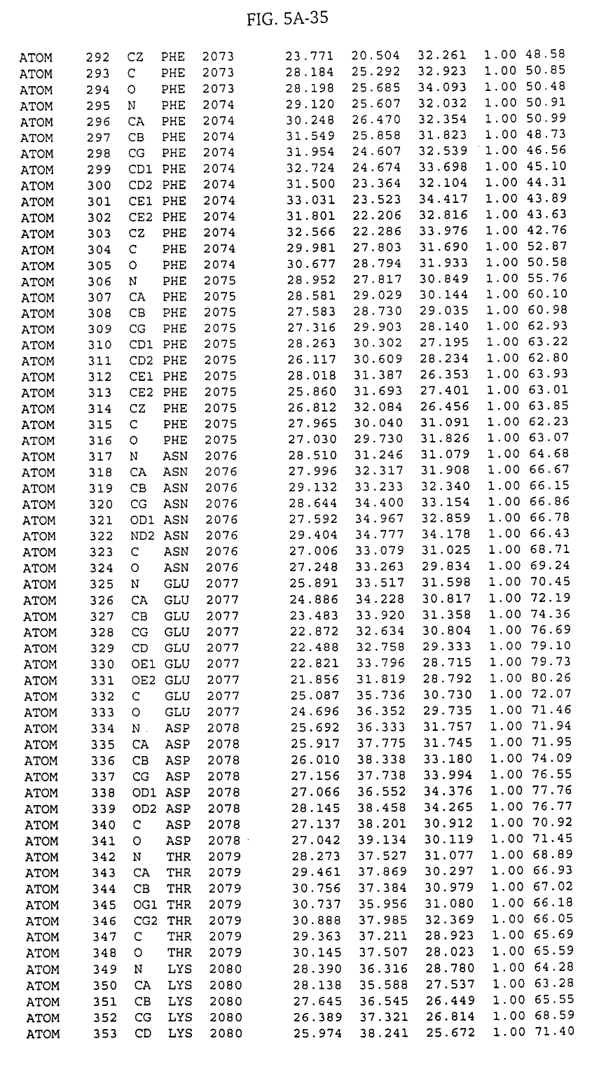

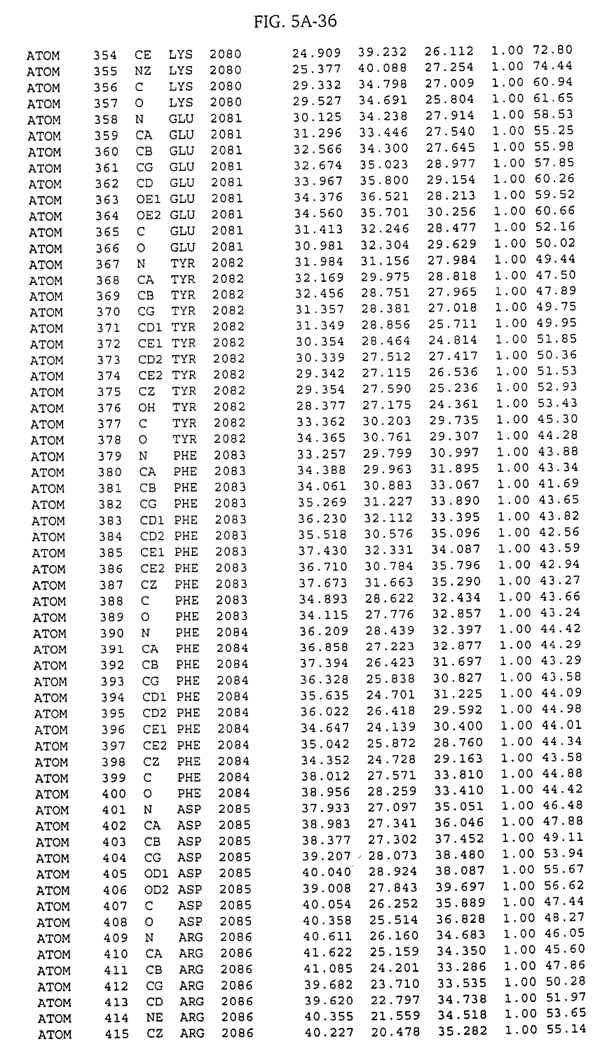

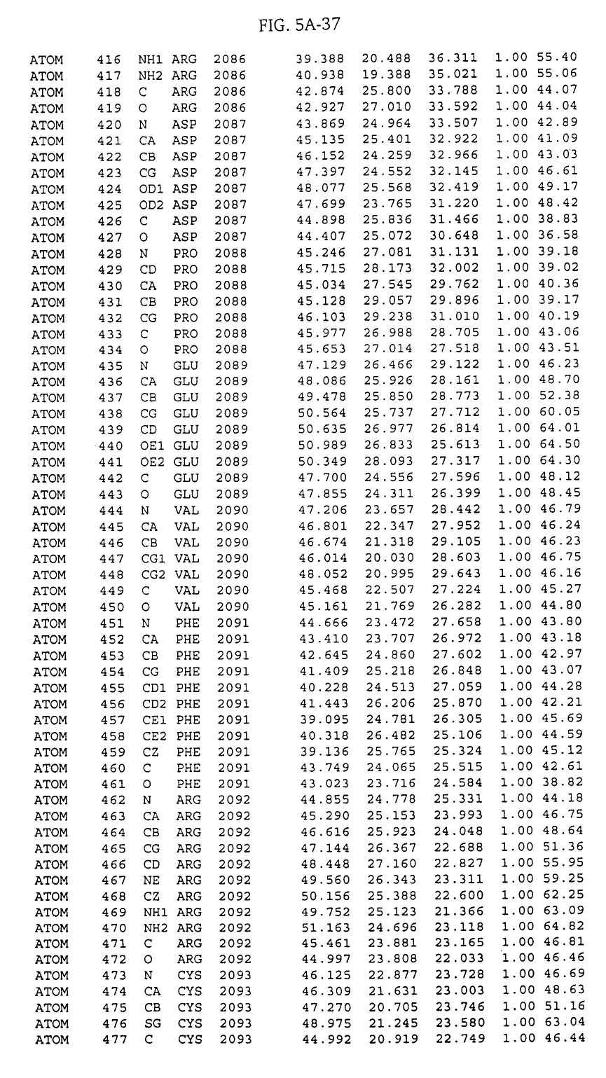

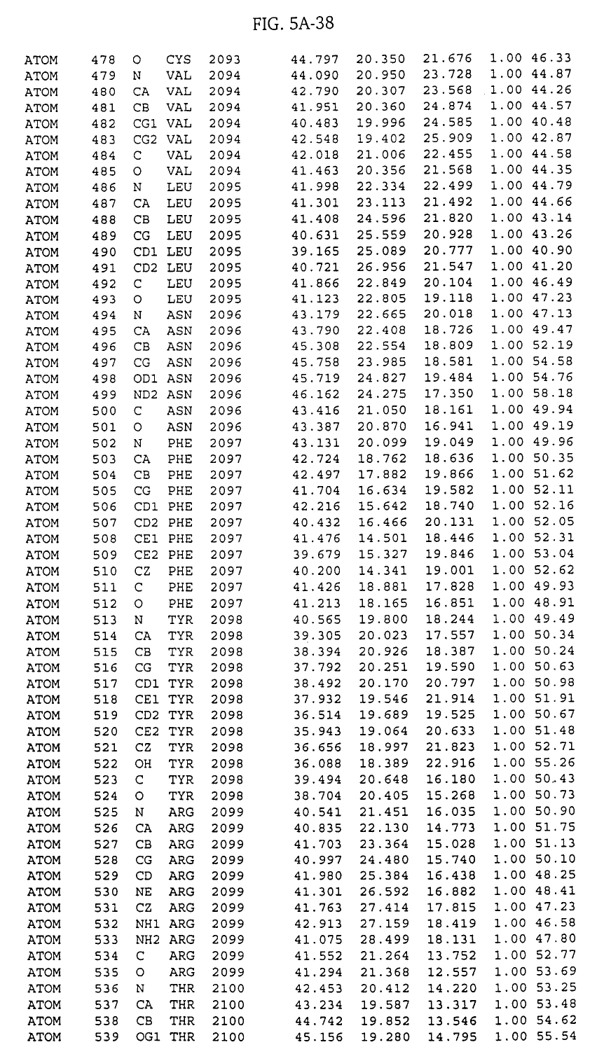

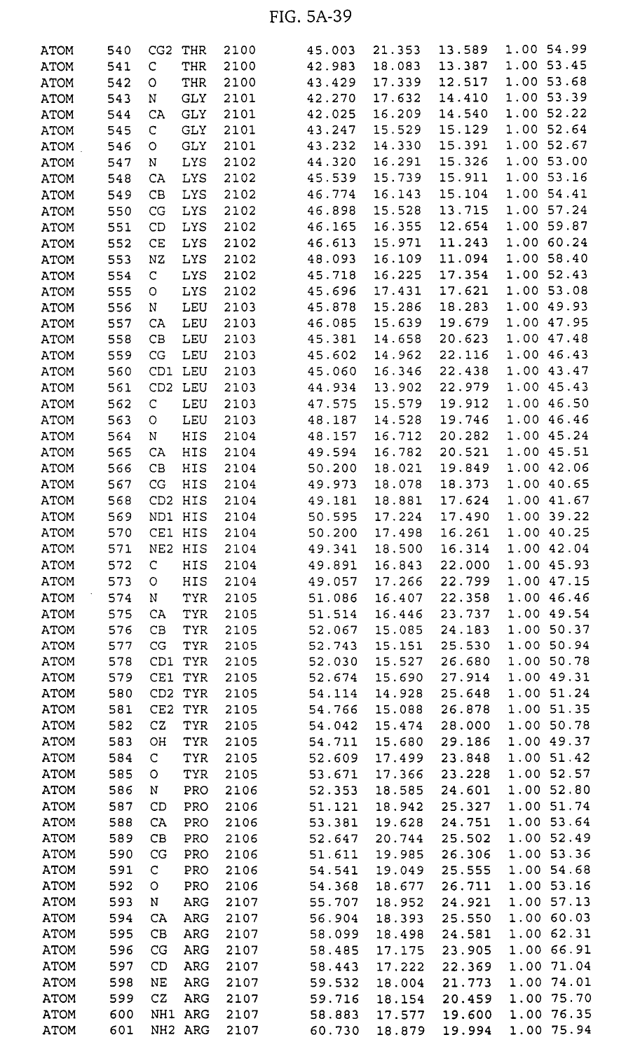

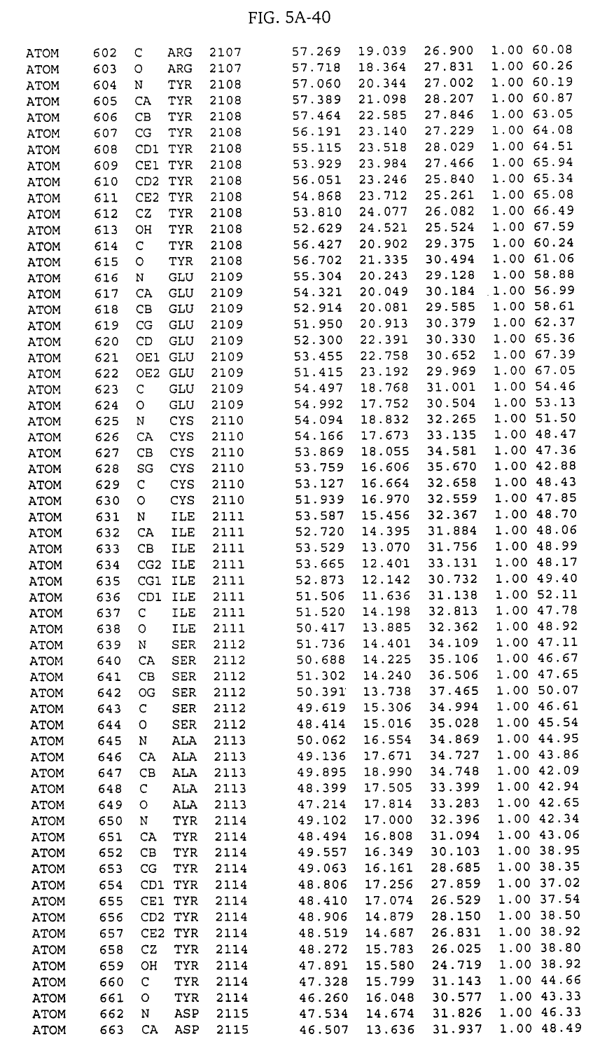

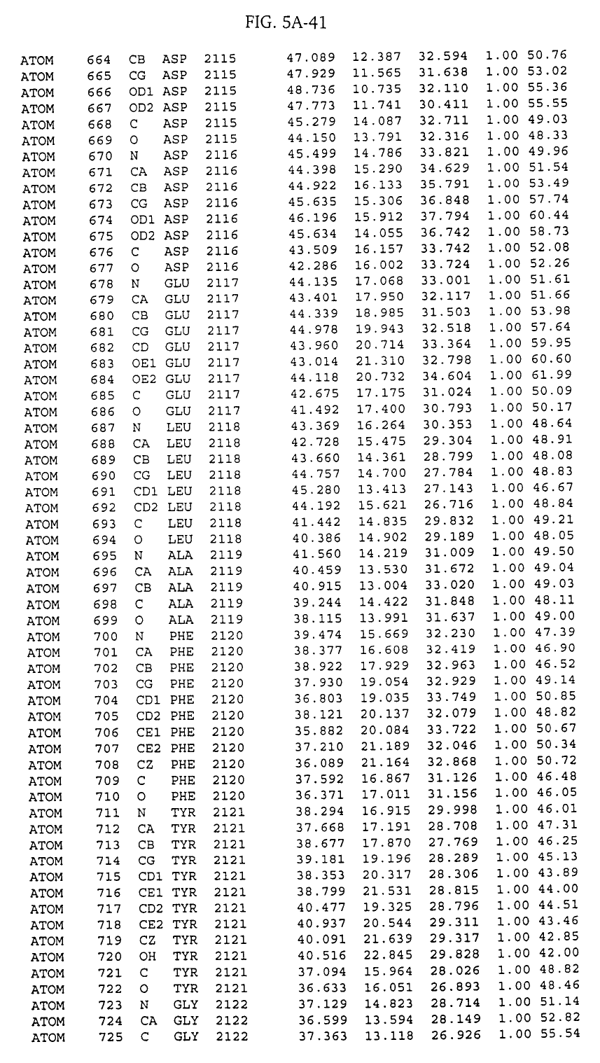

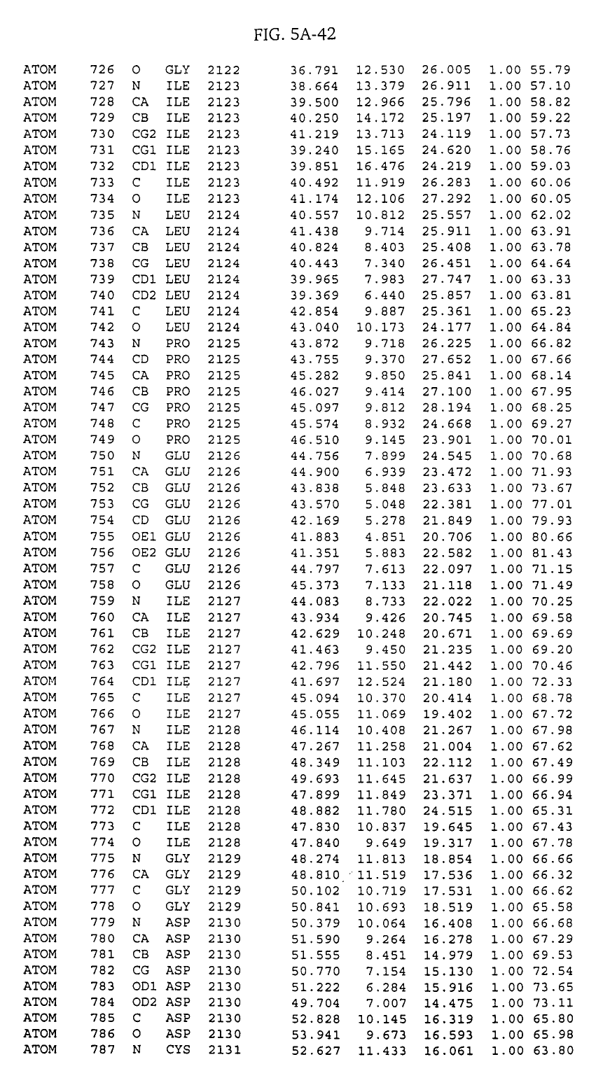

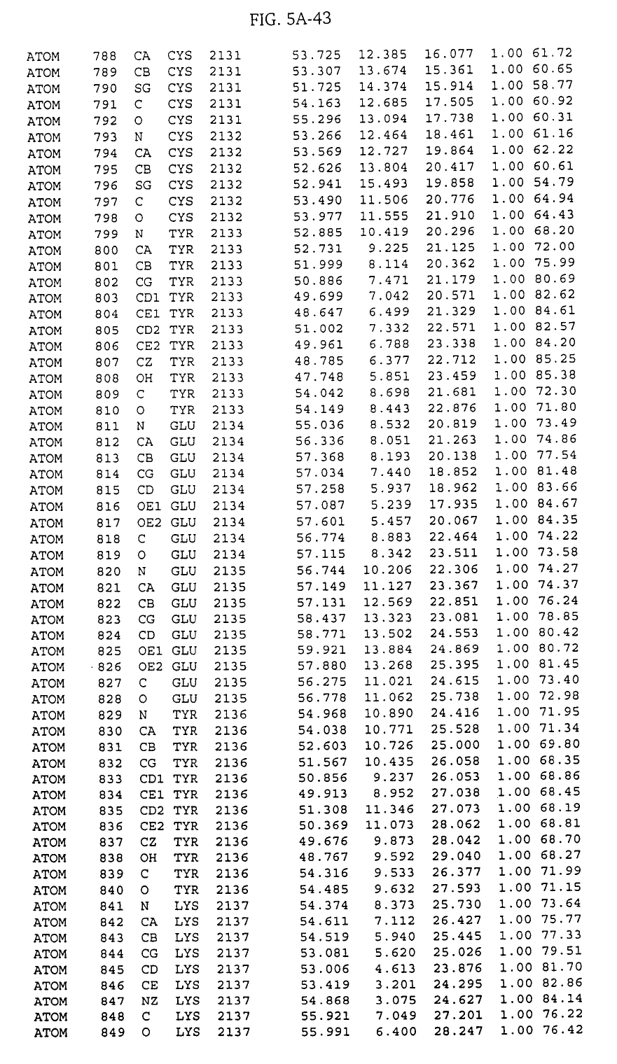

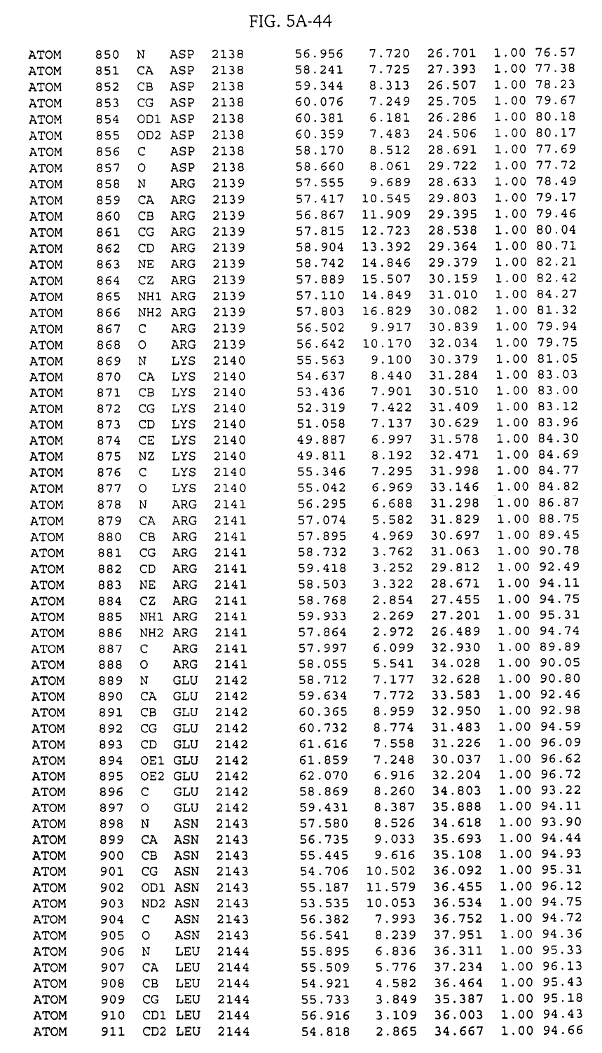

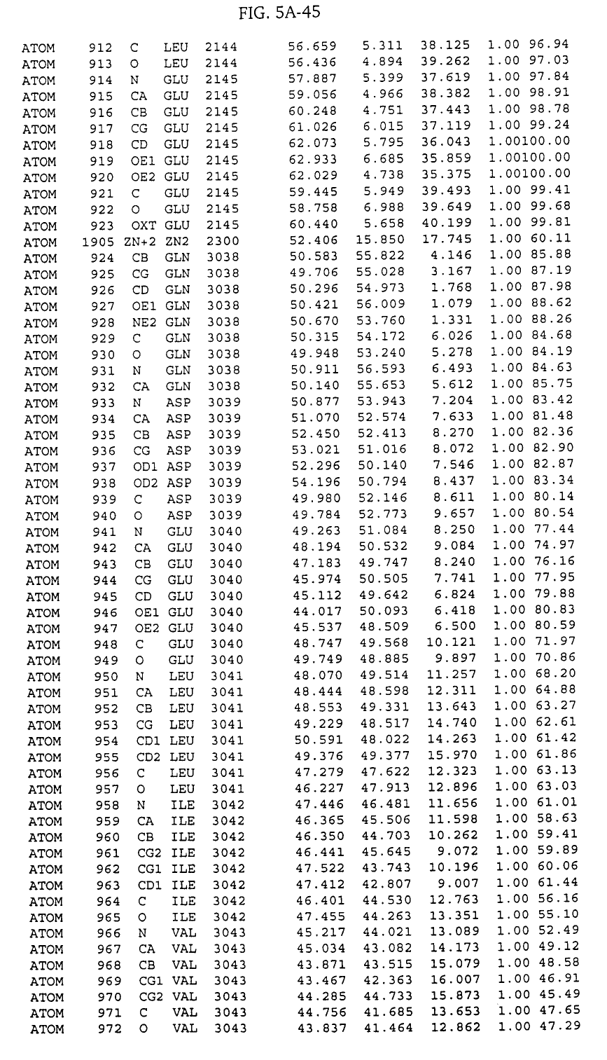

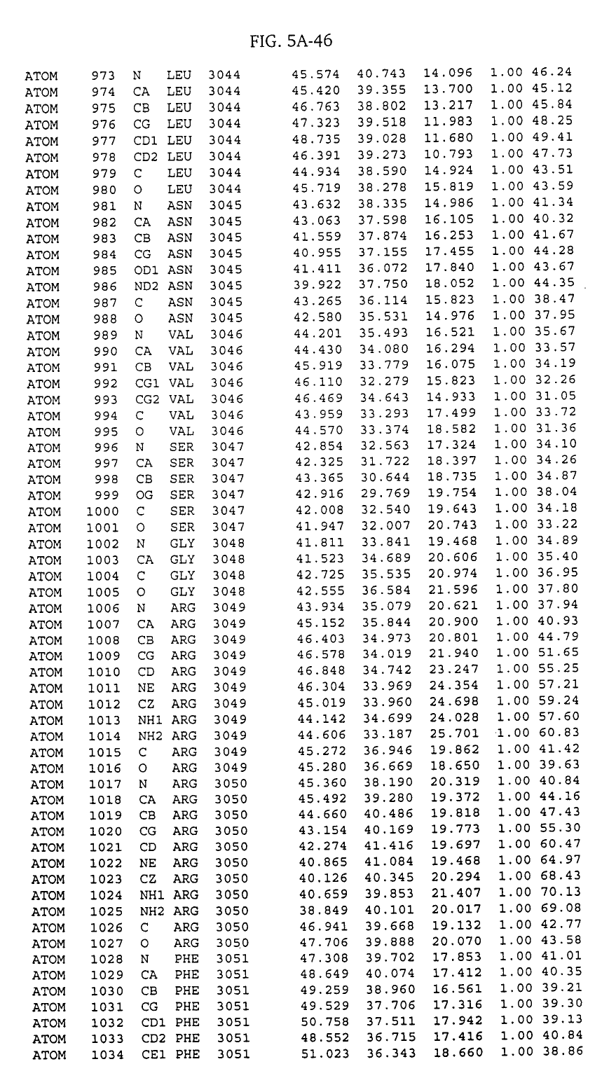

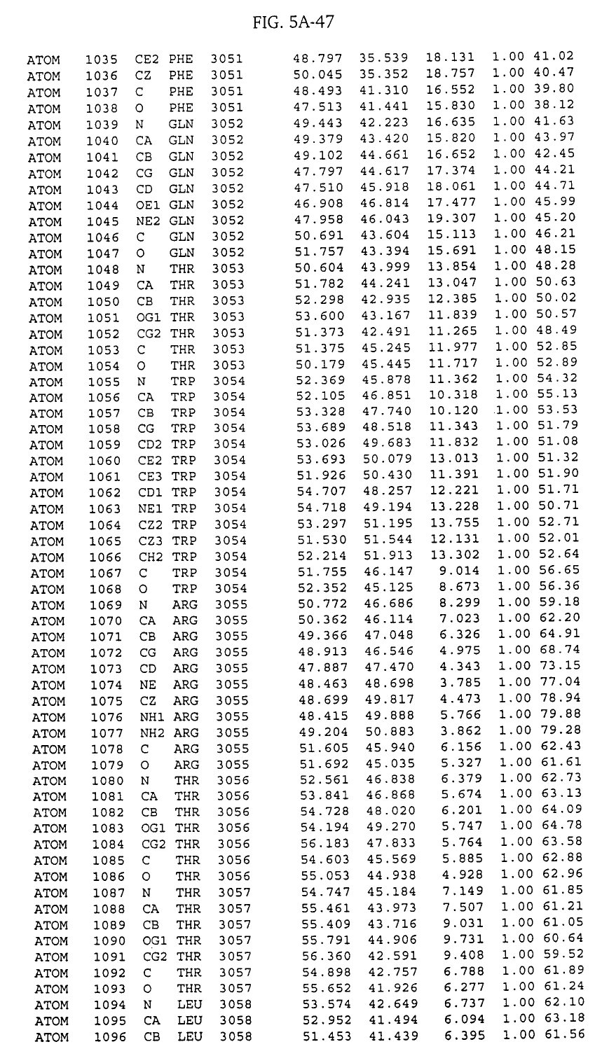

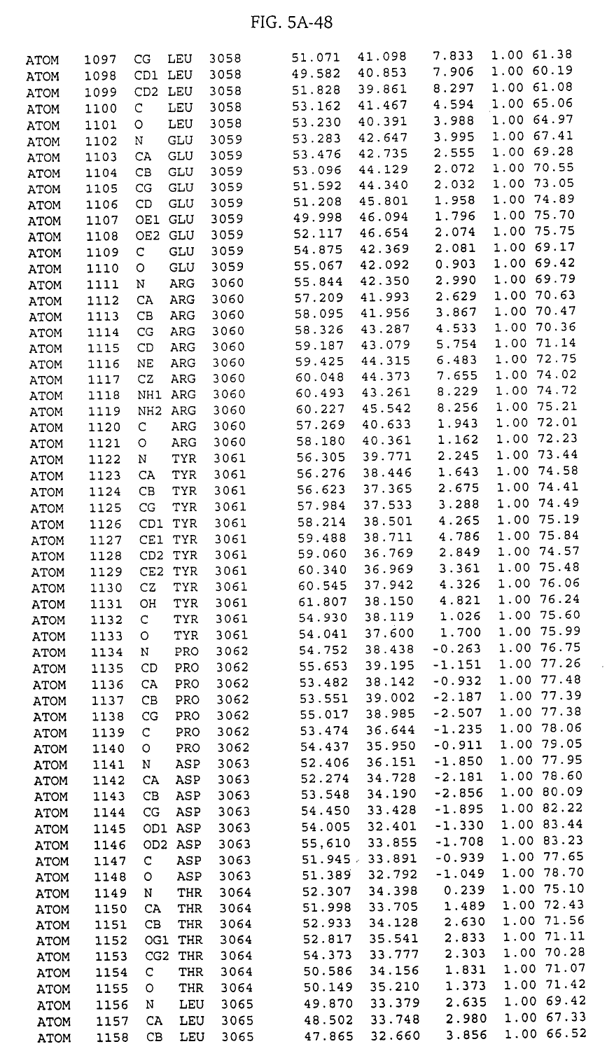

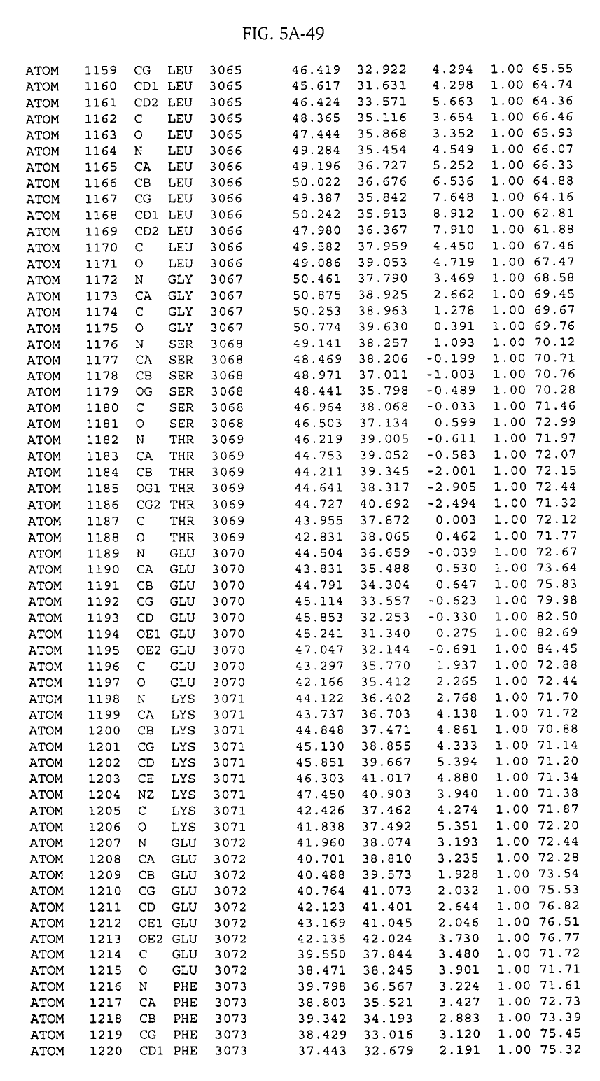

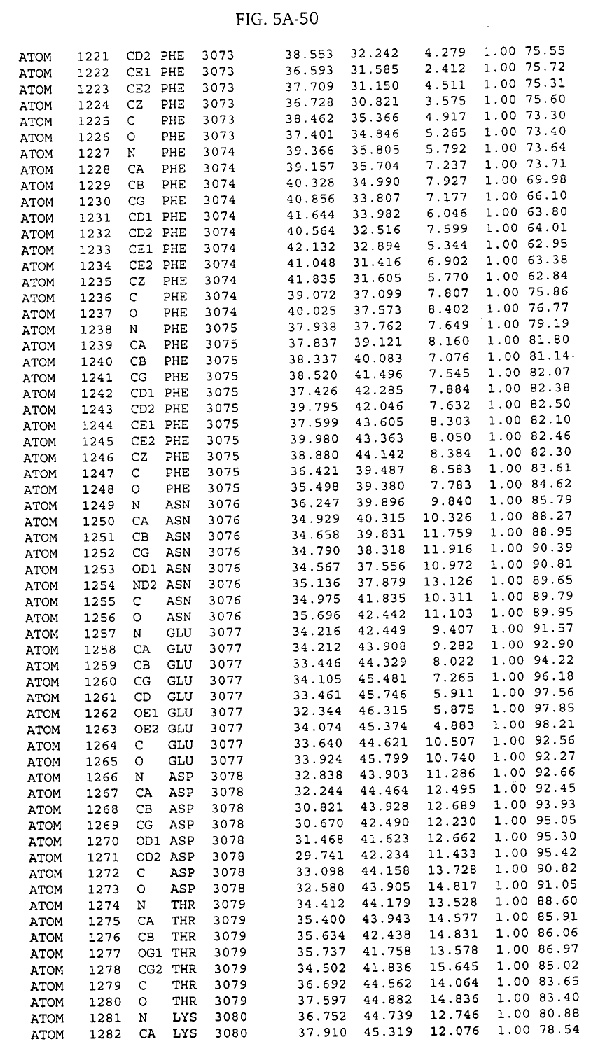

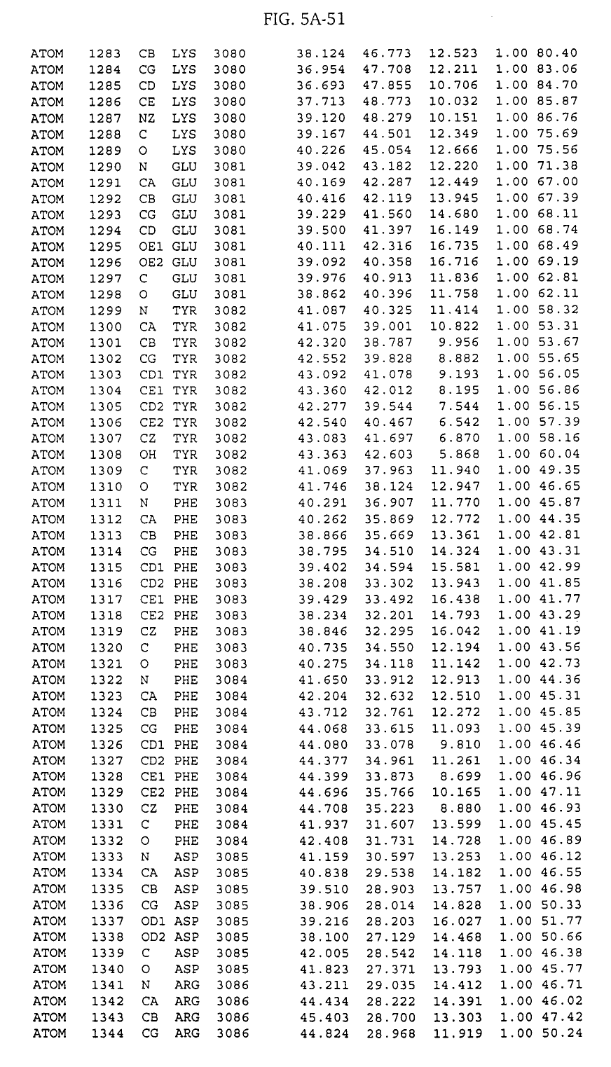

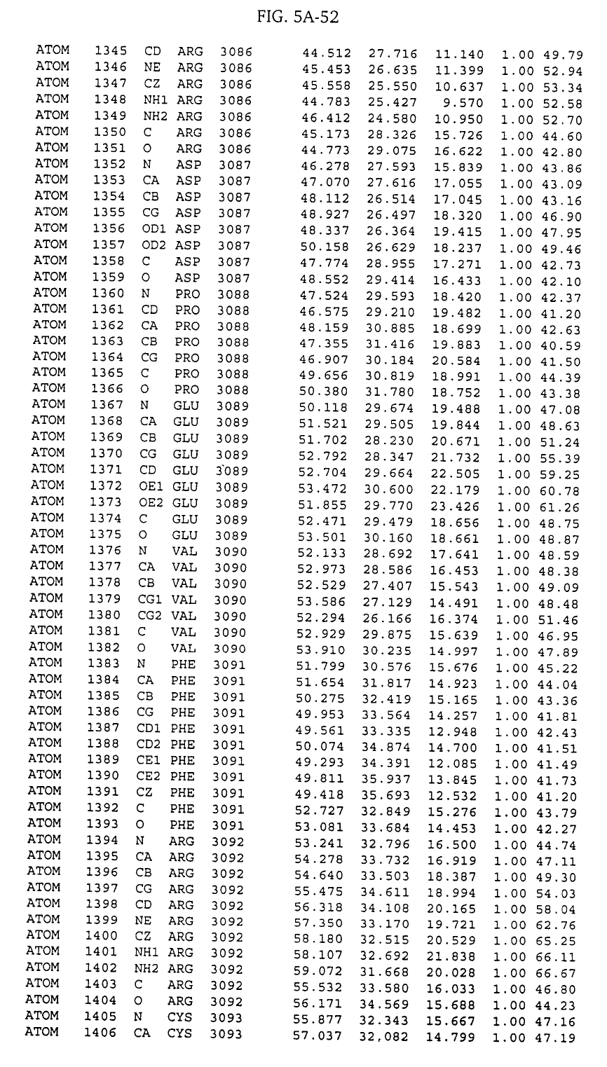

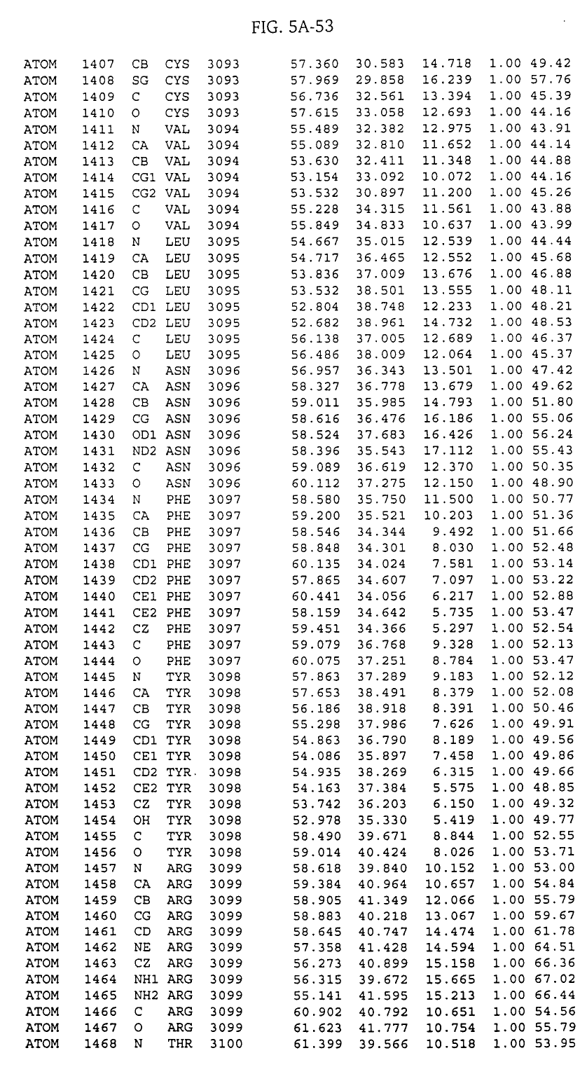

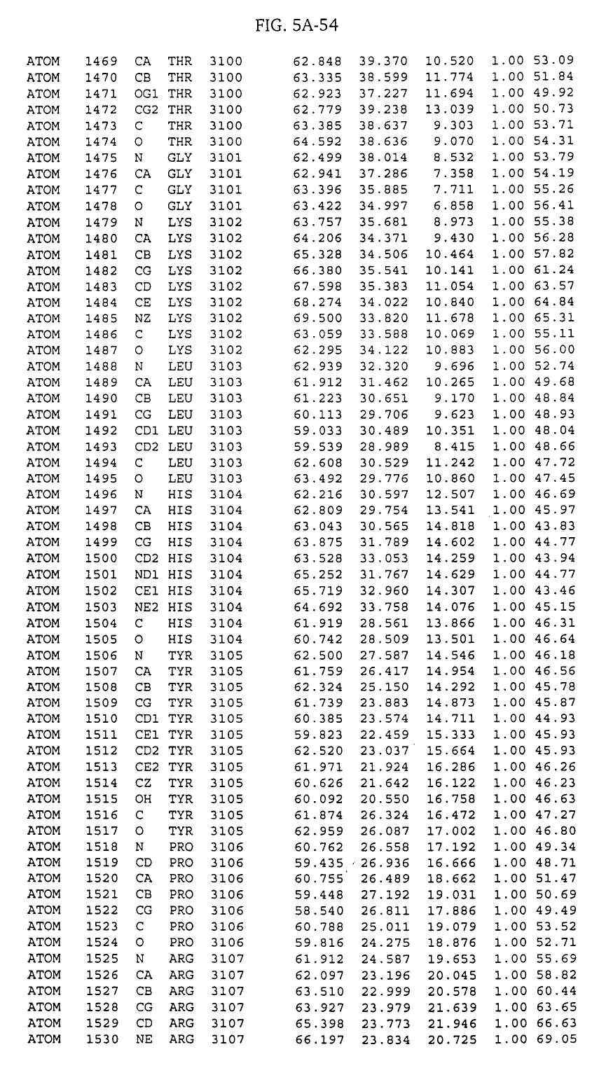

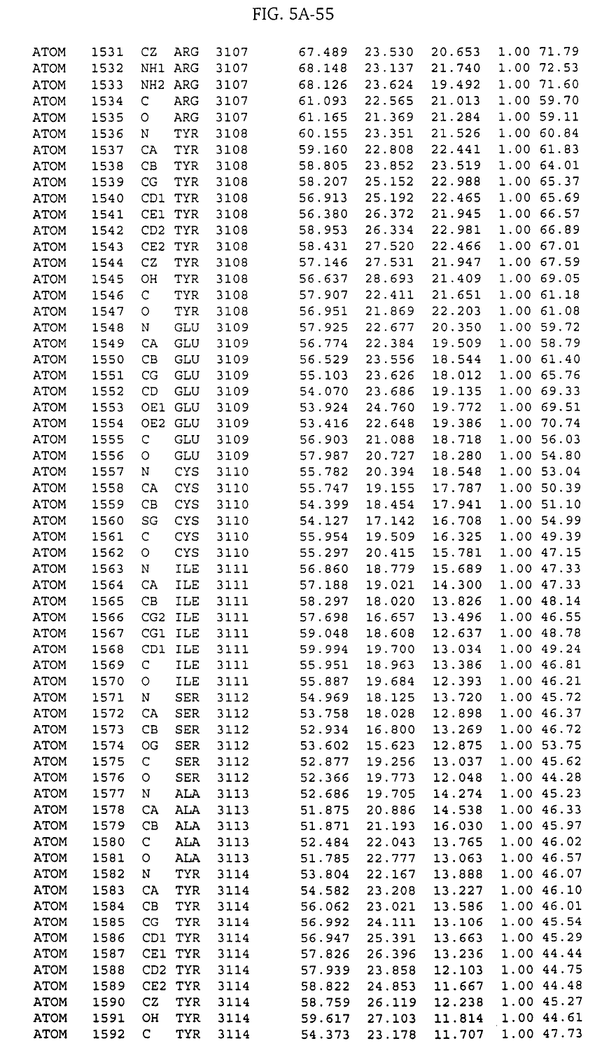

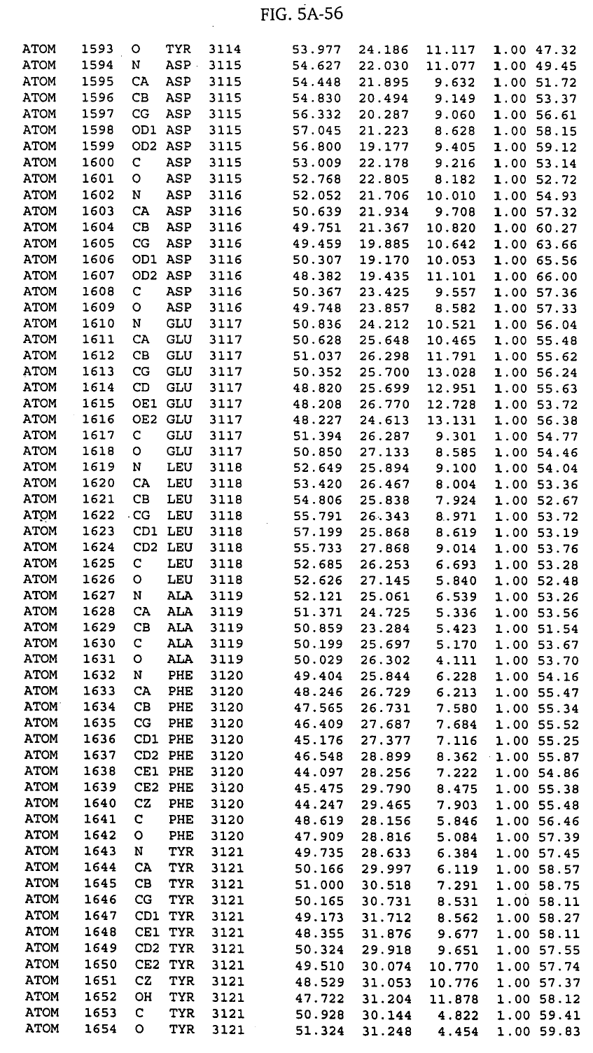

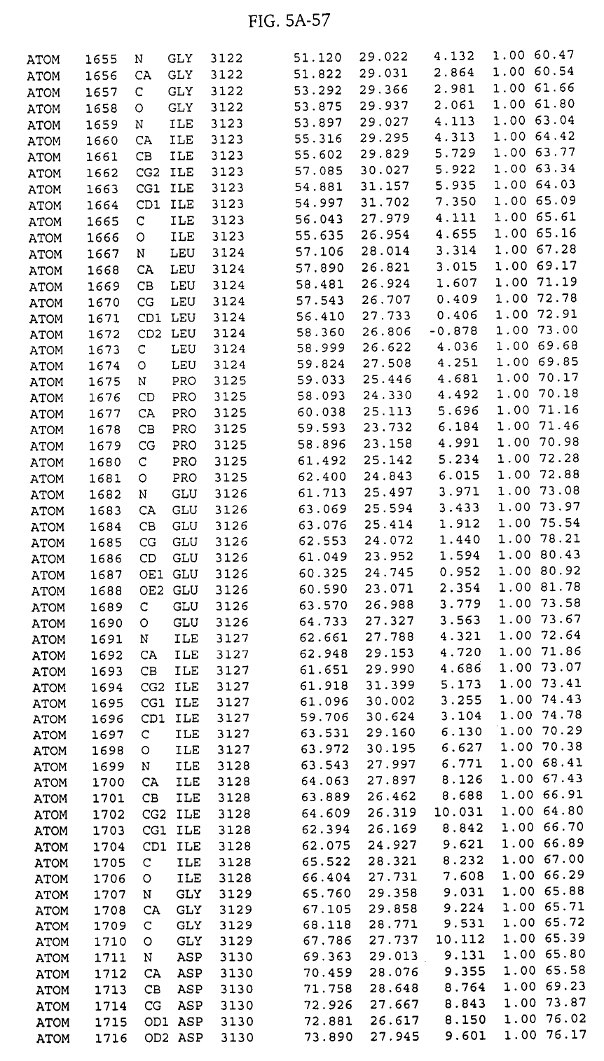

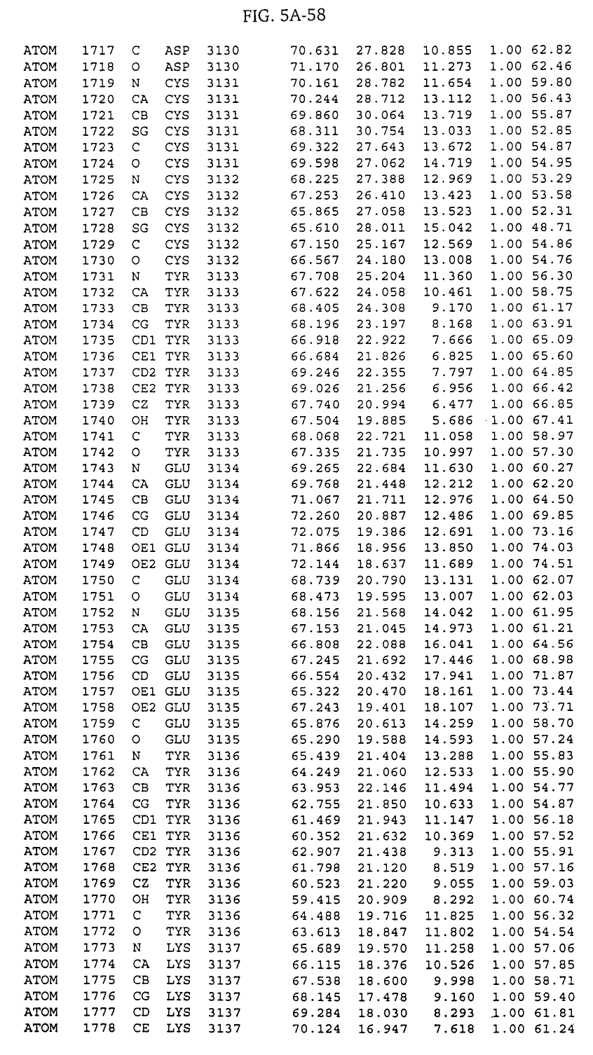

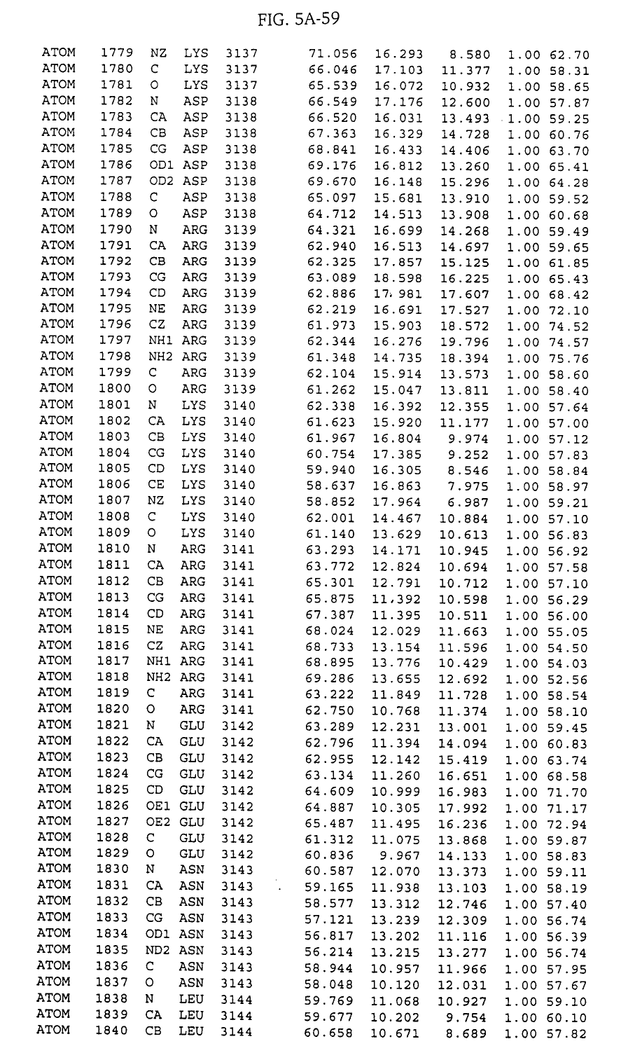

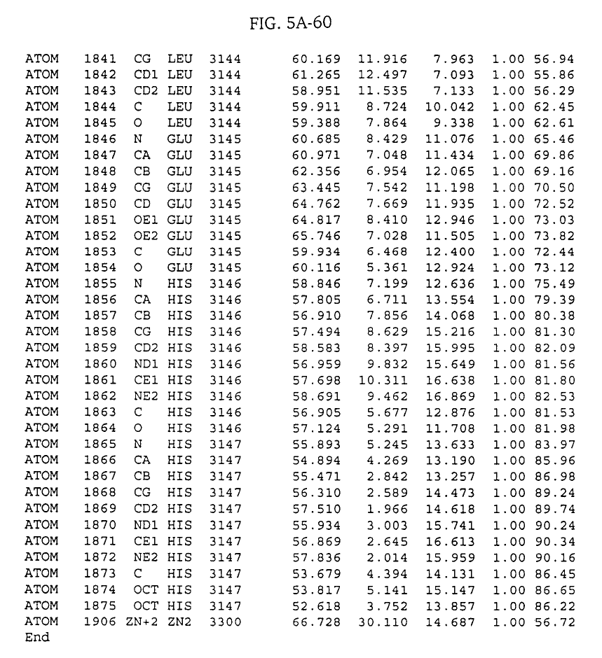

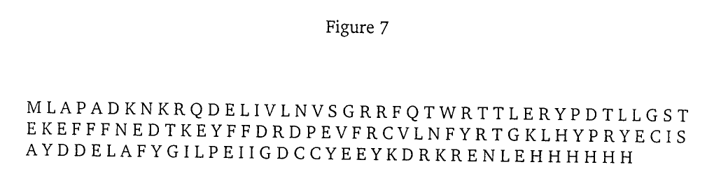

Unless otherwise noted, “Kv4.3 T1” is (i) the tetramerization domain (T1) (residues 29-143) of potassium voltage gated channel 3 (Kv4.3) as depicted within the sequence shown in FIG. 7, including conservative substitutions thereof; (ii) modeled Kv4.3 T1 as defined by residues 39-145, 1038-1145, 2039-2145 and/or 3038-3145 of FIG. 5, including conservative substitutions thereof, or (iii) a Kv4.3 T1 analogue having a portion of amino acid residues 39-145, 1038-1145, 2039-2145 and/or 3038-3145 of FIG. 5 that define at least one active site or putative active site of Kv4.3 T1, including conservative substitutions thereof. The complete amino acid sequence for Kv4.3 was deposited with the NCBI database under accession no. AAC05122.1, which is hereby incorporated by reference.

Unless otherwise indicated, “protein” or “molecule” shall include a protein, protein domain, polypeptide or peptide.

“Structural coordinates” are the Cartesian coordinates corresponding to an atom's spatial relationship to other atoms in a molecule or molecular complex. Structural coordinates may be obtained using x-ray crystallography techniques or NMR techniques, or may be derived using molecular replacement analysis or homology modeling. Various software programs allow for the graphical representation of a set of structural coordinates to obtain a three dimensional representation of a molecule or molecular complex. The structural coordinates of the present invention may be modified from the original sets provided in FIGS. 4 and 5 by mathematical manipulation, such as by inversion or integer additions or subtractions. As such, it is recognized that the structural coordinates of the present invention are relative, and are in no way specifically limited by the actual x, y, z coordinates of FIGS. 4 and 5.

An “agent” shall include a protein, polypeptide, peptide, nucleic acid (including DNA or RNA), molecule, compound or drug.

“Root mean square deviation” is the square root of the arithmetic mean of the squares of the deviations from the mean, and is a way of expressing deviation or variation from the structural coordinates described herein. The present invention includes all embodiments comprising conservative substitutions of the noted amino acid residues resulting in same structural coordinates within the stated root mean square deviation. It will be obvious to the skilled practitioner that the numbering of the amino acid residues of KCHIP-1 and Kv4.3 T1 may be different than that set forth herein, and may contain certain conservative amino acid substitutions that yield the same three dimensional structures as those defined by FIGS. 4 and 5 herein. Corresponding amino acids and conservative substitutions in other isoforms or analogues are easily identified by visual inspection of the relevant amino acid sequences or by using commercially available homology software programs (e.g., MODELLAR, MSI, San Diego, Calif.).

“Conservative substitutions” are those amino acid substitutions which are functionally equivalent to the substituted amino acid residue, either by way of having similar polarity, steric arrangement, or by belonging to the same class as the substituted residue (e.g., hydrophobic, acidic or basic), and includes substitutions having an inconsequential effect on the three dimensional structure of KCHIP-1 and Kv4.3 T1 with respect to the use of said structures for the identification and design of agents which interact with KCHIP-1 and Kv4.3 T1, as well as for molecular replacement analyses and/or for homology modeling.

An “active site” refers to a region of a molecule or molecular complex that, as a result of its shape and charge potential, favorably interacts or associates with another agent (including, without limitation, a protein, polypeptide, peptide, nucleic acid, including DNA or RNA, molecule, compound or drug) via various covalent and/or non-covalent binding forces. As such, an active site of the present invention may include, for example, the actual site of binding, as well as accessory binding sites adjacent or proximal to the actual site of binding that nonetheless may affect KCHIP-1 or Kv4.3 T1 activity upon interaction or association with a particular agent, either by direct interference with the actual site of substrate binding or by indirectly affecting the steric conformation or charge potential of the KCHIP-1 or Kv4.3 T1, thereby preventing or reducing binding to KCHIP-1 or Kv4.3 T1 at the actual site of binding. As used herein, an “active site” also includes analog residues of KCHIP-1 and Kv4.3 T1, which exhibit observable NMR perturbations in the presence of a binding ligand. While such residues exhibiting observable NMR perturbations may not necessarily be in direct contact with or immediately proximate to ligand binding residues, they may be critical KCHIP-1 or Kv4.3 T1 residues for rational drug design protocols.

The present invention first provides a crystallized Kv channel-interacting protein 1 (KCHIP-1) having one molecule of KCHIP-1 in the asymmetric unit. The crystal of the present invention effectively diffracts X-rays for the determination of the structural coordinates of KCHIP-1, and is characterized as having space group P4₁2₁2, unit cell parameters of a=b=50.34 Å, c=177.42 Å.

The present invention also provides a crystallized potassium channel Kv4.3 T1 domain (Kv4.3 T1) having two monomers of Kv4.3 T1 in the asymmetric unit. The crystal of the present invention effectively diffracts X-rays for the determination of the structural coordinates of Kv4.3 T1, and is characterized as having space group P4₁2₁2, unit cell parameters of a=b=84.23 Å, c=104.99 Å.

Using the crystals of the present invention, X-ray diffraction data can be collected by a variety of means in order to obtain the atomic coordinates of the molecules in the crystals. With the aid of specifically designed computer software, such crystallographic data can be used to generate a three dimensional structure. Various methods used to generate and refine a three dimensional structure of a molecular structure are well known to those skilled in the art, and include, without limitation, multiwavelength anomalous dispersion (MAD), multiple isomorphous replacement, reciprocal space solvent flattening, molecular replacement, and single isomorphous replacement with anomalous scattering (SIRAS).

Accordingly, the present invention also provides a three dimensional model of KCHIP-1 as derived by x-ray diffraction data of the KCHIP-1 crystal. The three dimensional model of KCHIP-1 is preferably defined by the relative structural coordinates for KCHIP-1 according to FIG. 4, ± a root mean square deviation from the backbone atoms of said amino acids of not more than 1.5 Å, preferably not more than 1.0 Å, and most preferably not more than 0.5 Å. More preferably, the three dimensional model of KCHIP-1 is defined by the relative structural coordinates of residues 12-192 of FIG. 4, including conservative substitutions thereof, or as a KCHIP-1 analogue having the relative structural coordinates of amino acid residues 12-192 of FIG. 4 that define at least one active site or putative active site of KCHIP-1, including conservative substitutions thereof. The three dimensional model of KCHIP-1 is useful for a number of applications, including, but not limited to, the visualization, identification and characterization of various active sites of KCHIP-1. The active site structures may then be used to design agents with interact with KCHIP-1.

The present invention also provides a three dimensional model of Kv4.3 T1 as derived by x-ray diffraction data of the Kv4.3 T1 crystal. The three dimensional model of Kv4.3 T1 is preferably defined by the structural coordinates shown in FIG. 5, ± a root mean square deviation from the backbone atoms of the amino acids of not more than 1.5 Å, preferably not more than 1.0 Å, and most preferably not more than 0.5 Å. More preferably, the three dimensional of Kv4.3T1 is defined by the relative structural coordinates of amino acid residues 39-145, 1038-1145, 2039-2145 and/or 3038-3145 of FIG. 5, including conservative substitutions thereof, or as a Kv4.3 T1 analogue having a portion of amino acid residues 39-145, 1038-1145, 2039-2145 and/or 3038-3145 of FIG. 5 that define at least one active site or putative active site of Kv4.3 T1, including conservative substitutions thereof.

The three dimensional model of Kv4.3 T1 is useful for a number of applications, including, but not limited to, the visualization, identification and characterization of various active sites of Kv4.3 T1. The active site structures may then be used to design agents with interact with Kv4.3 T1.

Another aspect of the present invention is directed to a method for identifying an agent that interacts with KCHIP-1, comprising the steps of: (a) generating a three dimensional model of KCHIP-1 using the relative structural coordinates of KCHIP-1 according to FIG. 4, ± a root mean square deviation from the backbone atoms of said amino acids of not more than 1.5 Å, preferably not more than 1.0 Å, and most preferably not more than 0.5 Å; and (b) employing said three-dimensional model to design or select an agent that interacts with KCHIP-1. More preferably, the three dimensional model of KCHIP-1 is defined by the relative structural coordinates of residues 12-192 of FIG. 4, including conservative substitutions thereof, or as a KCHIP-1 analogue having the relative structural coordinates of amino acid residues 12-192 of FIG. 4 that define at least one active site or putative active site of KCHIP-1, including conservative substitutions thereof.

In another embodiment, the present invention is directed to a method for identifying an agent that interacts with Kv4.3 T1, comprising the steps of: (a) generating a three dimensional model of Kv4.3 T1 using the relative structural coordinates of monomers 1 and/2 of Kv4.3 T1 according to FIG. 5, ± a root mean square deviation from the backbone atoms of said amino acids of not more than 1.5 Å, preferably not more than 1.0 Å, and most preferably not more than 0.5 Å; and (b) employing said three-dimensional model to design or select an agent that interacts with Kv4.3 T1. More preferably, the three dimensional of Kv4.3T1 is defined by the relative structural coordinates of amino acid residues 39-145, 1038-1145, 2039-2145 and/or 3038-3145 of FIG. 5, including conservative substitutions thereof, or as a Kv4.3 T1 analogue having a portion of amino acid residues 39-145, 1038-1145, 2039-2145 and/or 3038-3145 of FIG. 5 that define at least one active site or putative active site of Kv4.3 T1, including conservative substitutions thereof.

In the foregoing methods, the agent may be identified using computer fitting analyses utilizing various computer software programs that evaluate the “fit” between the putative active site and the identified agent, by (a) generating a three dimensional model of the putative active site of a molecule or molecular complex using homology modeling or the atomic structural coordinates of the active site, and (b) determining the degree of association between the putative active site and the identified agent. Three dimensional models of the putative active site may be generated using any one of a number of methods known in the art, and include, but are not limited to, homology modeling as well as computer analysis of raw data generated using crystallographic or spectroscopy data. Computer programs used to generate such three dimensional models and/or perform the necessary fitting analyses include, but are not limited to: GRID (Oxford University, Oxford, UK), MCSS (Molecular Simulations, San Diego, Calif.), AUTODOCK (Scripps Research Institute, La Jolla, Calif.), DOCK (University of California, San Francisco, Calif.), Flo99 (Thistlesoft, Morris Township, N.J.), Ludi (Molecular Simulations, San Diego, Calif.), QUANTA (Molecular Simulations, San Diego, Calif.), Insight (Molecular Simulations, San Diego, Calif.), SYBYL (TRIPOS, Inc., St. Louis. MO) and LEAPFROG (TRIPOS, Inc., St. Louis, Mo.). The structural coordinates also may be used to visualize the three-dimensional structure of KCHIP-1 or Kv4.3 T1 using MOLSCRIPT (Kraulis, P J, J. Appl. Crystallogr. 24: 946-950 (1991)) and RASTER3D (Bacon, D. J. and Anderson, W. F., J. Mol. Graph. 6: 219-220 (1998)), for example.

The effect of such an agent identified by computer fitting analyses on KCHIP-1 or Kv4.3 T1 activity may be further evaluated by contacting the identified agent with KCHIP-1 or Kv4.3 T1 and measuring the effect of the agent on KCHIP-1 or Kv4.3 T1 activity. Depending upon the action of the agent on the active site of KCHIP-1 or Kv4.3 T1, the agent may act either as an inhibitor or activator of KCHIP-1 or Kv4.3 T1 activity.

Various molecular analysis and rational drug design techniques are further disclosed in U.S. Pat. Nos. 5,834,228, 5,939,528 and 5,865,116, as well as in PCT Application No. PCT/US98/16879, published WO 99/09148, the contents of which are hereby incorporated by reference.

The present invention is also directed to the agents or inhibitors identified using the foregoing methods. Such agents or inhibitors may be a protein, polypeptide, peptide, nucleic acid, including DNA or RNA, molecule, compound, or drug. Small molecules or other agents which interact with KCHIP-1 or Kv4.3 T1 may be useful in the treatment of diseases or conditions associated with Kv channel proteins.

The present invention may be better understood by reference to the following non-limiting Example. The following Example is presented in order to more fully illustrate the preferred embodiments of the invention, and should in no way be construed as limiting the scope of the present invention.

### EXAMPLE 1

1. Methods and Methods

A. KCHIP1

Expression and Purification. Human KCHIP1 was cloned into the expression vector pET-21b(+) and expressed in E. coli strain BL21(DE3). Cells were lysed in lysis buffer containing 25 mM Tris-Cl, pH 8.0, 200 mM NaCl, 4 mM CaCl₂, 2 mM p-mercaptoethanol, 5% glycerol, with the supplement of protease inhibitor tablets. Cellular debris was removed by centrifugation. KCHIP1 in the soluble extract was batch purified by Ni-NTA affinity resin (Qiagen). The eluate was dialyzed overnight at 4° C. against buffer containing 25 mM Tris-Cl, pH 8.0, 2.5% glycerol, 10 mM CaCl₂, 1 mM EDTA, 4 mM β-mercaptoethanol, then applied onto a Poros PEI column (PerSeptive Biosystems, Framingham, Mass.) and developed with a NaCl gradient. The KCHIP1 containing fractions were buffer exchanged to 25 mM MES, pH 6.1, 25 mM NaCl, 10 mM CaCl₂, 10 mM DTT and further purified by a Poros HS column. A final gel filtration step on an TSK G3000 (TosoHaas, Montgomeryville, Pa.) column was applied prior to the crystallization step.

Crystallization. The KCHIP protein solution was buffered with 25 mM MES, pH 6.1, 0.25 M NaCl, 5 mM CaCl₂ and 5 mM DTT. The concentration of the protein used for crystallization was approximately 6.0 mg/ml. Crystallization conditions were found using the Hampton crystallization screen kits by the hanging drop vapor diffusion method (McPherson 1976). An optimized condition for growing KCHIP1 crystals was by mixing 3 μl of protein solution with 3 μl of precipitant solution (25% PEG₃₃₅₀, 0.2 M ammonium chloride, 5 mM calcium chloride and 15 mM DTT) and equilibrated against 1 mL precipitant solution at room temperature. The crystals began to appear after three days. After weeks, these crystals stopped growing. The average size of crystals is about 0.3×0.2×0.08 mm³.

Data collection and processing. The 30.0-2.3 Å resolution data were collected using Quantum 4 CCD area detector with the wavelength of 1.45 Å at ALS (Bekerley, Calif.). The oscillation angle for each image was 0.5 degree, and the X-ray exposure time was 20 seconds per image. The data were collected at −130° C. and were processed using DENZO and SCALEPACK (Otwinowski & Minor 1997). The R merge for full and partial reflections was 6.6%. The KCHIP crystal belongs to space group P4₁2₁2, with cell dimensions of a=b=50.03 Å, c=177.42 Å. The mosaicity of this crystal is 0.46°. There is one molecule per asymmetric unit. The statistics of data is shown in Table 1.

Structure determination and refinement. The KCHIP1 crystal structure has been determined by combining crystallographic modeling and molecular replacement method with the model of Neurocalcin (1BJF) structure. The sequence identity between the KCHIP and Neurocalcin is 43%.

The structural refinement was carried out by the program CNS (Brünger et al., 1998). The initial molecular model includes 120 amino acid residues without calcium ions. The model was refined against 15-2.3 Å X-ray data. The progress of the refinement was monitored with the geometry of the protein molecule and the electron density maps, and the values of the crystallographic R-factor. The initial R-factor was 52%. After rigid-body minimization, conjugating gradient minimization, heating stage, slow cooling stage in the range from 6000K to 300K, energy minimization, B-factor refinement and positional refinement, the R-factor went down to 32%. The difference maps contoured at five sigma level above the background show two calcium ions in the molecular structure. Some side-chains and a few main-chain loops were rebuilt using the interactive graphics system (QUANTA). The rebuilt model plus the calcium ions as the new model was refined. The R-factor was down to 27.6% and R_{free} of 30%. The final model contains 181 amino acids, two Ca²⁺ and 123 water molecules and corresponds to the R_{work}=0.224 and R_{free}=0.273 for the X-ray data from 15-2.3 Å. The statistics of the model refinement for KCHIP1 are summarized in Table 1. B. Kv4.3 T1 Domain

Expression and Purification. The T1 domain of Kv4.3 was expressed from a pET-21, a vector transformed in an Escherichia coli BL21-DE3 strain. The bacteria were grown in a fermentor at 37° C. in LB broth (10 liter) with Ampicillin (100 μg/ml) and supplemented with 0.5 mM ZnCl₂. The cells were induced with 1 mM IPTG at O.D. 0.5-0.8 and let grown for 4 hours. Excepted for the lysis realized on ice and the NTA-Ni²⁺ column (Qiagen) carried out at 4° C., all purification steps were carried out at room temperature.

After harvesting, the cells were resuspended in buffer A (20 mM Tris, pH 7.4, 100 mM NaCl, 1 mM 2-mercaptoethanol, 5 mM imidazole, 20 μM ZnCl₂) supplemented with 1 tablet per 50 ml of protease inhibitor (Complete™ EDTA-free, Roche). The lysis was realized with three passages through a microfluidizer. After centrifugation at 20,000 g for 30 minutes the cleared lysate was mixed for 1 hour to a Sepharose-NTA Ni²⁺ resin pre-equilibrated in buffer A. The resin was then poured in a column, then washed with 20 column volumes of buffer A followed by 20 column volumes of buffer A modified from 5 to 25 mM imidazole. The protein was eluted with buffer B (20 mM Tris, pH 7.4, 100 mM NaCl, 0.5 mM ZnCl₂, 10 mM 2-mercaptoethanol, 200 mM imidazole) and directly applied to a Poros HQ-50 column (Perseptive Biosystem). The HQ column was washed with buffer C (20 mM Tris, pH 7.4, 100 mM NaCl, 0.5 mM ZnCl₂, 10 mM 2-mercaptoethanol) and eluted with a salt gradient of 100-700 mM NaCl. T1-purest fractions were pooled, brought up to 3 mg/ml using the Millipore concentrators and sized on a G3000SW (Tosohaas) at 3 ml/min in buffer C. The protein eluting with a retention time matching a tetrameric conformation was collected and concentrated for crystallography studies.

Crystallization. Crystallization conditions for the T1 were determined from the sparse matrix screen (Emerald BioStructures). Screening was done using hanging drop vapor diffusion by combining 1 μl of protein solution (6˜7 mg/ml in 25 mM Tris, pH 7.4, 100 mM NaCl, 10 mM DTT and 1 mM ZnCl₂) with 1 μl of well solution (1 mL) at both 18° C. and 4° C. Small pyramid-like crystals appeared overnight at 4° C. in a mother liquor consisting of 2.0M NaH₂PO₄K₂HPO₄, 0.2M Li₂SO₄ and 0.1M CAPS, pH 10.5. The condition was further optimized by reducing the phosphate salt concentration to 1.4-1.5 M. The crystals grew to their maximum size (0.15×0.15×0.1 mm³) in about two weeks. The crystal belongs to space group P4₁2₁2 (a=b=84.23 Å, c=104.99 Å), there are two monomers per asymmetric unit and contains 60% solvent.

Data collection and processing. The crystals were transferred to the mother liquor, containing 30% glycerol, then flash cooled in liquid nitrogen. A single crystal was used for the data collection at beamline 5.0.2 at Advanced Light Source with wavelength 1.1 Å, using a Quantum 4 CCD detector at—130° C. All the data were integrated with DENZO and then scaled and merged with SCALEPACK (HKL 1.96.1). The statistics of data is summarized in Table 2.

Structure determination and refinement. T1 of Kv4.3 was located using the model of Shaw T1 (PDB code 3 kvt) dimer (constructed from the monomer based on its symmetry) in rotation and translation searches with AmoRe (Nevaza, 1994). All residues of Shaw T1 were used without truncation. The second largest peak provided by the rotation search generated a good translation function solution. The rigid-body refined model gave R factor of 49.1% and correlation coefficient of 36.4 for all data from 10-3.5 Å.

The search model was immediately subjected to simulated annealing refinement using CNS (Brünger et al. 1998). This resulted in R_{work}=37.7% and R_{free}=47.2% for 20-2.7 Å data, with 5% randomly selected reflections for R_{free} calculation. In parallel, the phases from the rigid-body refined model were calculated, and were used in density modification routing in CNS. The generated density modification map has good quality to identify the most of different residues between ShawT1 and Kv4.3 T1, and was used as the initial map for the modeling. The Kv4.3 T1 model was rebuilt with the correct sequence. After cycles of rebuilding, annealing or minimization and individual B factor refinements, the R factors converged to R_{work}=22.8% and R_{free}=27.3% for all data from 20-2.6 Å. The final model contains two monomers (residues 39-145 for monomer 1 and 1038-1145 for monomer 2), 29 water molecules and two Zn²⁺. All φ and ψ angles lie in the allowed regions of the Ramachandran plot. The statistics of the refinement results is included in Table 2.

2. Results

Structure Determination—KCHIP1. The Kv channel-interacting protein 1 (KCHIP-1, residues 1-216, with 2 extra residues and six His-tag at the C-terminal) was produced in E. coli and purified as described above. The crystals belong to space group P4₁2₁2 (unit cell dimensions a=b=50.34 Å, c=177.42 Å), with one molecule per crystallographic asymmetric unit. Diffraction data were collected from a single crystal at the Advanced Light Source. The structure was solved by molecular replacement method using the program AmoRe (CCP4, 1994), with the bovine Neurocalcin Delta 2 (PDB code 1BJF) as a search model, and refined to 2.3 Å resolution (R_{work}=22.4%, R_{free}=27.3%). The final model contains one molecule of the protein (residues 12-192), two calcium ions and 123 water molecules.

Structure Determination—Kv4.3 T1. The T1 domain (residues 29-143, with 1 and 2 extra residues at the N- and C-termini respectively, and six His-tag at the C-terminal) of potassium channel Kv4.3 was expressed in E. coli and purified as described above. The crystals belong to space group P4₁2₁2 (a=b=84.23 Å, c=104.99 Å) with two monomers per asymmetric unit and 60% solvent content. Diffraction data were collected at beamline 5.0.2 at the Advanced Light Source. The structure was determined by molecular replacement method using the Shaw T1 monomer (PDB code 3 kvt) as the search model. The structure was refined to 2.6 Å (R_{work}=22.8%, R_{free}=27.3%). The final model contains two monomers of the protein (residues 39-145 for monomer 1 and 1038-1145 for monomer 2) and 29 water molecules.

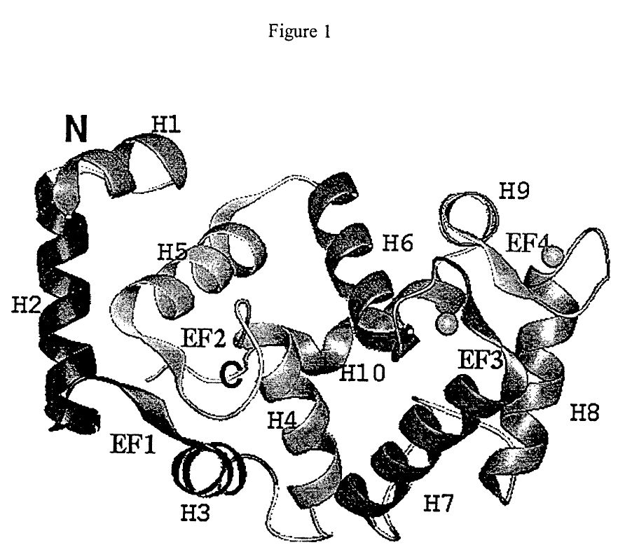

Overall Structure—KCHIP1. The structure of KCHIP1 molecule can be divided into two domains: residues 12-95 form the N-terminal domain and residues 96-192 constitute the C-terminal domain. Five α-helices (H1 through H5) are located in the N-terminal domain. The helices 2 and 3 form the EF1 hand and helices 4 and 5 form the EF2 hand. In the C-terminal domain, the EF3 and EF4 hands are formed by helices H6, H7 and H8, H9 respectively. H10 is the C-terminal helix. Connecting the ten α-helices are nine linker loops. The linker loops of EF1 and EF2, and EF3 and EF4 form two short antiparallel β-sheets, so that the four EF hands are grouped into two pairs (EF1-EF2 and EF3-EF4) (FIG. 1). Each EF-hand has a helix-loop-helix motif which is found in many other calcium binding proteins (Babu et al., 1988), such as Neurocalcin (Vijay-Kumar et al., 1999), Recoverin (Flaherty et al., 1993). The linker loops between each paired EF-hand are U shaped. The four EF-hands, similar to Neurocalcin and Recoverin, form a compact array on one face of the protein (FIG. 1).

The crystal structure of KCHIP revealed four potential Ca²⁺ binding EF-hands. However the X-ray data from calcium anomalous scattering diffraction as well as the difference electron density map showed only two Ca²⁺ bound to the KCHIP molecule. They are located at the EF3 and EF4 calcium binding sites. Comparing KCHIP and Neurocalcin, the EF3 and EF4 hands of both molecules are very similar and both of them involve Ca²⁺ binding. The calcium binding is not only functional character of the KCHIP but also structurally stabilizes the molecule. The conformations of EF1 and EF2 hands in Neurocalcin and KCHIP1 are different. The differences may be related to their different Ca²⁺ binding capabilities, as well as interactions with their target proteins. For example, in Neurocalcin, the EF2 hand binds a calcium ion, but in KCHIP1, it does not.

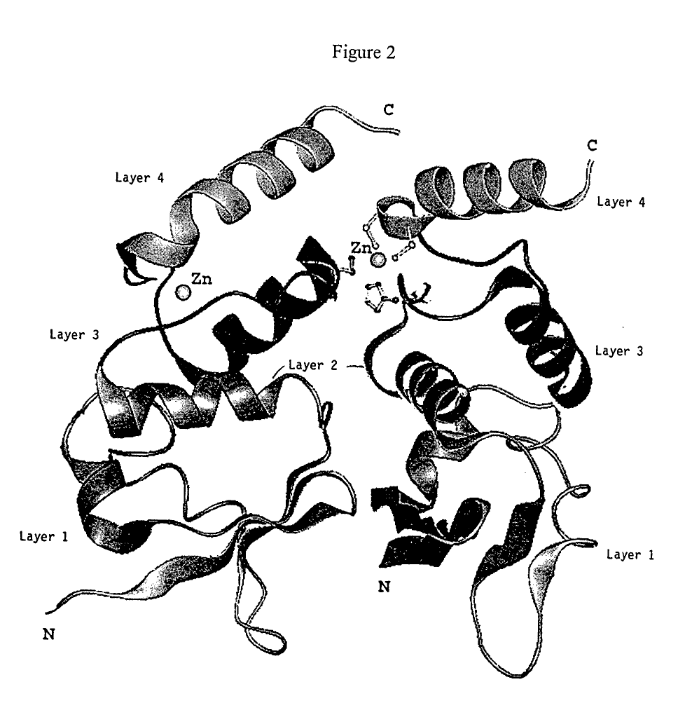

Overall Structure—Kv4.3 T1. The structure of the T1 monomer can be seen as consisting of two sub-domains or four layers (FIG. 2). These four layers are stacked along the four-fold axis of the homotetramer of T1. The N- and C-termini of each subunit are placed at opposite faces of the tetramer. The N-terminal layer 1 (38-85) is formed by two pairs of antiparallel β-strands interrupted by two short α-helices (one is distorted) between them. The two pairs of β-strands interact in parallel fashion to form a four-stranded β-sheet of layer 1. The following layer 2 consists of a single 15-residue long α-helix (86-101). A β-strand and an α-helix (102-123) form layer 3 of the structure. C-terminal layer 4 has a turn and a long 17-residue α-helix, which binds a Zn²⁺ with layer 3. There is one Zn²⁺ per monomer, which is tetrahedrally coordinated by Cys 131 and Cys 132 from layer 4, His 104 from layer 3, and a single cysteine (Cys 110) from layer 3 of the adjacent monomer (FIGS. 2 & 3). The HX₅CX₂₀CC Zn²⁺ binding motif is conserved in Shal, Shaw or Shab channels. An apparent function for the Zn²⁺ is to confer the conformation of layer 4 and promote inter-subunit contacts. The Zn²⁺ may also play an important role in differentiating Shad, Shal or Shaw like channels from Shaker like T1 in assembly, since the Zn²⁺ binding sequence motif and Zn²⁺ are not present in Shaker like channels.

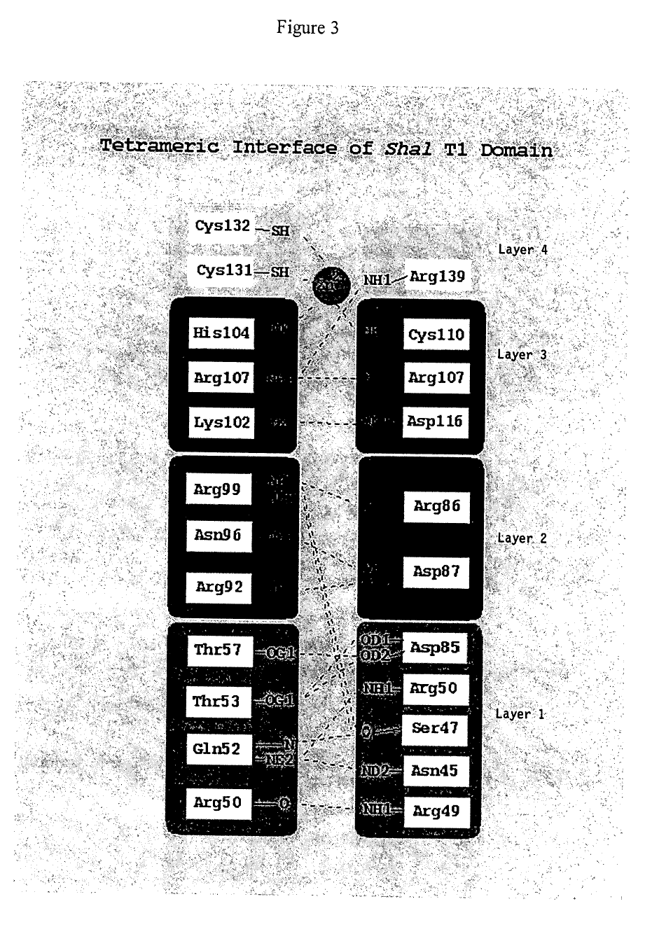

Tetramerization Interface of T1 Domain. There are 24 highly conserved residues in the T1 domain across all four types of K⁺-channels (Kv1, Kv2, Kv3 and Kv4) (Kreusch et al., 1998). Most of these conserved residues are buried within the T1 core, suggesting that overall fold of T1 is well preserved among potassium channel proteins. Within the T1 tetramer, each subunit buries ˜960 Å² of its solvent-accessible surface area at its two subunit interfaces. The majority interactions between subunits are polar (FIG. 3). In layer 1, there are 7 polar interactions, involving 8 residues. There are 6 interactions contributed from 6 residues in layer 2. Three residues in layer 3 form 4 interactions, including one to layer 4. Layer 4 mainly contributes to the Zn²⁺ coordination through its two Cys residues. Two Cys residues from layer 4, one His from layer 3 with a Cys from second subunit form tetrahedral Zn²⁺ coordination site in the two subunit interfaces. Although the common T1 core exists in all four different K⁺-channels, each has its own association specificity. This specificity must be encoded in its tetramerization interface. Nineteen residues involved in the interface interactions are conserved in Kv4 subfamily, whereas only four of them are conserved across all four different K⁺-channels.

TABLE 1
Crystallographic data for KCHIP-1

| | |
|---|---|
| Structure | KCHIP-1 |

Data Collection

| | |
|---|---|
| Wavelength (Å) | 1.45 |
| Cell (a,b,c) (Å) | 53.13, 53.13, 176.96 |
| Space Group | P4₁2₁2 |
| Resolution range (Å) | 30-2.3 |
| Completeness (%) | 99.9 |
| Total observations | 59609 |
| Unique reflections | 10144 |
| Average I/s (I) | 25.64 (7.2) |
| ¹R_{sym} (%) | 6.6 (18.2) |
| Model Refinement | |
| Resolution range (Å) | 15-2.3 |
| ²R_{work} . (%) | 22.4 |
| R_{free} (%) | 27.3 |
| R.m.s. deviations | |
| Bonds (Å) | 0.006 |
| Angles (°) | 1.117 |
| B-factors for bonded main-chain atoms (Å²) | 1.138 |

TABLE 2
Crystallographic data for T1

| Structure | Shal T1 |
|---|---|
| Data Collection | |
| Wavelength (Å) | 1.1 |
| Cell (a,b,c) | 84.23, 84.23, 104.99 |
| Space group | P4₁2₁2 |
| Resolution range (Å) | 20-2.6 |
| Completeness (%) | 99.6 |
| Total observations | 108494 |
| Unique reflections | 12263 |
| Average I/s (I) | 18.5 (2.5) |
| ¹R_{sym} (%) | 7.1 (71.6) |
| Model Refinement | |
| Resolution range (Å) | 20-2.6 |
| ²R_{work} (%) | 22.8 |
| R_{free} (%) | 27.3 |
| R.m.s. deviations | |
| bonds (Å) | 0.007 |
| angles (°) | 1.15 |
| B-factors for bonded main-chain atoms (Å²) | 1.88 |

¹R_{sym} = Σ | Iₙ − <Iₙ> | / ΣIₙ, where <Iₙ> is the average intensity over symmetry equivalents. Number in parentheses reflect statistics for the last shell;
²R_{work} = Σ | |F_{obs}| − |f_{calc}| / Σ |F_{obs}|, R_{free} is equivalent to R_{work}, but calculated for a randomly chosen 5% of reflections omitted from the refinement process.

### REFERENCES

An, et al., (2000) Modulation of A-type potassium channels by a family of calcium sensors. Nature, 403: 553-556.

Bähring, R., Dannenberg, J., Peters, H. C., Leicher, T., Pongs, O. and Isbrandt, D. (2001) Conserved Kv4 N-terminal domain critical for effects of Kv channel-interacting protein 2.2 on channel expression and gating. J. Biol. Chem., 276, 23888-23894.

Babu, Y. S. Bugg, C. E. and Cook, W. J. (1988) Structure of calmodulin refined at 2.2 Å resolution. J. Mol. Biol., 204, 191-204,.

Bixby, K. A., Nanao, M. H., Shen, N. V., Kreusch, A., Bellamy, H., Pfaffinger, P. J. and Choe, S. (1999) Zn²⁺-binding and molecular determinants of tetramerization in voltage-gated K⁺ channels. Nature Structural Biology, 6, 38-43.

Brünger, A. T. et al. (1998) Crystallography and NMR system: a new software suite for macromolecular structure determination. Acta Crystallogr., D54, 905-921.

CCP4 (1994) The CCP4 suite: programs for X-ray crystallography. Acta Crystallogr., D50, 760-763.

Dixon J E., Shi W., Wang H S., McDonald C., Yu H., Wymore R S., Cohen IS., and McKinnon D (1996) Role of the Kv4.3 K⁺ channel in ventricular muscle. A molecular correlate for the transient outward current. Circ. Res., 79, 659-668.

Flaherty, K. M., Zozulya, S., Stryer, L. and McKay, D. B. (1993) Three-dimensional structure of recoverin, a calcium sensor in vision. Cell, 75, 709-716.

Frank An, W. et al. (2000) Modulation of A-type potassium channels by a family of calcium sensors. Nature, 403, 553-556.

Gulbis, J. M., Zhou, Ming, Mann, S. and Mackinnon, R. (2000) Structure of the cytoplasmic β subunit-T1 assembly of voltage-dependent K⁺ channels. Science, 289, 123-127.

Hille B. (1992) Ionic Channels of Excitable Membranes, Second Edition, Sunderland, Mass.

Hoffman D. A., Magee J. C., Colbert C. M., and Johnston D. (1997) K⁺ channel regulation of signal propagation in dendrites of hippocampal pyramidal neurons. Nature, 387, 869-875.

Jan, L. Y. and Jan, Y. N. (1990) How might the diversity of potassium channels be generated? Trends Neurosi., 13, 415-419.

Kreusch, A., Pfaffinger, P. J., Stevens, C. F. and Choe, S. (1998) Crystal structure of the tetramerization domain of the shaker potassium channel. Nature, 392, 945-948.

Li, M., Jan, J. M., and JAN, L. Y. (1992) Specification of subunit assembly by the hydrophilic amino-terminal domain of the Shaker potassium channel. Science, 257, 1225-1230.

McPherson, A. (1976). Methods Biochem. Anal., 23, 249-345.

Nevaza, Z. (1994) AmoRE-an automated package for molecular replacement. Acta Crstallogr., A50, 157-163.

Otwinowski, Z and Minor, W. (1997) Processing of X-ray diffraction data collected in oscillation mode. Methods in Enzymology, 276, 307-326.

Pongs, O., Leicher, T., Berger, M., Roeper, J., Bähring, R., Wray, D., Giese, K. P., Silva, A. J. and Storm, J. F. (1999) Functional and molecular aspects of voltage-gated K⁺ channel beta subunits. Annals of the New York Academy of Sciences, 868, 344-55.

Serodio, P. & Rudy, B. (1998) Differential expression of Kv4 K⁺ channel subunits mediating subthreshold transient K⁺ (A-type) currents in rat brain. J. Neurophys., 79, 1081-1091.

Serodio, P., Vega-Saenz de Miera, E. & Rudy, B. (1996) Cloning of a novel component of A-type K+ channels operating at subthreshold potentials with unique expression in heart and brain. J. Neurophys., 75, 2174-2179.

Shen, N. V., Chen, X., Boyer, M. M., and Pfaffinger, P. (1993) Deletion analysis of K⁺ channel assembly. Neuron, 11, 67-76.

Sheng, M., Tsaur, M. L., Jan Y. N. & Jan, L. Y. (1992) Subcellular segregation of two A-type K⁺ channel proteins in rat central neurons. Neuron, 9, 271-284.

Vijay-Kumar, S. and Kumar, V. D. (1999) Crystal structure of recombinant bovine neurocalcin. Nature Str. Bio., 6, 80-88.

All publications mentioned herein above, whether to issued patents, pending applications, published articles, or otherwise, are hereby incorporated by reference in their entirety. While the foregoing invention has been described in some detail for purposes of clarity and understanding, it will be appreciated by one skilled in the art from a reading of the disclosure that various changes in form and detail can be made without departing from the true scope of the invention in the appended claims.

What is claimed is:

1. A crystallized Kv channel-interacting protein 1 (KCHIP-1) having one molecule of KCHIP-1 in the asymmetric unit.

2. The crystallized KCHIP-1 of claim 1, characterized as having space group P4₁2₁2, unit cell parameters of a=b=50.34 Å, c=177.42 Å.

3. A crystallized potassium channel Kv4.3 T1 domain (Kv4.3 T1) having two monomers of Kv4.3 T1 in the asymmetric unit.

4. The crystallized Kv4.3 T1 of claim 3, characterized as having space group P4₁2₁2, unit cell parameters of a=b=84.23 Å, c=104.99 Å.

5. A three dimensional model of KCHIP-1 defined by the relative structural coordinates for KCHIP-1 according to FIG. 4, ± a root mean square deviation from the backbone atoms of said amino acids of not more than 1.5 Å.

6. The three dimensional model of claim 5, wherein the +a root mean square deviation from the backbone atoms of said amino acids is not more than 1.0 Å.

7. The three dimensional model of claim 5, wherein the +a root mean square deviation from the backbone atoms of said amino acids is not more than 0.5 Å.

8. A three dimensional model of Kv4.3 T1 defined by the relative structural coordinates for Kv4.3 T1 according to FIG. 5, ± a root mean square deviation from the backbone atoms of said amino acids of not more than 1.5 Å.

9. The three dimensional model of claim 8, wherein the +a root mean square deviation from the backbone atoms of said amino acids is not more than 1.0 Å.

10. The three dimensional model of claim 8, wherein the ± a root mean square deviation from the backbone atoms of said amino acids is not more than 0.5 Å.

11. A method for identifying an agent that interacts with KCHIP-1, comprising the steps of:
(a) generating a three dimensional model of KCHIP-1 using the relative structural coordinates of KCHIP-1 according to FIG. 4, ± a root mean square deviation from the backbone atoms of said amino acids of not more than 1.5 Å; and
(b) employing said three-dimensional model to design or select an agent that interacts with KCHIP-1.

12. The method of claim 11, wherein the ± a root mean square deviation from the backbone atoms of said amino acids is not more than 1.0 Å.

13. The method of claim 11, wherein the ± a root mean square deviation from the backbone atoms of said amino acids is not more than 0.5 Å.

14. The method of claim 11, further comprising the steps of: (c) obtaining the identified agent; and (d) contacting the identified agent with KCHIP-1 in order to determine the effect the agent has on KCHIP-1 activity.

15. A method for identifying an agent that interacts with Kv4.3 T1, comprising the steps of:
(a) generating a three dimensional model of Kv4.3 T1 using the relative structural coordinates of Kv4.3 T1 according to FIG. 5, ± a root mean square deviation from the backbone atoms of said amino acids of not more than 1.5 Å; and
(b) employing said three-dimensional model to design or select an agent that interacts with Kv4.3 T1.

16. The method of claim 15, wherein the ± a root mean square deviation from the backbone atoms of said amino acids is not more than 1.0 Å.

17. The method of claim 15, wherein the +a root mean square deviation from the backbone atoms of said amino acids is not more than 0.5 Å.

18. The method of claim 15, further comprising the steps of: (c) obtaining the identified agent; and (d) contacting the identified agent with Kv4.3 T1 in order to determine the effect the agent has on Kv4.3 T1 activity.

19. An agent identified by the method of claim 11.

20. An agent identified by the method of claim 15.


SEQUENCE LISTING (3 sequences)

SEQ ID NO: 1 - protein, 223 aa, Homo sapiens
MGAVMGTFSSLQTKQRRPSKDKIEDDLEMTMVCHRPEGLEQLEAQTNFTKRELQVLYRGFKNECPSGVVNEETFKQIYAQFFPHGDASTYAHYLFNAFDTTQTGSVKFEDFVTALSILLRGTVHEKLRWTFNLYDINKDGYINKEEMMDIVKAIYDMMGKYTYPVLKEDTPRQHVDFFQKMDKNKDGIVTLDEFLESCQEDDNIMRSLQLFQNVMVEHHHHHH

SEQ ID NO: 2 - protein, 124 aa, Homo sapiens
MLAPADKNKRQDELIVLNVSGRRFQTWRTTLERYPDTLLGSTEKEFFFNEDTKEYFFDRDPEVFRCVLNFYRTGKLHYPRYECISAYDDELAFYGILPEIIGDCCYEEYKDRKRENLEHHHHHH

SEQ ID NO: 3 - protein, 29 aa, Artificial Sequence
  conserved binding motif
HXXXXXCXXXXXXXXXXXXXXXXXXXXCC